SEQUENCE LISTING (29 sequences)

SEQ ID NO: 1 - protein, 581 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 2 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 3 - protein, 379 aa, Homo sapiens
MAWRGWAQRGWGCGQAWGASVGGRSCEELTAVLTPPQLLGRRFNFFIQQKCGFRKAPRKVEPRRSDPGTSGEAYKRSALIPPVEETVFYPSPYPIRSLIKPLFFTVGFTGCAFGSAAIWQYESLKSRVQSYFDGIKADWLDSIRPQKEGDFRKEINKWWNNLSDGQRTVTGIIAANVLVFCLWRVPSLQRTMIRYFTSNPASKVLCSPMLLSTFSHFSLFHMAANMYVLWSFSSSIVNILGQEQFMAVYLSAGVISNFVSYVGKVATGRYGPSLGASGAIMTVLAAVCTKIPEGRLAIIFLPMFTFTAGNALKAIIAMDTAGMILGWKFFDHAAHLGGALFGIWYVTYGHELIWKNREPLVKIWHEIRTNGPKKGGGSK

SEQ ID NO: 4 - DNA, 1140 bp, Homo sapiens
atggcgtggcgaggctgggcgcagagaggctggggctgcggccaggcgtggggtgcgtcggtgggcggccgcagctgcgaggagctcactgcggtcctaaccccgccgcagctcctcggacgcaggtttaacttctttattcaacaaaaatgcggattcagaaaagcacccaggaaggttgaacctcgaagatcagacccagggacaagtggtgaagcatacaagagaagtgctttgattcctcctgtggaagaaacagtcttttatccttctccctatcctataaggagtctcataaaacctttattttttactgttgggtttacaggctgtgcatttggatcagctgctatttggcaatatgaatcactgaaatccagggtccagagttattttgatggtataaaagctgattggttggatagcataagaccacaaaaagaaggagacttcagaaaggagattaacaagtggtggaataacctaagtgatggccagcggactgtgacaggtattatagctgcaaatgtccttgtattctgtttatggagagtaccttctctgcagcggacaatgatcagatatttcacatcgaatccagcctcaaaggtcctttgttctccaatgttgctgtcaacattcagtcacttctccttatttcacatggcagcaaatatgtatgttttgtggagcttctcttccagcatagtgaacattctgggtcaagagcagttcatggcagtgtacctatctgcaggtgttatttccaattttgtcagttacctgggtaaagttgccacaggaagatatggaccatcacttggtgcatctggtgccatcatgacagtcctcgcagctgtctgcactaagatcccagaagggaggcttgccattattttccttccgatgttcacgttcacagcagggaatgccctgaaagccattatcgccatggatacagcaggaatgatcctgggatggaaattttttgatcatgcggcacatcttgggggagctctttttggaatatggtatgttacttacggtcatgaactgatttggaagaacagggagccgctagtgaaaatctggcatgaaataaggactaatggccccaaaaaaggaggtggctctaagtaa

SEQ ID NO: 5 - DNA, 47 bp, Artificial Sequence
  Description of Artificial Sequence Synthetic oligonucleotide
ccaacttggagcttctagtaagttctctactagaagctccaagttgg

SEQ ID NO: 6 - DNA, 28 bp, Artificial Sequence
  Description of Artificial Sequence Synthetic primer
ggcttcctagggtttatcgtgtgagcac

SEQ ID NO: 7 - DNA, 30 bp, Artificial Sequence
  Description of Artificial Sequence Synthetic primer
ggccacctacggtgaaaagaaagatgaagc

SEQ ID NO: 8 - DNA, 21 bp, Artificial Sequence
  Description of Artificial Sequence Synthetic oligonucleotide
aaatccagggtccagagttat

SEQ ID NO: 9 - protein, 69 aa, Homo sapiens
CKGPCQRVGPGKLRVQCSTCRQATLTLTQGPSCWDDVLIPNRMSGECQSPHCPGTSAEFFFKCGAHPTS

SEQ ID NO: 10 - protein, 69 aa, Danio rerio
CKTVCKAIQPGKLRVRCKDCKQGTLTLSRGPSCWDDVLLPNRIHGVCQSQGCNGRLAEFYLKCASHPTC

SEQ ID NO: 11 - protein, 76 aa, Drosophila sp.
CSQCDKLCNGKLRVRCALCKGGAFTVHRDPECWDDVLKSRRIPGHCESLEVACVDNAAGDPPFAEFFFKCAEHVSG

SEQ ID NO: 12 - protein, 70 aa, Unknown
  Description of Unknown Parkin sea urchin polypeptide
CKSHCRSVQPGKLRVCCQTCKDNAFIVKEDPVCWDDVILSNRISGSCFVPGCQGQKAEFFFKCSSHASSV

SEQ ID NO: 13 - protein, 68 aa, Caenorhabditis elegans
CKNCDDVKRGKLRVYCQKCSSTSVLVKSEPQNWSDVLKSKRIPAVCEECCTPGLFAEFKFKCLACNDP

SEQ ID NO: 14 - protein, 69 aa, Unknown
  Description of Unknown Parkin sea snail polypeptide
CKDCRGLNPGKLRFNCSTCKEGAFITDRGPDSWYDITVPNRISGNCQNQTCDGKMAEFYFKCGESHNDI

SEQ ID NO: 15 - protein, 66 aa, Trichoplax adhaerens
CKRCGTVTPGKLRARCSKCKESSVTLVRGPESWDDILIYGRIKGTCQFECSNTIVEFYFKCGSHLA

SEQ ID NO: 16 - protein, 18 aa, Homo sapiens
EGIPPDQQRLIFAGKQLE

SEQ ID NO: 17 - protein, 18 aa, Homo sapiens
QGVPADQLRVIFAGKELR

SEQ ID NO: 18 - protein, 18 aa, Danio rerio
QGVQSDQLRVIFAGRELC

SEQ ID NO: 19 - protein, 18 aa, Drosophila sp.
LGLQPDDLKIIFAGKELS

SEQ ID NO: 20 - protein, 18 aa, Unknown
  Description of Unknown Parkin sea urchin peptide
SGQSPADIRLVFAGKLID

SEQ ID NO: 21 - protein, 18 aa, Caenorhabditis elegans
TEIPSDELEVVFCGKKLS

SEQ ID NO: 22 - protein, 18 aa, Unknown
  Description of Unknown Parkin sea snail peptide
DDICEEELRFIIGGNILD

SEQ ID NO: 23 - protein, 20 aa, Homo sapiens
GCAGPCGRAVFLAFGLGLGL

SEQ ID NO: 24 - protein, 58 aa, Homo sapiens
FVVRAWGCAGPCGRAVFLAFGLGLGLIEEKQAESRRAVSACQEIQAIFTQKSKPGPDP

SEQ ID NO: 25 - protein, 58 aa, Bos taurus
FVVRARGGAGPCGRAVFLAFGLGLGLIEEKQAEGRRAASACEEIQAIFTQKNKLLPDP

SEQ ID NO: 26 - protein, 58 aa, Mus musculus
FMVRARGGAGPCGRAVFLAFGLGLGLIEEKQAEGRRAASACQEIQAIFTQKTKRVSDP

SEQ ID NO: 27 - protein, 58 aa, Rattus norvegicus
FVVRARGGAGPCGRAVFLAFGLGLGLIEEKQAESRRAASACQEIQAIFTQKNKQVSDP

SEQ ID NO: 28 - protein, 59 aa, Danio rerio
AFRRVIGGGSARNRAVFLAFGVGLGLIEQEQEEDRTSAALCQEIQAVFRKKKPQSLPKP

SEQ ID NO: 29 - protein, 60 aa, Drosophila melanogaster
LRQRATRKLFFGDSAPFFALIGVSLASGSGVLSKEDELEGVCWEIREAASRLQNAWNHDE

(19) United States
(12) Patent Application Publication
(10) Pub. No.: US 2012/0277286 A1
(43) Pub. Date: Nov. 1, 2012

(54) COMPOSITIONS AND METHODS FOR THE TREATMENT OR PREVENTION OF MITOCHONDRIAL DISEASES

(75) Inventors: Richard J. Youle, Bethesda, MD (US); Derek Narendra, Washington, DC (US); Der-Fen Suen, Taipei (TW)

(73) Assignee: The Government of the USA, as represented by the Secretary, Department of Health & Human Services, Bethesda, MD (US)

(21) Appl. No.: 13/460,852

(22) Filed: May 1, 2012

Related U.S. Application Data
(63) Continuation-in-part of application No. PCT/US2010/054802, filed on Oct. 29, 2010.
(60) Provisional application No. 61/256,601, filed on Oct. 30, 2009.

(51) Int. Cl. A61K 31/7088; A61K 31/713; C12N 5/075; C12Q 1/02; C12N 5/071; C12N 5/0793

(52) U.S. Cl. 514/44 A; 435/375; 514/44 R; 435/29

(57) ABSTRACT

The present invention features compositions and methods for the treatment or prevention of diseases associated with a mitochondrial defect.

### CROSS-REFERENCE TO RELATED APPLICATION

This application is a continuation-in-part application of International Application No. PCT/US2010/054802, filed Oct. 29, 2010, which claims the benefit of U.S. Provisional Application No. 61/256,601, filed Oct. 30, 2009. The contents of each of these applications are incorporated herein by reference in their entirety.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. The Government has certain rights in this invention.

### BACKGROUND OF THE INVENTION

Mitochondrial DNA (mtDNA) mutations are responsible for a number of severe syndromes, with symptoms ranging from epilepsy and encephalopathy to lactic acidosis and diabetes. In addition, somatically acquired mtDNA mutations have been linked to the pathogenesis of common diseases, such as cancer, diabetes mellitus, and neurodegenerative disorders. For example, patients with sporadic Parkinson's disease have a greater number of functionally deleterious mtDNA mutations in their substantia nigral neurons compared to age matched controls, and increased mtDNA deletions, as is observed in patients with multiple mtDNA deletion syndromes, appears to be sufficient to cause parkinsonism.

A typical cell contains thousands of copies of mtDNA, and an electrochemically discrete mitochondrion may contain zero to hundreds of copies of the mitochondrial genome depending on the interconnectivity of the mitochondrial network. Within the cells of a patient affected with a mitochondrial disease, mutated mtDNA typically coexists with wild-type mtDNA. In this heteroplasmic state, wild-type and mutant mtDNA are packed in separate nucleoids and rarely mix even though nucleoids move relatively freely in mitochondria. The severity of cellular dysfunction and disease caused by a given mtDNA mutation depends on the ratio of mutant mtDNA to wild-type mtDNA in the cell. Compositions and methods for treating or preventing diseases associated with mitochondrial defects (e.g., mutations, dysfunction) are urgently required.

### SUMMARY OF THE INVENTION

As described below, the present invention features compositions and methods for the treatment or prevention of diseases associated with a mitochondrial defect.

In one aspect, the invention generally provides a method of reducing the number of defective mitochondria in a cell, the method involving contacting the cell with an agent that increases Pink1 or Parkin expression or biological activity in the cell, thereby reducing the number of defective mitochondria in the cell.

In another aspect, the invention provides a method of selectively eliminating from a cell a mitochondria having a mutation in mitochondrial DNA, the method comprising contacting the cell with a mammalian expression vector encoding a Parkin or PINK1 polypeptide or fragment thereof, and increasing mitophagy of said mitochondria.

In yet another aspect, the invention provides a method of treating or preventing a mitochondrial disease in a subject, the method comprising administering to the subject an effective amount of an agent that increases Pink1 or Parkin expression or biological activity in a cell, thereby treating the disease.

In still another aspect, the invention provides a method of treating or preventing a mitochondrial disease in a subject, the method comprising administering to the subject an effective amount of a mammalian expression vector encoding a Parkin or PINK1 polypeptide or fragment thereof, and selectively eliminating from the subject a mitochondria having a mutation in mitochondrial DNA, thereby treating or preventing the disease.

In another aspect, the invention provides a method of selecting a subject as having a disease or disorder characterized by mitochondrial dysfunction, involving determining the presence of defective mitochondria in a cell of the subject, administering a therapeutically effective amount of a Parkin or PINK1 polypeptide to the subject; and determining an increase in mitochondrial function or a decrease in the number of defective mitochondria in a cell of the subject.

In still another aspect, the invention provides a kit for treating a mitochondrial disease comprising a pharmaceutical composition comprising an effective amount of a Parkin or PINK1, instructions for identifying a subject in need of such treatment, and directions for administering the pharmaceutical composition to the subject.

In another aspect, the invention provides a method for identifying a compound useful for the treatment of a mitochondrial disease, the method comprising contacting a cell with a compound and an agent that disrupts mitochondrial function; and identifying a reduction in the number of defective mitochondria in the cell relative to a control cell not contacted with the candidate compound, wherein a compound that reduces the number of defective mitochondria in the cell is identified as useful for the treatment of a mitochondrial disease.

In another aspect, the invention provides methods for identifying a compound useful for the treatment of a mitochondrial disease, the method comprising contacting a cell with a compound and an agent that disrupts mitochondrial function; and identifying an increase of PINK1 or Parkin associated with mitochondria in the cell relative to a control cell not contacted with the candidate compound, wherein a compound that increases PINK1 or Parkin association with mitochondria in the cell is identified as useful for the treatment of a mitochondrial disease.

In another aspect, the invention provides a method for identifying a compound useful for the treatment of a mitochondrial disease, the method comprising contacting a cell comprising a mutation in mitochondrial DNA with a compound; and identifying a reduction in the number of defective mitochondria in the cell relative to a control cell not contacted with the candidate compound, wherein a compound that reduces the number of defective mitochondria in the cell is identified as useful for the treatment of a mitochondrial disease.

In another aspect, the invention provides methods for identifying a compound useful for the treatment of a mitochondrial disease, the method comprising contacting a cell with a compound; and identifying an increase of PINK1 or Parkin associated with mitochondria in the cell relative to a control cell not contacted with the candidate compound, wherein a compound that increases PINK1 or Parkin association with mitochondria in the cell is identified as useful for the treatment of a mitochondrial disease.

In still another aspect, the invention provides a method for identifying a compound useful for the treatment of Parkinson's disease, the method comprising contacting a dopaminergic cell with a candidate compound and an agent that disrupts mitochondrial function; and identifying a reduction in the number of defective mitochondria in the cell relative to a control cell not contacted with the candidate compound, wherein a compound that reduces the number of defective mitochondria in the cell is identified as useful for the treatment of a Parkinson's disease.

In a related aspect, the invention provides a method for identifying a compound useful for the treatment of Parkinson's disease, the method comprising contacting a cell comprising a mutation in Pink1 or Parkin with a candidate compound; and identifying a reduction in the number of defective mitochondria in the cell relative to a control cell not contacted with the candidate compound, wherein a compound that reduces the number of defective mitochondria in the cell is identified as useful for the treatment of a Parkinson's disease.

In various embodiments of the two previous aspects, the increase in expression is detected at the level of transcription or at the level of translation.

In another aspect, the invention provides a method for identifying a compound useful for the treatment of a subject having a mitochondrial disease, the method involving contacting a cell derived from the subject with a compound; and identifying a reduction in the number of defective mitochondria in the cell relative to a control cell not contacted with the candidate compound, wherein a compound that reduces the number of defective mitochondria in the cell is identified as useful for the treatment of said mitochondrial disease in the subject.

In another aspect, the invention provides a method for identifying a compound useful for the treatment of a subject having a mitochondrial disease, the method involving contacting a cell derived from the subject with a compound and an agent that disrupts mitochondrial function; and identifying a reduction in the number of defective mitochondria in the cell relative to a control cell not contacted with the candidate compound, wherein a compound that reduces the number of defective mitochondria in the cell is identified as useful for the treatment of the subject having mitochondrial disease.

In another aspect, the invention provides a method for ameliorating Parkinson's disease in a subject, the method comprising administering to the subject an agent that reduces the biological activity or expression of PARL. In one embodiment, the agent is an inhibitory nucleic acid molecule (e.g., siRNA, shRNA or antisense polynucleotide) that reduces the expression of PARL polynucleotide or polypeptide. In another embodiment, the agent is a protease inhibitor that reduces PARL proteolytic activity.

In various embodiments of any of the above aspects or any other aspect of the invention, the agent is a polypeptide, polynucleotide, small chemical compound, or microRNA. In other embodiments of the above aspects, the cell (e.g., mammalian, human, rodent cell) is an ocular cell, neuron, muscle cell, or oocyte. In still other embodiments, the agent increases (e.g., by at least about 10%, 25%, 50%, 75%, or more) levels of a Pink1 polypeptide or Pink1 polynucleotide or increases (e.g., by at least about 10%, 25%, 50%, 75%, or more) levels of a Parkin polypeptide or polynucleotide. In yet another embodiment of the above aspects, the agent is an expression vector encoding a Pink1 or Parkin polynucleotide. In yet another embodiment, the method increases biogenesis of new mitochondria. In still other embodiments, a defective mitochondria has a dysfunction that is any one or more of a reduction in the activity of a mitochondrial enzyme, reduced electron transport chain (ETC) activity, diminished membrane potential, increased reactive oxygen species production, mitochondrial fragmentation, calcium dysregulation, and a mutation in mitochondrial DNA (mtDNA) (e.g., a Parkin mutation selected from the group consisting of Q311X, K211N, C212Y, C253Y, C289G, C441R, I44A, R42P, A46P, and R275W or a Pink1 mutation that is A168P, H271Q, G309D, L347P or G411S). In various embodiments of the above aspects or any other aspect of the invention delineated herein, the cell is a human cell in vitro, ex vivo, or in vivo. In still other embodiments, the disease is associated with a mitochondrial dysfunction selected from the group consisting of a reduction in the activity of a mitochondrial enzyme, reduced electron transport chain (ETC) activity, diminished membrane potential, increased reactive oxygen species production, mitochondrial fragmentation, calcium dysregulation, and a mutation in mitochondrial DNA (mtDNA). In still other embodiments, the disease is a mitochondrial disease (e.g., Neurogenic muscular weakness-Ataxia-Retinitis pigmentosa (NARP), Multiple Sclerosis-like Syndrome (MSS); Maternally Inherited CardioMyopathy (MCIM); Progressive External Ophthalmoplegia (PEO); Myoclonic Epilepsy with Ragged-Red Fibers (MERRF); Myoneurogastrointestinal disorder and encephalopathy (MNGIE), Pearson Marrow syndrome, Kearns-Sayre-CPEO, Leber hereditary optic neuropathy (LHON), Aminoglycoside-associated deafness, Diabetes with deafness, Luft disease, Leigh syndrome (Complex I, COX, PDH), Alpers Disease, MCAD, SCAD, SCHAD, VLCAD, LCHAD, Glutaric aciduria II, and Lethal infantile cardiomyopathy). In still other embodiments, the disease is cancer, diabetes mellitus, or sporadic Parkinson's disease. In still other embodiments, the method increases autophagy of small defective mitochondria that lack membrane potential and/or increases biogenesis of new mitochondria. In other embodiments of the above aspects, the subject is a human subject diagnosed as having mitochondrial dysfunction. In one embodiment, the diagnosis involves a muscle biopsy or EEG. In still another embodiment, the agent reduces defective mitochondria by at least about 15-25%, by at least about 50-75% or by about 100%. In still other embodiments, the Parkin or Pink1 polypeptide is a fragment comprising at least about 75 to 150 amino acids. In yet another embodiment, the subject is a mammal (e.g., human). In still another embodiment, a nucleic acid encoding a Parkin or Pink1 polypeptide is under the control of a heterologous promoter (e.g., the Nrf promoter). In still another embodiment, the expression construct is a viral or non-viral expression construct. In still another embodiment, the viral expression construct is adenovirus, retrovirus, adeno-associated virus, herpesvirus, vaccinia virus or polyoma virus.

The invention provides compositions and methods for the treatment or prevention of diseases associated with a mitochondrial defect. Compositions and articles defined by the invention were isolated or otherwise manufactured in connection with the examples provided below. Other features and advantages of the invention will be apparent from the detailed description, and from the claims.

### DEFINITIONS

By “Pink1 polypeptide” is meant a protein or fragment thereof having at least 85% amino acid sequence identity to GenBank Accession No. AAQ89316 and having Pink1 biological activity. An exemplary Pink1 polypeptide sequence is provided below:

```
    1 mavrqalgrg lqlgralllr ftgkpgrayg lgrpgpaagc vrgerpgwaa gpgaeprrvg
  61 lglpnrlrff rqsvaglaar lqrqfvvraw gcagpcgrav flafglglgl ieekqaesrr
121 aysacqeiqa iftqkskpgp dpldtrrlqg frleeyligq sigkgcsaav yeatmptlpq
181 nlevtkstgl lpgrgpgtsa pgegqerapg apafplaikm mwnisagsss eailntmsqe
241 lvpasrvala geygavtyrk skrgpkqlap hpniirvlra ftssvpllpg alvdypdvlp
301 srlhpeglgh grtlflvmkn ypctlrqylc vntpsprlaa mmllqllegv dhlvqqgiah
361 rdlksdnilv eldpdgcpwl viadfgccla desiglqlpf sswyvdrggn gclmapevst
421 arpgpravid yskadawavg aiayeifglv npfygqgkah lesrsyqeaq lpalpesvpp
481 dvrqlvrall qreaskrpsa rvaanvlhls lwgehilalk nlkldkmvgw llqqsaatll
541 anrltekccv etkmkmlfla nleceticqa alllcswraa l
```

By “Pink1 polynucleotide” is meant a nucleic acid molecule encoding a Pink1 polypeptide.

By “Pink1 biological activity” is meant Parkin recruitment, serine/threonine kinase activity, or any other biological activity required for mitochondrial function.

By “Parkin polypeptide” is meant a protein or fragment thereof having at least 85% amino acid sequence identity to GenBank Accession No. BAA25751 and having Parkin biological activity. An exemplary Parkin polypeptide sequence is provided below:

```
    1 mivfvrfnss hgfpvevdsd tsifqlkevv akrqgvpadq lrvifagkel rndwtvqncd
  61 ldqqsivhiv grpwrkgqem natggddprn aaggcerepq sltrvdlsss vlpgdsvgla
121 vilhtdsrkd sppagspagr siynsfyvyc kgpcqrvqpg klrvqcstcr qatltltqgp
181 scwddvlipn rmsgecqsph cpgtsaefff kcgahptsdk etpvalhlia tnsrnitcit
241 ctdvrspvlv fqcnsrhvic ldcfhlycvt rlndrqfvhd pqlgyslpcv agcpnslike
301 lhhfrilgee gynryqqyga eecvlqmggv lcprpgcgag llpepdgrkv tceggnglgc
361 gfafcrecke ayhegecsav feasgtttqa yrvderaaeq arweaasket ikkttkpcpr
421 chvpvekngg cmhmkcpqpq crlewcwncg cewnrvcmgd hwfdv
```

By “Parkin polynucleotide” is meant a nucleic acid molecule encoding a Parkin polypeptide.

By “Parkin biological activity” is meant binding to Pink1, ubiquitin ligase activity, binding to mitochondria or any other Parkin biological activity required for mitochondrial maintenance or function.

By “PARL polypeptide” is meant a polypeptide or fragment thereof having at least 85% amino acid identity to GenBank Accession No. Q9H300.2 and having proteolytic activity. An exemplary PARL polypeptide sequence is provided below:

```
    1 mawrgwaqrg wgcgqawgas vggrsceelt avltppqllg rrfnffiqqk cgfrkaprkv
  61 eprrsdpgts geaykrsali ppveetvfyp spypirslik plfftvgftg cafgsaaiwq
121 yeslksrvqs yfdgikadwl dsirpqkegd frkeinkwwn nlsdgqrtvt giiaanvlvf
181 clwrvpslqr tmiryftsnp askvlcspml lstfshfslf hmaanmyvlw sfsssivnil
241 ggegfmavyl sagvisnfvs yvgkvatgry gpslgasgai mtvlaavctk ipegrlaiif
301 lpmftftagn alkaiiamdt agmilgwkff dhaahlggal fgiwyvtygh eliwknrepl
361 vkiwheirtn gpkkgggsk
```

By “PARL polynucleotide” is meant a nucleic acid molecule encoding a PARL polypeptide. The sequence of an exemplary PARL polynucleotide is provided below:

```
      1 atggcgtggc gaggctgggc gcagagaggc tggggctgcg gccaggcgtg gggtgcgtcg
    61 gtgggcggcc gcagctgcga ggagctcact gcggtcctaa ccccgccgca gctcctcgga
  121 cgcaggttta acttctttat tcaacaaaaa tgcggattca gaaaagcacc caggaaggtt
  181 gaacctcgaa gatcagaccc agggacaagt ggtgaagcat acaagagaag tgctttgatt
  241 cctcctgtgg aagaaacagt cttttatcct tctccctatc ctataaggag tctcataaaa
  301 cctttatttt ttactgttgg gtttacaggc tgtgcatttg gatcagctgc tatttggcaa
  361 tatgaatcac tgaaatccag ggtccagagt tattttgatg gtataaaagc tgattggttg
  421 gatagcataa gaccacaaaa agaaggagac ttcagaaagg agattaacaa gtggtggaat
  481 aacctaagtg atggccagcg gactgtgaca ggtattatag ctgcaaatgt ccttgtattc
  541 tgtttatgga gagtaccttc tctgcagcgg acaatgatca gatatttcac atcgaatcca
  601 gcctcaaagg tcctttgttc tccaatgttg ctgtcaacat tcagtcactt ctccttattt
  661 cacatggcag caaatatgta tgttttgtgg agcttctctt ccagcatagt gaacattctg
  721 ggtcaagagc agttcatggc agtgtaccta tctgcaggtg ttatttccaa ttttgtcagt
  781 tacctgggta aagttgccac aggaagatat ggaccatcac ttggtgcatc tggtgccatc
  841 atgacagtcc tcgcagctgt ctgcactaag atcccagaag ggaggcttgc cattattttc
  901 cttccgatgt tcacgttcac agcagggaat gccctgaaag ccattatcgc catggataca
  961 gcaggaatga tcctgggatg gaaatttttt gatcatgcgg cacatcttgg gggagctctt
1021 tttggaatat ggtatgttac ttacggtcat gaactgattt ggaagaacag ggagccgcta
1081 gtgaaaatct ggcatgaaat aaggactaat ggccccaaaa aaggaggtgg ctctaagtaa
```

By “PARL biological activity” is meant proteolytic activity.

By “biogenesis of new mitochondria” is meant the production of mitochondria in a cell.

By “defective mitochondria” is meant mitochondria having a mutation or deletion in mitochondrial DNA or any other alteration that results in a reduction in mitochondrial function. Exemplary defects associated with mitochondrial dysfunction include but are not limited to reductions in the activity of a mitochondrial enzyme such as cytochrome oxidase, reduced electron transport chain (ETC) activity, diminished membrane potential, increased reactive oxygen species production, mitochondrial fragmentation, or calcium dysregulation.

By “selectively eliminate dysfunctional mitochondria” is meant specifically reducing the number of defective mitochondria without having a deleterious effect on normal mitochondria. In one embodiment, the selective elimination of defective mitochondria is associated with the biogenesis of new mitochondria.

By “mutation in mitochondrial DNA” is meant any alteration in the sequence of a mitochondrial gene relative to a wild-type reference gene.

By “mitochondrial disease” is meant any pathological condition associated with an increase in the number of defective mitochondria or a reduction in mitochondrial function.

Such diseases may be hereditary or somatic. In fact, many mitochondrial mutation diseases result from sporadic/somatic mutations. In one embodiment, a Parkinson's disease is specifically excluded from the definition of a mitochondrial disease.

By “hereditary mitochondrial disease” is meant a disease or condition associated with a genetic mutation in a mitochondrial gene. and not hereditary.

By “defective mitochondria” is meant a mitochondrion having a genetic mutation or a reduction in mitochondrial function.

By “mitochondrial dysfunction” is meant any adverse change in mitochondrial activity.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “at risk of” is meant having a propensity to develop a disease or disorder. For example, a subject having a genetic mutation in a gene associated with a disease is at increased risk of developing the disease relative to a normal control subject.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “diagnosis” or “identifying a subject having” refers to a process of determining whether an individual is afflicted with a disease or has a genetic predisposition to develop a disease or disorder.

By “disease” is meant any condition or disorder that damages or interferes with the normal function of a cell, tissue, or organ. Examples of diseases associated with a mitochondrial defect (e.g., a mitochondrial mutation, mitochondrial dysfunction, or reduction in mitochondrial function) include NARP—neurogenic muscular weakness, ataxia, retinitis pigmentosa, MSS—multiple sclerosis-like syndrome; MCIM—maternally inherited cardiomyopathy; PEO—progressive external ophthalmoplegia; MERRF—myoclonic epilepsy with ragged-red fibers; Myoneurogastrointestinal disorder and encephalopathy (MNGIE), Pearson Marrow syndrome, Kearns-Sayre-CPEO, Leber hereditary optic neuropathy (LHON), Aminoglycoside-associated deafness, Diabetes with deafness, Luft disease, Leigh syndrome (Complex I, COX, PDH), Alpers Disease, MCAD, SCAD, SCHAD, VLCAD, LCHAD, Glutaric aciduria II, and Lethal infantile cardiomyopathy. MELAS, MILS. In addition, somatically acquired mtDNA mutations have been linked to the pathogenesis of common diseases, such as cancer, diabetes mellitus, autism and neurodegenerative disorders including Parkinson's disease.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 15%, 25%, 50%, 75%, or 100%.

By “inhibitory nucleic acid” is meant a double-stranded RNA, siRNA, shRNA, or antisense RNA, or a portion thereof, or a mimetic thereof, that when administered to a cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “subject” is meant a mammal, including, but not limited to, a human or non-human mammal, such as a bovine, equine, canine, ovine, or feline.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1H show that Parkin accumulates on impaired mitochondria. FIGS. 1A and 1B are micrographs of HEK293 cells treated with DMSO control (a) or 10 μM CCCP (b) for 1 hour, then immunostained for endogenous Parkin (green) and a mitochondrial marker, Tom20 (red). The bottom panels show enlarged views of the boxed areas. Arrows indicate mitochondria that colocalize with endogenous Parkin. FIGS. 1C and 1D are Western blots. HEK293 cells (FIG. 1C) and rat cortical neurons (FIG. 1D) were depolarized with CCCP for 1 and 5 hours, respectively. Cells were immunoblotted for endogenous Parkin. PNS, HM, and PHM indicate postnuclear supernatant, mitochondrial-rich heavy membrane pellet, and post-heavy membrane supernatant, respectively. VDAC is a mitochondrial marker. FIG. 1E shows six micrographs. HeLa cells expressing YFP-Parkin (green) were treated with DMSO, 10 μM CCCP, or 10 μM CCCP+10 μM oligomycin for 1 hour. Cells were stained for the mitochondrial marker cytochrome c (red). Line scans below the images indicate colocalization between Parkin (green) and mitochondria (red) and correlate to the lines drawn in the images. FIG. 1F is a graph showing YFP-Parkin colocalization with mitochondria scored for ≧300 cells per condition in at least two experiments. FIG. 1G is a Western blot showing YFP-Parkin accumulation in mitochondrial fraction assessed as in panel FIG. 1c. Numbers to the right of the gel blots indicate molecular weight standards in kD. FIG. 1H is a graph showing HeLa cells treated with 2 mM paraquat or paraquat +10 mM N-acetyl-cysteine (NAC) for 24 hours scored for colocalization, as in panel f. Error bars indicate standard deviation of at least three replicates.

FIG. 1I-a, 1I-b, and 1I-c show that Paraquat triggers Parkin recruitment to mitochondria, and Parkin recruitment to depolarized mitochondria is not blocked by antioxidant. FIG. 1I-A includes a series of eight micrographs. HeLa cells expressing YFP-Parkin (green) treated with media control, 10 mM N-acetyl-cysteine (NAC), 2 mM paraquat, or paraquat +NAC for 24 h. Cells were immunostained for Tom20 (red). FIG. 1I-B includes eight micrographs showing HeLa cells expressing YFP-Parkin (green) treated with DMSO, 10 μM NAC, 10 μM CCCP, or CCCP+NAC for 1 hour. Cells were immunostained for Tom20 (red). Bars, 10 μm. FIG. 1I-C is a graph that quantitates YFP-Parkin colocalization with mitochondria scored (greater than 150 cells in at least three experiments). Error bars indicate standard deviation of at least three replicates.

FIG. 1J-a and 1Jb are micrographs showing that Parkin recruited to depolarized mitochondria in paraquat-treated cells, and Parkin has cytosolic distribution in Mfn1−/− and Mfn2−/− knockout single MEFs. FIG. 1J-a shows HeLa cells coexpressing YFP-mito (pseudo-color red in merge) and ECFP-Parkin (blue in merge) treated with 2 mM paraquat for 24 hours and pulsed with the potentiometric dye MitoTracker red (pseudo-color green in merge). Parkin colocalizes with YFPmito but not MitoTracker red (arrows), which indicates recruitment to depolarized mitochondria. FIG. 1J-b Cytosolic distribution of YFP-Parkin (white) in Mfn1−/− and Mfn2−/− single knockout MEFs. Bars, 10 μm.

FIGS. 2A-2G show results of a FLIP analysis of Parkin diffusibility and selectivity of Parkin accumulation. FIG. 2A-2C includes fluorescent micrographs and graphs showing a FLIP analysis with quantification after treatment with DMSO (FIG. 2A, top, and 2B) or CCCP (2A, bottom, and 2C; n≧3 in each treatment). Rectangles in panel 2A indicate the bleach ROI. Outlines demarcate the edges of cells expressing YFP-Parkin. FIG. 2D are micrographs showing YFP localization in WT and Mfn1−/−, Mfn2−/− double knockout MEF cells expressing YFP-Parkin. FIG. 2E is a graph showing YFP-Parkin scored for colocalization as in FIG. 1F. Error bars indicate standard deviation of at least three replicates. FIG. 2F includes five micrographs showing Mfn1−/−, Mfn2−/− double knockout MEF cells transfected with YFP-Parkin (green) and pulsed with the potentiometric dye MitoTracker (blue in merge) 15 minutes before fixation. Cells were immunostained for cytochrome c (red). A line scan of fluorescence through two Parkin-positive mitochondria depicts colocalization between Parkin, MitoTracker, and cytochrome c. The right four panels show an enlarged view of the boxed area. Arrows indicate mitochondria (identified by anti-cytochrome c) that were depolarized (as assessed by their failure to take up the dye MitoTracker; arrowheads represent mitochondria (identified by anti-cytochrome c) that were electrochemically active (as assessed by their ability to take up the dye MitoTracker). YFP-Parkin colocalizes with depolarized mitochondria (arrows), but not with electrochemically active mitochondria. FIG. 2g is a graph showing the mitochondrial volume for each Mfn1−/−, Mfn2−/− MEF cell was segregated into Parkin-positive and Parkin-negative subsets. Mean MitoTracker fluorescence intensity was measured for each subset (n=9). Bars: (a, d, and f, left) 5 μm; (f, right four panels) 1 μm.

FIG. 2H provides micrographs showing that Parkin-mediated mitophagy was blocked by lysosomal inhibitor, bafilomycin, and an inhibitor of autophagy, 3-methyladenine. YFP-Parkin-expressing HeLa cells were treated with DMSO, 10 μM CCCP, CCCP+10 mM 3-methyladenine (3-MA), or CCCP+100 nM Bafilomycin for 24 hours. Outlines demarcate the edges of cells expressing YFP-Parkin. Bar, 10 μm.

FIGS. 3A-3D show that mitochondrial fragmentation does not induce Parkin accumulation independently of mitochondrial membrane potential. FIGS. 3A, 3B, and 3C are micrographs. FIG. 3D is a graph. HeLa cells were cotransfected with YFP-Parkin (green) and with empty vector (FIG. 3A), vMIA (FIG. 3B), or Drp1 K38A (FIG. 3C). Cells were treated with 10 μM CCCP (FIG. 3A, right, and FIG. 3C) or DMSO (FIG. 3A, left, and FIG. 3B) for 1 hour. Mitochondria were immunostained for cytochrome c (red). The bottom two panels in each column show an enlarged view of the boxed regions. FIG. 3D quantitates YFP-Parkin colocalization with mitochondria scored as in FIG. 1F. Error bars indicate standard deviation of at least three replicates. Bars, 5 μm

FIGS. 4A-4H show selective mitochondrial elimination by Parkin under depolarizing conditions. FIGS. 4A-4C are micrographs. FIGS. 4A and 4B show HeLa cells expressing YFP-Parkin (green) incubated for 12 hours (4A) or 48 hours (4B, left) with 10 μM CCCP. Cells were immunostained for Tom20 (red). Parkin-expressing HeLa cells display less mitochondrial mass compared with surrounding cells at 12 hours and complete loss of mitochondria by 48 h. FIG. 4B shows a similar loss of mitochondria observed with anti-cytochrome c (red, middle) and anti-TRAP1 (red, right) antibodies. FIG. 4C shows no loss of peroxisomes immunostained for PMP70 (red) in YFP-Parkin-transfected cells relative to surrounding untransfected cells. Outlines demarcate the edges of cells expressing YFP-Parkin. Bars, 10 μm. FIG. 4D-4E are micrographs obtained using electron microscopy of untransfected HeLa cells (FIG. 4D) or HeLa cells expressing YFP-Parkin (FIG. 4E) and treated with 10 μM CCCP for 48 hours. Many mitochondria and few lysosomes were observed in control cells, and no mitochondria and many lysosomes were observed in YFP-Parkin-transfected cells. Bars, 500 nm. FIG. 4F is a graph that quantitates the number of mitochondria and late lysosomes/μm² of cytoplasm in 22 randomly selected cells per condition. FIG. 4G is a graph that shows the number of PMP70-stained peroxisomes per cell in YFP-Parkin-transfected and untransfected cells (n=5). Error bars indicate standard deviation of at least three replicates. FIG. 4H is a graph that shows results in control HeLa cells or HeLa cells transfected with YFP-Parkin treated with 10 μM CCCP for 72 hours (day 0) and cultured in glucose or galactose media for 1-4 days. Cells were fixed and stained for Tom20 and Hoechst33342 (nuclei). Cells with nonapoptotic nuclei in a representative area of the slide on days 0-4 were counted and represented in the graph as a percentage of nonapoptotic cells on day 0 (≧160 cells per condition on day 0 in at least two experiments).

FIGS. 5A-5F show mitophagy induced by Parkin. FIG. 5A provides ten fluorescent micrographs of HeLa cells stably expressing GFP-LC3 (green) transfected with mCherry-Parkin (not depicted) and treated with 10 μM CCCP for 1 hour. Parkin-negative cells (left) display less overlap between autophagosomes and mitochondria (red) than Parkin-positive cells (right), as assessed by (FIG. 5B) counting the number of mitochondria encapsulated by LC3-positive autophagosomes in >30 cells per condition in at least three independent experiments. FIG. 5C provides micrographs of HeLa cells stably expressing GFP-LC3 (green) and transiently transfected with mCherry-Parkin (white) were immunostained for cytochrome c (red) to reveal colocalization of LC3, Parkin, and mitochondria after 1 hours exposure to CCCP. Arrows indicate mitochondria that colocalize with both mCherry-Parkin and GFP-LC3. Insets show an enlarged view of the boxed areas. (d) YFP-Parkin (green)-induced mitochondrial removal after 24 hours of CCCP (10 μM) exposure observed in WT MEFs (left) failed to occur in ATG5−/− MEFs (right) quantified (FIG. 5E) in ≧150 cells in at least three experiments. Cells were stained for Tom20 (red). Outlines demarcate the edges of cells expressing YFP-Parkin. FIG. 5f shows that 3-methyladenine (3MA) and bafilomycin blocked Parkin-induced mitophagy in HeLa cells quantified as in panel E. Error bars indicate standard deviation of at least three replicates. Bars: (c and d) 10 μm; (a and c, insets) 1 μm.

FIGS. 6A-6J show that PINK1 selectively accumulates on depolarized mitochondria. FIG. 6A shows two Western blots. HeLa cells stably expressing YFP-Parkin were treated with 10 μM CCCP in serum at time point 0, fractionated, and carbonate extracted. The carbonate extracted pellet, which is enriched in integral mitochondrial proteins, was run on SDS gels and immunoblotted for endogenous PINK1 and the mitochondrial protein VDAC. HeLa cells stably expressing YFP-Parkin were used in the initial experiments because it was unclear whether the stability of PINK1 would be affected by the absence of Parkin, as has been reported previously (Abou-Sleiman P M, Muqit M M, McDonald N Q, Yang Y X, Gandhi S, et al. (2006) Ann Neurol 60: 414-419). FIG. 6B shows two Western blots. M17 human neuroblastoma cells stably transduced with control shRNA or PINK1 shRNA were treated with 20 μM CCCP in serum and fractionated. The mitochondria-rich membrane fraction was run on SDS gels and immunoblotted as in FIG. 6A. FIG. 6C shows two Western blots. E18 rat cortical neurons (7 days in vitro) were transfected with PINK1-V5. The next day the cells were treated with 1 μM CCCP for 6 hours. Whole cell lysates were run on SDS page gels and immunoblotted as in FIG. 6A. FIG. 6D shows fluorescent images obtained from live cell imaging of HeLa cells transfected with PINK1-YFP (green). The cells were treated with 10 μM CCCP in serum at time point 0. Mitochondria were labeled by pulsing with Mitotracker Red (MTR) (red) before depolarization with CCCP. FIG. 6E provides three micrographs. Mfn1/2 null MEFs transfected with PINK1-YFP (green). All mitochondria were stained with antibody against cytochrome c (white) and bioenergetically coupled mitochondria were stained by pulsing cells with Mitotracker Red (MTR) (red). FIG. 6F is a graph that quantitates the average MTR intensity/pixel for PINK1 negative mitochondria and PINK1 positive mitochondria, respectively, measured in ≧8 cells in 2 independent experiments. Data from a representative experiment is shown. FIG. 6G includes four micrographs. HeLa cells were transfected with PINK1-YFP (green) and treated for 16 hours with 2 mM paraquat. Cells were pulsed with MTR (red), fixed, and immunostained for cytochrome c (white). FIG. 6H is a graph that represents the pearson coefficient indexes between PINK1-YFP intensity and cytochrome c intensity and PINK1-YFP intensity and MTR intensity, which were determined for ≧8 cells in 2 independent experiments. Data from a representative experiment is shown. FIG. 6I provides four micrographs. HeLa cells transfected with CFP-Parkin (green) and PINK1 KD-YFP (red) and treated for 16 hours with 2 mM paraquat. Cells were pulsed with MTR (white) and fixed. FIG. 6J is a graph showing the pearson coefficient indexes between PINK1 KD-YFP intensity and CFP-Parkin intensity and PINK1 KD-YFP intensity and MTR intensity, which were determined for ≧7 cells in 2 independent experiments. Data from a representative experiment is shown.

FIGS. 7A-7E show that Mitochondrial PINK1 accumulates on the outer mitochondrial membrane following mitochondrial depolarization. FIG. 7A shows two Western blots. HeLa cells treated with 1 μM of valinomycin without serum at time point 0 were fractionated, and carbonate extracted. The carbonate extracted pellet, which is enriched for proteins integral to mitochondria, was run on SDS gels and immunoblotted for endogenous PINK1 and mitochondrial protein VDAC. FIG. 7B shows three Western blots. HeLa cells stably expressing YFP-Parkin were treated with 2 μM of CCCP without serum at time point 0 and fractionated. The mitochondria-rich membrane fraction (lanes 1-2) and the cytosolic enriched post-membrane fraction (lanes 4-9) were run on SDS gels and immunoblotted for PINK1, tubulin, and VDAC. (C) PINK1^{-/-} MEFs transfected with PINK1-myc or left untransfected were treated with 2 μM CCCP without serum for 3 hours and fractionated. Mitochondrial rich membrane fraction was run on SDS gels and immunoblotted for PINK1 and VDAC. FIG. 7D shows two Western blots. HeLa cells transfected with PINK1-YFP or a kinase deficient version of PINK1 (PINK1 KD-YFP) were treated as in FIG. 7B. Whole cell lysates were run on SDS gels and immunoblotted for PINK1 and tubulin. Arrow indicates the predicted MW of full length PINK1-YFP. FIG. 7E shows two Western blots. HeLa cells stably expressing YFP-Parkin were treated with 10 μM CCCP for 3 hours and fractionated. The mitochondria-enriched membrane fraction was aliquoted. Each aliquot was treated with 0 to 100 μg/mL protease K and immunoblotted for endogenous PINK1, the outer membrane protein TOM20, the inner membrane protein Tim23, and matrix protein Hsp60.

FIGS. 8A-8C show that PINK1 accumulates following inhibition of voltage-sensitive cleavage. FIG. 8A includes two Western blots. HeLa cells stably expressing YFP-Parkin were treated with DMSO for 3.5 hours, 2 μM CCCP for 3.5 hours, or CCCP for 3 hours followed by washout of CCCP for 0.5 hours in the absence of serum. 50 μM MG132 and/or 100 μM cyclohexamide were added for the last 1 hr of treatment. Whole cell lysates (WCL) run on SDS gels and immunoblotted for endogenous PINK1 and tubulin. FIG. 8B is a model depicting the two-step processing of PINK1. FIG. 8C is a graph showing Pink1/β-actin mRNA measured using Quantitative RT-PCR. Q-RT-PCR was used to measure relative PINK1 mRNA expression in HeLa cells treated with DMSO or CCCP for 1 hr. The graph represents the results from 4 independent experiments. As a positive control relative PINK1 mRNA levels were also measured in HeLa cells following exogenous expression of PINK1. PINK1 mRNA expression levels were normalized to the housekeeping gene β-actin.

FIGS. 9A-9E show that PINK1 accumulates independently of PARL and Parkin expression. FIG. 9A includes three Western blots. HeLa cells co-transfected with PARL-Flag and either PARL shRNA or control shRNA were depolarized with 10 μM CCCP for 3 hours. Whole cell lysates were run on SDS gels and immunoblotted with antibodies against the N-terminus of PARL, the C-terminus of PARL, and Tubulin. FIG. 9B includes six Western blots. HeLa cells mock transfected or transfected with shRNA PARL were treated with DMSO or CCCP for 3 hours and fractionated. The mitochondria-enriched membrane fraction (left) and whole cell lysates (right) were run on SDS gels and immunoblotted for PINK1, the C-terminus of PARL, VDAC, and/or Tubulin. FIG. 9C includes four Western blots. Wild type or PARL null MEFs were transfected with PINK1-V5, treated as in FIG. 9B, and fractionated. The mitochondria-enriched heavy membrane fraction was run on SDS gels and immunoblotted for PINK1, the N-terminus of PARL, the C-terminus of PARL, and Hsp60. FIG. 9D includes two Western blots. Untransfected HeLa cells or HeLa cells stably expressing YFP-Parkin (HeLa/Parkin) were treated with DMSO or 2 μM CCCP without serum for 1 hr. Whole cell lysates (WCL) were run on SDS gels and immunoblotted for endogenous PINK1 and tubulin. FIG. 9E includes two Western blots. Parkin^{+/+} or Parkin^{-/-} MEFs transfected with PINK1-myc were treated with 2 μM CCCP in the absence of serum and fractionated. The mitochondria rich membrane fraction was immunoblotted for PINK1 and VDAC. Scale bars in all images=10 μm.

FIGS. 10A-10F show that Parkin recruitment to depolarized mitochondria requires PINK1 and its mitochondrial targeting N-terminus FIG. 10A includes fifteen fluorescent micrographs. Primary MEFs from PINK1^{+/+} or PINK1^{-/-} mice co-transfected with YFP-Parkin (green) and the indicated construct (vector, PINK1-V5, PINK1 kinase-deficient V5, or PINK1 156-581 [ΔN]-V5) in a 1:4 ratio were treated with DMSO or 20 μM CCCP in serum for 3 hours. Mitochondria were immunostained for Tom20 (red). FIG. 10B is a graph that quantitates co-localization between YFP-Parkin and mitochondria in FIG. 10A was scored for ≧100 cells/condition in ≧3 independent experiments. FIG. 10C is a graph that quantitates YFP Parkin accumulation on mitochondria. Transformed MEFs from independently generated PINK1^{+/+} and PINK1^{-/-} mice were transfected and treated as in FIG. 10A and were scored as in FIG. 10B. FIG. 10D includes six micrographs. M17 human neuroblastoma cells stably transduced with control shRNA or PINK1 shRNA were treated with 10 μM CCCP in serum for 3 hours and imaged as in FIG. 10A. FIG. 10E is a graph that quantitates co-localization between YFP-Parkin and mitochondria in FIG. 10D, which was scored as described in FIG. 10B. FIG. 10F includes two Western blots. Control shRNA and PINK1 shRNA M17 cells transfected and treated as in FIG. 10D were fractionated into mitochondria-rich membrane fraction (Memb) and supernatant (Sup). Fractions were run on SDS gels and immunoblotted with anti-Parkin and anti-VDAC antibodies. Loading was adjusted for approximately equal concentrations of YFP-Parkin in the post-nuclear supernatants (PNS) between the two cell types. Scale bars in all images=10 μm.

FIGS. 11A and 11B show that PINK1 is required for Parkin recruitment to mitochondria. FIG. 11A includes ten fluorescent micrographs showing SV40 transformed PINK1^{-/-} MEFs co-transfected with YFP-Parkin (green) and vector, PINK1, or PINK1 KD were treated with 20 μM CCCP for 3 hours. Cells were immunostained for Tom20 (red). FIG. 11B is two Western blots. M17 neuroblastoma cells stably transduced with control shRNA or PINK1 shRNA and transfected with YFP-Parkin were treated with DMSO or 10 μM CCCP for 3 hours and fractionated into post-nuclear supernatant (PNS), mitochondria-rich heavy membrane fraction (HMF), and supernatant (Sup). Fractions were run on SDS gels and immunoblotted for Parkin, PINK1, and VDAC. Scale bars in images=10 μm.

FIGS. 12A-12F show that PINK1 is required for Parkin-induced autophagy of depolarized mitochondria. FIG. 12A includes twelve fluorescent micrographs of primary MEFs from PINK1^{+/+} or PINK1^{-/-} mice co-transfected with YFP-Parkin were treated with DMSO or 20 μM CCCP in serum for 24 hours. Mitochondria were stained with an anti-Tom20 antibody. FIG. 12B is a graph showing the percent of cells with no detectable mitochondria in FIG. 12A, which was scored for >150 cells/condition in ≧3 independent experiments. FIG. 12C shows six micrographs of M17 human neuroblastoma cells stably transduced with control shRNA or PINK1 shRNA were treated with 10 μM CCCP for 24 hours and stained as in FIG. 12A. FIG. 12D is a graph showing the percent of cells with no mitochondria was scored for FIG. 12C as described in FIG. 12B. Scale bars in all images=10 μm. (E) M17 cells stably transduced with control shRNA or PINK1 shRNA were treated with DMSO or 10 μM CCCP for 24 hours and stained with Mitotracker Green (MTG). MTG, which stains mitochondrial lipid in a membrane potential independent manner, is a sensitive measure of mitochondrial mass. The graph represents change in Mitotracker Green intensity between DMSO and CCCP treated samples in three independent experiments. FIG. 12F is a graph that quantitates relative MTG fluorescence in M17 cells stably transduced with control shRNA or PINK1 shRNA were pulsed with Mitotracker Green in the presence of CCCP. Loss of MTG intensity was measured at 0 hours, 16 hours, and 24 hours with a plate reader. The graph shows data from three biological replicates and is representative of three independent experiments.

FIGS. 13A-13D show the kinetics of Parkin recruitment are modulated by PINK1 expression. FIG. 13A shows images of HeLa cells transfected with mCherry-Parkin (red) alone or mCherry-Parkin (red) and PINK1-YFP in a 1:1 ratio. The cells were imaged live following the addition of 10 μM CCCP in serum at time point 0 min FIG. 13B lists the vectors used for transfection of HeLa cells with mCherry-Parkin and the indicated construct in a 1:1 ratio were treated as in (A) and imaged live (1 frame/minute) following the addition of CCCP. Time to the beginning of Parkin translocation was defined as the first appearance of puncta in ≧2 quandrants of the cell for ≧2 consecutive images for ≧6 cells in ≧3 independent experiments. FIG. 13C shows six live confocal images of HeLa cells transfected with YFP-Parkin (green) or YFP-Parkin (green) and PINK1-myc (in a 1:4 ratio). Cells were loaded with TMRE (red) to stain polarized mitochondria. Cells were not treated with CCCP. Scale bars in last image=10 μm. FIG. 13D is a graph that quantitates results in cells treated as described in FIG. 13C, which were scored for co-localization between YFP-Parkin and TMRE. ≧50 cells/experiment were scored in ≧3 independent experiments.

FIGS. 14A-H show stable expression of PINK1 on the outer mitochondrial membrane is sufficient for Parkin recruitment. FIG. 14A is a schematic diagram depicting the construction of PINK1-YFP (green), PINK1 (111-581)-YFP (green), and OPA3-PINK1 (111-581)-YFP (green). FIG. 14B includes six confocal images depicting the localization of PINK1-YFP, PINK1 (111-581)-YFP, and OPA3-PINK1 (111-581)-YFP in HeLa cells. Mitochondria are stained with the potentiometric dye TMRE (red). FIG. 14C is three Western blots. HeLa cells were transfected with PINK1-YFP, PINK1 (111-581)-YFP, or Opa3-PINK1 (111-581)-YFP and treated with DMSO or 2 μM CCCP in serum free media for 3 hours. Whole cell lysates (WCL) were run on SDS gels and immunoblotted for PINK1, GFP, and tubulin. FIG. 14D includes three confocal images of HeLa cells co-transfected with mCherry-Parkin (red) and PINK1-YFP (green), PINK1 (111-581)-YFP (green), or OPA3-PINK1 (111-581)-YFP (green). Cells were not treated with CCCP. FIG. 14G includes six micrographs of HeLa cells in FIG. 14F, which were scored for mCherry-Parkin forming puncta characteristic of mitochondria in ≧150 cells in ≧3 independent experiments. Cells were not treated with CCCP. FIG. 14F is a graph that quantitates the results of AP21967 on Parkin on mitochondria. HeLa cells were transfected with FRB-PINK1 (111-581)-YFP, which is in the cytosol, TOM20(1-33)-FKBP, which is on mitochondria, and mCherry-Parkin. In the presence of the rapamycin analogue, AP21967, the FRB and FKBP domains of the respective fusion proteins (PINK1(111-581) and TOM20's outer mitochondrial membrane anchor) heterodimerize, if they have access to the same compartment (e.g., the cytosol). Cells treated with vehicle or 250 nM of AP21967 for 8 hours were scored for mCherry-Parkin in puncta characteristic of mitochondria in ≧150 cells in ≧3 independent experiments. FIG. 14G includes six confocal images of HeLa cells transfected with PINK1-YFP (green), PINK1 (111-581)-YFP (green), or OPA3-PINK1 (111-581)-YFP (green) with or without ECFP-Parkin and cultured for 96 hours in the absence of CCCP. Cells were immunostained for Tom20 (red). FIG. 14H is a graph that represents results of cells treated as in FIG. 14G, which were scored for the absence of detectable mitochondria in ≧150 cells in ≧3 independent experiments. Scale bars in all images=10 μm.

FIGS. 15A-D show increased expression of PINK1 on the outer mitochondrial membrane is sufficient for Parkin recruitment. FIG. 15A is a schematic diagram illustrating the construction of PINK1-YFP, FRB-PINK1 (111-581)-YFP, and Tom20(1-33)-FKBP. The FRB and FKBP domains heterodimerize in the presence of the rapamycin analogue AP21967 if they are in the same compartment. FIG. 15B includes five live images of HeLa cell were transfected with PINK1 (111-581)-YFP and Tom20 (1-33)-FKBP. 250 nM of the rapamycin analogue, AP21967, was added at time point 0. FIG. 15C includes four confocal images depicting the localization of FRB-PINK1 (111-581)-YFP (green) co-transfected with Tom20 (1-33)-FKBP following treatment with vehicle or 250 nM of AP21967 for 30 minutes. Mitochondria are labeled with the potentiometric dye TMRE (red). FIG. 15D includes four confocal images depicting the localization of FRB-PINK1 (111-581)-YFP (green) and mCherry-Parkin (red) following treatment with vehicle or 250 nM of AP21967 for 8 hours. Scale bars in all images=10 μm.

FIGS. 16A-16E show that PINK1 accumulation following depolarization with CCCP may be required for Parkin recruitment. FIG. 16A includes two Western blots. HeLa cells stably expressing YFP-Parkin were treated with 2 μM CCCP 1 hr alone or CCCP 1 hr+2 μM CHX (30 minutes pretreatment and 1 hr treatment) in the absence of serum. Whole cell lysates were run on SDS gels and immunoblotted for endogenous PINK1 and the mitochondrial protein VDAC. FIG. 16B includes two Western blots. Cells were treated as in FIG. 16A and were fractionated. The mitochondria-enriched membrane fraction was run on SDS gels and immunoblotted for endogenous PINK1 and VDAC. FIG. 16C includes six micrographs of HeLa cells transfected with YFP-Parkin (green) and treated with 10 μM CCCP 1 hr alone, CCCP+10 μM of actinomycin (30 minutes pretreatment and 1 hr treatment), or CCCP 1 hr+100 μM CHX (30 minutes pretreatment and 1 hr treatment) in the presence of serum and immunostained for Tom20 (red). FIG. 16D is a graph that quantitates co-localization between YFP-Parkin and mitochondria in FIG. 16F, which was scored for ≧150 cells/condition in ≧3 independent experiments. FIG. 16E includes two Western blots. HeLa cells stably expressing YFP-Parkin were treated as in FIG. 16A and fractionated. The mitochondria-rich fraction was run on an SDS gel and immunostained for Parkin. Scale bars in all images=10 μm.

FIGS. 17A-17C show that putative PINK1 phosphorylation sites on Parkin, T175 and T217, are not sufficient for Parkin recruitment. FIG. 17A shows an alignment of highly conserved Parkin unique region/domain containing T175 and T217. Arrows on top show positions of threonines 175 and 217 and the disease-causing mutation C212. Brackets on the bottom of the alignment point to the conserved cysteine and histidine residues forming putative zinc-binding sites I and II of the RING0 domain. FIG. 17B includes micrographs of HeLa cells that were transfected with YFP-Parkin (green) containing the indicated point mutations and treated with DMSO or CCCP for 1 hr. Mitochondria were labeled with anti-Tom20 antibody (red). (C) Co-localization between YFP-Parkin and mitochondria in FIG. 17A was scored for >150 cells/condition in ≧3 independent experiments. Scale bars in all images=10 μm.

FIGS. 18A-18C show that disease-causing PINK1 mutants fail to reconstitute Parkin recruitment to depolarized mitochondria. FIG. 18A includes eighteen micrographs showing primary MEFs from PINK1^{-/-} mice co-transfected with YFP-Parkin (green) and indicated V5-tagged constructs in a 1:4 ratio that were treated with DMSO or 20 μM CCCP in serum for 3 hours. Mitochondria were stained with an anti-Tom20 antibody (red). Scale bar in images=10 μm. FIG. 18B is a graph that quantitates the co-localization between YFP-Parkin and mitochondria in FIG. 18A, which was scored for >150 cells/condition in ≧3 independent experiments. FIG. 18C includes three Western blots. HeLa cells stably expressing YFP-Parkin were transfected with the indicated V5 tagged constructs, treated with DMSO or 2 μM CCCP for 3 hours in serum-free media, and fractionated. The mitochondria-rich membrane fraction was run on an SDS gel and immunoblotted for PINK1, the V5 tag, and the mitochondrial protein VDAC.

FIG. 19A is a Western blot, which shows that disease-causing mutations in PINK1 do not affect PINK1 induced accumulation. HeLa cells stably expressing Parkin transfected with the indicated V5 tagged constructs were treated with DMSO or 2 μM CCCP in serum-free media. Whole cell lysates were run on SDS gels and immunoblotted for PINK1, the V5 tag, and tubulin.

FIGS. 20A-20E show that disease-causing mutations in Parkin disrupt Parkin recruitment to mitochondria and/or Parkin-induced mitophagy. FIG. 20A is a schematic and micrographs of HeLa cells that were transfected with YFP-Parkin (white and green) containing indicated mutations and treated with CCCP for 1 hr. Mitochondria were labeled with an anti-Tom20 antibody (red). FIG. 20B is a graph that quantitates co-localization between YFP-Parkin and mitochondria in FIG. 20A scored for ≧150 cells/condition in ≧3 independent experiments. FIG. 20C includes four Western blots. HeLa cells were transfected and treated as in FIG. 20A and fractionated into post-nuclear supernatant (PNS), mitochondria-rich heavy membrane fraction (HMF), and supernatant (Sup). Fractions run on SDS gels and immunoblotted for Parkin and VDAC. FIGS. 20D and E includes results with HeLa cells transfected as in FIG. 20A and treated with CCCP or DMSO for 24 hours. FIG. 20D is a graph that shows the number of HeLa cells with no mitochondria scored for ≧150 cells/condition in ≧3 independent experiments. FIG. 20E provides six images of WT, R42P, and R275W Parkin (green) stained as in FIG. 20A. * indicates engineered mutation; all others have been linked to Parkinson's disease. Scale bars in all images=10 μm.

FIGS. 21A-21F show that mutations in Parkin's Ubiquitin-like Domain (UBL) partially disrupt Parkin recruitment to mitochondria. FIG. 21A provides an alignment of part of the UBL amino acid sequences from orthologous Parkin proteins. * indicates position of patient mutations (R42P and R46) and engineered mutation (I44A) examined below. Red box indicates position of beta-pleated sheet containing I44, a key residue for interactions between UBL domains and Ubiquitin-Binding Domains. FIG. 21B shows the structure of UBL (PDB 1IYF) with position of patient mutations (R42P and A46P) highlighted in blue and position of engineered mutation I44A highlighted in red. FIGS. 21C and D are micrographs showing HeLa cells transfected with YFP-Parkin containing the indicated mutations and treated with CCCP for 1 hr (C) or 24 hours (D). Mitochondria labeled with Tom20 antibody. FIG. 21E is a graph that quantitates co-localization between YFP-Parkin and mitochondria in FIG. 21C scored for ≧150 cells/condition in ≧3 independent experiments. FIG. 21F is a graph that shows the percentage of cells with no mitochondria transfected and treated as described in (D) scored for ≧150 cells/condition in ≧3 independent experiments. Scale bars in all images=10 μm.

FIGS. 22A-22E show that mutations in Parkin disrupt Parkin recruitment and/or Parkin-mediated mitophagy. FIG. 22A is a graph showing the percentage of cells with Parkin on mitochondria. HeLa cells were transfected with YFP-Parkin (green) containing the indicated missense mutations were treated with 10 μM CCCP in serum for 24 hours were scored for Parkin co-localizing with mitochondria in ≧150 cells/condition in ≧3 independent experiments. Mitochondria were immunostained for Tom20 (red). FIG. 22B includes nine confocal images representing FIG. 22A. FIG. 22C includes six live images of HeLa cells transfected with YFP-Parkin R275W (green). 10 μM CCCP was added at time point 0. FIG. 22D is a graph showing the percentage of cells with Parkin aggregates. HeLa cells transfected with the indicated constructs were treated with DMSO or 10 μM CCCP for 24 hours and scored for the percentage of cells with visible aggregates in ≧150 cells/condition in ≧3 independent experiments. FIG. 22E includes eight micrographs of HeLa cells transfected with YFP-Parkin WT (green) or YFP-Parkin R275W (green) were treated with DMSO or CCCP for 1 hr and imaged live. Mitochondria were stained with the potentiometric dye TMRE (red). Scale bars in all images=10 μm.

FIG. 23 is a model depicting regulation of PINK1 stability on healthy and dysfunctional mitochondria by membrane potential. On healthy mitochondria PINK1 is constitutively imported, proteolytically cleaved into a cytosolic form, and degraded by the proteasome, resulting in low levels of mitochondrial PINK1. On damaged mitochondria with low membrane potentials (ΔΨ), however, PINK1 cleavage is blocked, leading to accumulation of mitochondrial PINK1 on the dysfunctional mitochondria. Accumulated PINK1 recruits Parkin to damaged mitochondria, which Parkin marks, likely by ubiquitination, for autophagic degradation.

FIGS. 24A-F show that YFP-Parkin accumulates on mitochondria following loss of mtDNA integrity and promotes the selective elimination of mutant mtDNA in COXICA65 cybrid cells. FIG. 24A includes fluorescent micrographs of HeLa cells transfected with Flag-tagged wt Twinkle and Flag-tagged G575D mutant Twinkle for 5 days were immunostained for Tom20 (mitochondria, red) and Flag (Twinkle, white; blue in merged image). FIG. 24B includes six micrographs of the parental 143B cell with 100% wild-type mtDNA and COXICA65 cybrid cell with ˜75% mutant mtDNA (G->A transition at 6930 nt in cytochrome c oxidase subunit I) that were transfected with YFP-Parkin (green), fixed and immunostained for Tom20 (mitochondria, red). FIG. 24C includes micrographs of 143B and COXICA65 co-expressing YFP-Parkin (green), vMIA-myc and mito-CFP (white; blue in the merged images) were stained with 2.5 nM of TMRE (red) for 1 hour. FIG. 24D is a graph that quantitates cells scored for YFP-Parkin on mitochondria in the presence or absence of vMIA-myc. More than 60 cells were counted in each sample. FIG. 24E includes a schematic of vectors used for transfection. COXICA65 cybrid cells were transfected with YFP-Parkin (Parkin), YFP vector (vector), or left untransfected (N/A). The transfected cells were enriched with YFP signal by FACS following transfection for 45 days (Parkin 45 days) and 60 days (Parkin 60 days). The parental 143B cell carries 100% wild-type mtDNA. The ratio of wild-type and mutant mtDNA was analysis by PCR-RFLP. A 217-bp was amplified from both wild-type and mutant mitochondria DNA respectively. Following AluI digestion, the wild-type mtDNA (which possess one AluI site) showed 125 and 92-bp fragments and mutant mtDNA (which possess two AluI sites) showed 125, 63 and 29-bp fragment. The 29-bp fragment was dim by EtBr staining and not shown here. FIG. 24F is a graph that shows results of a Cytochrome c oxidase activity (COX) assay. The COX activity for each sample is reported relative to 143B (=100%), which contains 100% wild-type mtDNA. Scale bar=10 μm and 2 μm in the magnified images.

FIGS. 25A and 25B show quantitative FACS analysis of TMRE intensity. FIG. 25A includes graphs showing a FACS analysis 143B, Rho0, COXICA65 and Cytb3.0 were stained with TMRE as described in materials and methods. 10,000 cells were analyzed for each sample. The mean and standard deviation were calculated from two experiments. These results are quantitated in FIG. 25B showing that the COXICA65 cells have mitochondria with lower membrane potential than the control 143B cells.

FIGS. 26A-26F show mutant mtDNA reaccumulation. COXICA65 cybrid cells were transfected with YFP-Parkin (Parkin), YFP vector (vector), or left untransfected (N/A). In two independent experiments, cells were sorted by YFP signal over the course of 180 and 200 days, respectively. In the first experiment a relatively moderate level of YFP-Parkin expression (Parkin M) was achieved, while in the second experiment, a relatively high level of YFP-Parkin expression (Parkin H) was achieved. FIGS. 26A-26C are gels. FIG. 26A is a gel showing results with wt and mutant mtDNA analyzed by PCR-RFLP. 143B and COXICA65 expressing YFP-Parkin (Parkin M and Parkin H) or YFP vector (vector) were analyzed FIG. 26A were continually cultured for 40 days FIGS. 26B and 67 days FIG. 26C in the absence of FACS selection. FIG. 26D includes six micrographs of COXICA65 cells Parkin-enriched for wild-type mtDNA (Parkin H, 67 days post-enrichment) were fixed and stained with Tom20 antibody (mitochondria, blue) and COXI antibody (red). YFP-Parkin is green. Scale bar=20 μm. FIG. 26AE is a graph showing the percentage of COXI positive and negative cells were scored for the Parkin enriched COXICA65 (Parkin H 67 days post-enrichment) (the upper panel a), untransfected 143B, and COXICA65 cell lines. More than 600 cells lacking detectable YFP-Parkin signal were counted in each sample. FIG. 26F is a graph showing results of a Cytochrome c oxidase activity (COX) assay. COX activity for each sample is reported relative to 143B, which contains 100% wild-type DNA.

FIGS. 27A and 27B show results of ³²P-labeled PCR-RFLP. The samples analyzed in FIGS. 26A and 26C were labeled with [γ³²P] dCTP at the last cycle of PCR. FIG. 27A shows PCR products run on 10% polyacrylamide gels. ³²P radiation was detected using the PhosphoImage system. FIG. 27B is a graph. The intensity of each band in 27a was quantified and normalized to intensity of 125-bp fragment for each sample. The percentage of wt mtDNA was calculated by dividing the 92-bp fragment intensity by the sum of the 92-bp, 63-bp and 29-bp fragment intensities.

FIG. 28 includes six fluorescent micrographs showing COXI in 143B, Rho0 and COXICA65 cybrid. Cells were fixed and immunostained for rabbit Tom20 antibody (blue) and mouse COXI (red). All images were scanned using the same confocal settings as were used in FIG. 2d. Scale bar=20 μm.

FIGS. 29A-29G shows that constitutive cleavage of PINK1 is mediated by PARL. FIG. 29A is an immunoblot of HeLa cells transfected with scrambled control siRNA or PARL siRNA. After 4 hrs incubation with or without 10 μM CCCP, mitochondria were isolated and mitochondrial protein extracts were assayed for endogenous levels of PINK1 and PARL by immunoblotting. VDAC1 is a mitochondrial marker. FIG. 29B is an immunoblot of MEFs from PARL WT and KO mouse transfected with PINK1-V5/His for 2 days and treated with DMSO or CCCP (10 μM) for 4 hrs. Exogenous PINK1 levels were assayed by immunoblotting. Tubulin is a loading control. FIG. 29C is an immunoblot of PARL WT and KO MEFs transfected with PINK1-V5/His as in FIG. 29B and treated with DMSO or MG132 (10 μM). After 4 hrs of treatment, cells were fractionated and then exogenous PINK1 level in mitochondrial fraction were measured with immunoblotting. VDAC1, mitochondrial loading control. Red arrow, 52 kDa PINK1, hereafter. FIG. 29D shows results from ³⁵5-Met labeled PINK1 incubated for different times with mitochondria isolated from PARL WT or KO MEFs in the presence or absence of 1 μM CCCP. Following import, samples were treated with or without 5 μg/ml Proteinase K (PK). Radiolabeled PINK1 was detected using digital autoradiography. Stars, non-specific bands. FIG. 29E shows that ³⁵S-PINK1 was imported into PARL KO mitochondria for 60 min as in FIG. 29D and these mitochondria were incubated in the presence or absence of high PK (100 μg/ml) for 10 min Hsp70, Htra2/Omi and Tom20 were identified by immunoblotting as markers for mitochondrial matrix, inter membrane space and outer membrane, respectively. FIG. 29F is a photomicrograph of HeLa cells stably expressing YFP-Parkin and transfected with control (siCtrl) or PARL siRNA (siPARL) for 192 hrs. Following transfection, cells were treated with either DMSO or CCCP (10 μM) for 1 hr, stained with TMRE and analyzed by live-cell imaging. Scale bar=20 μm. FIG. 29G is a graph of results from PARL WT and KO MEFs transfected with PINK1-YFP and mCherry-Parkin were treated with DMSO or CCCP (10 μM) for 3 hrs. Cells (≧50 per treatment) were counted for mitochondrial translocation of YFP-Parkin. Counting results were represented as mean±standard error from 4 replicates.

FIGS. 30A-30C shows that the 52 kDa form of endogenous PINK1 is found inside mitochondria and does not recruit Parkin. FIG. 30A is an immunoblot of HeLa cells initially treated with MG132 (50 μM) for 10 hrs and then together with CCCP (10 μM) for a final 3 hrs. Cells were then fractionated and analyzed by immunoblotting using antibodies against the indicated proteins. FIG. 30B is an immunoblot of PINK1, cytochrome c (Cyt. C) and Tim23 The mitochondrial fraction from FIG. 30A was subjected to alkaline extraction using sodium carbonate, and immunoblotted for PINK1, cytochrome c (Cyt. C) and Tim23 FIG. 30C is an immunoblot of PINK1, Tom20, Cyt. c, AIF, and Hsp70. Mitochondria from FIG. 30A were incubated for 30 min on ice with various concentrations of PK followed by immunoblotting using antibodies against PINK1 and the indicated mitochondrial markers. FIG. 30D is a set of photomicrographs of HeLa cells stably expressing YFP-Parkin and treated with either DMSO, CCCP (10 μM), or MG132 (10 μM) for 3 hrs followed by staining with TMRE and confocal imaging. Scale bar=20 μm.

FIG. 31A-31D show that point mutations in the transmembrane domain of PINK1 partially inhibit its proteolytic cleavage. FIG. 31A shows the amino acids throughout the predicted transmembrane domain of PINK1 were mutated to phenylalanine (aa 91-98) or tryptophane (aa 99-110). FIG. 31B show HeLa cells transfected with the indicated PINK1-YFP mutants treated with either DMSO, CCCP (10 μM) or MG132 (10 μM) for 3 hrs. Cells lysates (20 μg) were subjected to SDS-PAGE and immunoblotting using antibodies against PINK1 and tubulin. Green arrow, FL and ΔMTS-PINK1; red arrow, 52 kDa PINK1 FIG. 31C shows the band intensity of FL PINK1 in DMSO or CCCP-treated lanes from FIG. 31B was densitometrically measured using Multi Gauge (Fujifilm). Following corrections for background and loading, the band intensity ratio of DMSO/CCCP-treated sample for each PINK1 mutant was measured. FIG. 31D are a panel of photomicrographs of YFP tagged WT PINK1 and PINK1 R98F mutants transfected into HeLa cells. Cells were stained with Mitotracker Red prior to treatment with CCCP (10 μM) for 3 hrs and analyzed by confocal microscopy. Bottom panels display enlarged views of the white boxed areas. Scale bar=20 μm.

FIG. 32A to 32F shows the PINK1 R98F mutant resistant to PARL-mediated cleavage is located inside mitochondria. FIG. 32A is an immunoblot of extracts of Mitochondria isolated from HeLa cells transfected with YFP-tagged PINK1 R98F were incubated with various concentration of PK for 30 min on ice, and immunoblotted for PINK1, Tom20, Cyt. c, AIF, Hsp70. Green arrow, FL and ΔMTS-PINK1; red arrow, 52 kDa PINK1. FIG. 32B is a panel of photomicrographs of HeLa cells transfected with mito-YFP and treated with CCCP (10 μM) for 3 hrs, followed by incubation in either PBS alone or PBS containing 0.005% digitonin or 0.25% Trixon X-100 (TX-100). Cells were immunostained using antibodies against Tom20 and Cyt. c and analyzed by confocal microscopy. Scale bar=20 μm. FIG. 32C is a panel of photomicrographs of HeLa cells transfected with YFP-tagged WT PINK1 or PINK1 R98F mutant for 18 hrs. Cells were then treated with CCCP for 3 hrs, permeabilized and immunostained with indicated antibodies. Images were taken by confocal microscopy. Scale bar=20 μm. FIG. 32D is a graph showing HeLa Cells (≧150/condition) stained in FIG. 32C counted for GFP immunofluorescence. Counting results were represented as mean±standard error from 4 replicates. FIG. 32E is a panel of photomicrographs of HeLa cells co-transfected with YFP-tagged PINK1 R98F mutant and mCherry-Parkin and incubated with either DMSO or CCCP (10 μM) for 1 hr followed by confocal imaging. Scale bar=20 μm. FIG. 32F is a graph of PINK1 KO MEFs transfected with YFP-tagged WT PINK1 or PINK1 R98F mutant and treated with DMSO or CCCP (10 μM) for 3 hrs and cells and counted for mitochondrial translocation of Parkin (≧50 cell counts for each sample). Counting results were represented as mean±standard error from 4 replicates.

FIG. 33 is a graphical presentation of a model of PINK1 import and processing.

FIGS. 34A-34C show that transmembrane domain deleted-PINK1 fails to recruit Parkin following mitochondrial depolarization. FIG. 34A is an immunoblot of HeLa cells transfected with WT or Δ[91-117] (ΔTM)-PINK1-YFP for 18 hrs and treated with 10 μM CCCP for different times as indicated. Cells were fractionated and the mitochondrial fractions were immunoblotted for PINK1. Tom20 was used as a mitochondrial marker. FIG. 34B is a graph plotting the band intensity in each lane in FIG. 34A densitometrically measured using Multi Gauge (Fujifilm). After correction for background, PINK1 band intensity in each lane was normalized to the loading control (VDAC) and calculated for fold increase. FIG. 34C is a panel of photomicrographs of PINK1 KO MEFs transfected with mCherry-Parkin and either WT or Δ91-117-PINK1-YFP. Following treatment with DMSO or CCCP (10 μM) for 3 h, Parkin translocation was examined using Confocal microscopy. White bar; 20 μm.

FIGS. 35A and 35B show the protein sequence alignment of the predicted transmembrane domain of PINK1 from various species. FIG. 35A is the amino acid sequences of the predicted transmembrane domain of PINK1 from indicated species aligned using the ClustalW algorithm (http://www.uniprot.org/). The putative transmembrane domains are indicated with a red box. ‘*’, fully conserved; ‘:’, strongly conserved; ‘.’, weakly conserved residue. FIG. 35B are hydropathy plots for identifying the putative transmembrane regions were created by the program DAS (Density Alignment Surface; Cserzo et al., 1997). Sequences of full-length PINK1 proteins (Human and Drosophila) were used for the analyses.

FIGS. 36A and 36B show that the R98F PINK1-YFP mutant accumulates in mitochondria without mitochondrial uncoupling but does not recruit Parkin. FIG. 36A is an immunoblot of WT or R98F mutant PINK1-YFP transfected into HeLa cells and incubated with DMSO or CCCP (10 μM) for 3 hrs. Cells were fractionated to mitochondria enriched and cytosolic fractions. Whole cell lysates, mitochondrial, and cytosolic fractions were analyzed for the level of expressed PINK1 with immunoblotting. As shown in the middle top panel, a fraction of the 52 kDa form of ectopic PINK1 was found in the cytosolic fraction and might be the artifact of overexpression (See FIG. 2a for endogenous 52 kDa PINK1). Green arrow, FL and ΔMTS-PINK1; red arrow, 52 kDa PINK1. β-actin and Tim23 are loading controls. FIG. 36B is a graph of HeLa cells transfected with WT or R98F mutant PINK1-YFP together with mCherry-Parkin. After 1 hr incubation with DMSO or CCCP (10 μM), cells (≧150/condition) were counted for mitochondrial translocation of Parkin. Counting results were represented as mean±standard error from 4 replicates.

### DETAILED DESCRIPTION OF THE INVENTION

The invention provides compositions and methods for the treatment of diseases associated with a mitochondrial defect (e.g., a mutation in mitochondrial DNA) or a reduction in mitochondrial function.

Mitochondrial dysfunction causes severe syndromes, such as MELAS, MILS and LHON. Mitochondrial genomes with deleterious mutations replicate in cells along with wild-type genomes resulting in a state of heteroplasmy. Loss of function mutations in PINK1 and Parkin cause parkinsonism in humans and mitochondrial dysfunction in model organisms. Parkin is selectively recruited from the cytosol to damaged mitochondria to trigger their autophagy.

The invention is based, at least in part on the following discoveries; Parkin was selectively recruited to dysfunctional mitochondria with low membrane potential in mammalian cells. After recruitment, Parkin mediates the engulfment of mitochondria by autophagosomes and the selective elimination of impaired mitochondria. These results show that Parkin promotes autophagy of damaged mitochondria and implicates a failure to eliminate dysfunctional mitochondria in the pathogenesis of Parkinson's disease. Moreover, Parkin recognition of PINK1 accumulation on mitochondria was found to be both necessary and sufficient for Parkin recruitment to mitochondria. Expression of PINK1 on individual mitochondria was regulated by voltage-dependent proteolysis to maintain low levels of PINK1 on healthy, polarized mitochondria, while facilitating the rapid accumulation of PINK1 on mitochondria that sustain damage. Disease causing mutations in PINK1 and Parkin disrupt Parkin recruitment and Parkin-induced mitophagy at distinct steps. These findings indicate that PINK1 signals mitochondrial dysfunction to Parkin and Parkin promotes their elimination. The cytosolic E3 ligase, Parkin, translocates to dysfunctional mitochondria and induces their autophagic elimination. As reported herein, overexpression of Parkin can selectively eliminate mitochondria with deleterious COXI mutations in heteroplasmic cells, enriching cells for wild-type mtDNA and restoring cytochrome c oxidase activity. These data support the model that Parkin functions in a mitochondrial quality control pathway. Additionally, they suggest that increasing levels of Parkin expression might ameliorate certain mitochondrial diseases. Accordingly, the invention provides compositions and methods for increasing levels of Parkin and/or Pink1 for the treatment of diseases associated with mitochondrial dysfunction.

In other embodiments, the invention is based, at least in part, on the discovery that the mitochondrial inner membrane rhomboid protease PARL mediates cleavage of PINK1 dependent on mitochondrial membrane potential. In the absence of PARL, the constitutive degradation of PINK1 is inhibited, stabilizing a 60 kDa form inside mitochondria. When mitochondrial membrane potential is dissipated PINK1 accumulates as a 63 kDa full-length form on the outer mitochondrial membrane where it can recruit Parkin to impaired mitochondria. Thus, differential localization to the inner and outer mitochondrial membranes appears to regulate PINK1 stability and function. Accordingly, the invention features compositions and methods for reducing PARL activity, thereby increasing PINK1 accumulation.

Diseases Associated with Mitochondrial Dysfunction

One in 4,000 children in the United States will develop mitochondrial disease by the age of 10 years. One thousand to 4,000 children per year in the United Sates are born with a type of mitochondrial disease. In adults, many diseases of aging have been found to be associated with defects of mitochondrial function. These include, but are not limited to, type 2 diabetes, Parkinson's disease, atherosclerotic heart disease, stroke, Alzheimer's disease, and cancer. In addition, many medicines can injure the mitochondria.

Most of our body's nucleated cells contain 500 to 2000 mitochondria. In the cone cell photoreceptors of the eye, mitochondria make up 80% of the intracellular volume. In extraocular muscles like the lateral rectus, they account for 60%, and in heart muscle they comprise 40% of the volume of the cell. Mitochondria are the only cellular organelles in animals known to have their own DNA* (mitochondrial DNA* or mtDNA), distinct from the nuclear DNA* (nDNA). Defects in nDNA can be inherited from either parent but defects in the genes of the mtDNA are maternally inherited. A typical cell contains thousands of copies of mtDNA, and an electrochemically discrete mitochondrion may contain zero to hundreds of copies of the mitochondrial genome depending on the interconnectivity of the mitochondrial network. Within the cells of a patient affected with a mitochondrial disease, mutated mtDNA typically coexists with wild-type mtDNA. In this heteroplasmic state, wild-type and mutant mtDNA are packed in separate nucleoids and rarely mix even though nucleoids move relatively freely in mitochondria. The severity of cellular dysfunction and disease caused by a given mtDNA mutation depends on the ratio of mutant mtDNA to wild-type mtDNA in the cell. Experimentally shifting a population of mtDNA away from the mutant DNA toward wild-type mtDNA improves mitochondrial function within the cell and tissue, and represents a promising therapeutic strategy for diseases in which mtDNA mutations contribute to the pathogenesis.

Mitochondrial DNA (mtDNA) mutations are responsible for a number of severe syndromes, with symptoms ranging from epilepsy and encephalopathy to lactic acidosis and diabetes. Some disorders known to be associated with mtDNA mutations include, but are not limited to, NARP—neurogenic muscular weakness, ataxia, retinitis pigmentosa, MSS—multiple sclerosis-like syndrome; MCIM—maternally inherited cardiomyopathy; PEO—progressive external ophthalmoplegia; MERRF—myoclonic epilepsy with ragged-red fibers; Myoneurogastrointestinal disorder and encephalopathy (MNGIE), Pearson Marrow syndrome, Kearns-Sayre-CPEO, Leber hereditary optic neuropathy (LHON), Aminoglycoside-associated deafness, Diabetes with deafness, Luft disease, Leigh syndrome (Complex I, COX, PDH), Alpers Disease, MCAD, SCAD, SCHAD, VLCAD, LCHAD, Glutaric aciduria II, and Lethal infantile cardiomyopathy. In addition, somatically acquired mtDNA mutations have been linked to the pathogenesis of common diseases, such as cancer, diabetes mellitus, and neurodegenerative disorders. For example, patients with sporadic Parkinson's disease have a greater number of functionally deleterious mtDNA mutations in their substantia nigral neurons compared to age matched controls, and increased mtDNA deletions, as is observed in patients with multiple mtDNA deletion syndromes, appears to be sufficient to cause parkinsonism.

### Parkinson's Disease

Parkinson's disease is a common neurodegenerative disorder with no disease-modifying therapy presently available for its treatment. Study of recessive forms of familial Parkinson's disease, such as those resulting from mutations in the E3 ubiquitin ligase Parkin or the mitochondrial kinase PINK1, may reveal disease mechanisms important to the development of disease in these families as well as those suffering from sporadic Parkinson's disease.

Although the cause of sporadic Parkinson's disease is likely complex, several lines of evidence link mitochondrial dysfunction to its pathogenesis. Mitochondria within the substantia nigra pars compacta (SNpc), a midbrain region that is preferentially affected in Parkinson's disease, have a higher somatic mitochondrial DNA (mtDNA) mutation rate than all other regions of the brain examined. Increased mitochondrial damage in the SNpc, particularly to mtDNA, has been associated with sporadic Parkinson's disease and mitochondrial dysfunction is sufficient to cause parkinsonism in patients with rare multiple mtDNA deletion syndromes and in animal models with decreased mtDNA expression. In addition, toxins such as MPTP and rotenone, which are believed to increase reactive oxygen species from complex I of the electron transport chain, can induce a parkinsonian syndrome in humans and animal models. Since neurons in the SNpc are post-mitotic, any mitochondrial damage they acquire could accumulate over an organism's lifetime, leading to progressive mitochondrial dysfunction—including increased oxidative stress, decreased calcium buffering capacity, loss of ATP, and, eventually, cell death—unless quality control processes eliminate the damaged mitochondria.

Recent studies have linked Parkin and PINK1 in a pathway critical for the maintenance of mitochondrial integrity and function. Loss of either protein in Drosophila results in a similar phenotype, with mitochondrial damage preceding muscle degeneration, as well as disrupted spermatogenesis and death of dopaminergic neurons. Interestingly, overexpression of Parkin can partially compensate for PINK1 loss, but PINK1 overexpression cannot compensate for Parkin loss, suggesting that PINK1 functions upstream of Parkin in a common pathway. Additionally, mice null for either Parkin or PINK1 exhibit increased oxidative damage and decreased mitochondrial function in the striatium (which receives projections from dopaminergic neurons); and primary cells from patients with loss of function mutations in Parkin or PINK1 have similar abnormalities.

As reported herein below, Parkin is selectively recruited to dysfunctional mitochondria with low membrane potential and, subsequently, promotes their autophagic degradation. This suggests that Parkin may limit mitochondrial damage by acting in a pathway that identifies and eliminates damaged mitochondria from the mitochondrial network. Full length PINK1 accumulates selectively on dysfunctional mitochondria. Parkin recruitment to depolarized mitochondria and subsequent Parkin-induced mitophagy are strictly dependent on PINK1's mitochondrial targeting signal and depolarization-induced accumulation. Without wishing to be bound by theory, these results strongly support a novel model for signaling between PINK1 and Parkin in response to mitochondrial damage. In this model, mitochondrial PINK1 is rapidly turned over on bioenergetically well-coupled mitochondria by proteolysis, but is selectively stabilized on mitochondria with low membrane potential. Selective accumulation of PINK1 on the impaired mitochondria recruits Parkin, and Parkin, in turn, induces the degradation of the damaged mitochondria. PINK1 and Parkin form a pathway for sensing and selectively eliminating damaged mitochondria from the mitochondrial network. Disease-causing mutations in PINK1 and/or Parkin disrupt this pathway at distinct steps, consistent with the pathway's importance for preventing early onset parkinsonism. Accordingly, compositions and methods for increasing PINK1 and Parkin expression or biological activity are useful for the treatment of diseases associated with mitochondrial dysfunction. In particular, the invention provides expression vectors that encode PINK1 and/or Parkin for expression in one or more tissues affected by mitochondrial dysfunction.

### Parkin and Pink1 Polypeptides and Analogs

The invention provides for the use of expression vectors encoding Parkin and/or Pink1 polypeptides. In one embodiment, the invention provides methods for optimizing a Parkin and/or Pink1 amino acid sequence or nucleic acid sequence by producing an alteration in the sequence. Such alterations may include certain mutations, deletions, insertions, or post-translational modifications. In other embodiments, the invention further includes analogs of any naturally occurring polypeptide of the invention. Analogs can differ from a naturally occurring polypeptide of the invention by amino acid sequence differences, by post-translational modifications, or by both. Analogs of the invention will generally exhibit at least 85%, more preferably 90%, and most preferably 95% or even 99% identity with all or part of a naturally occurring amino, acid sequence of the invention. The length of sequence comparison is at least 5, 10, 15 or 20 amino acid residues, preferably at least 25, 50, or 75 amino acid residues, and more preferably more than 100 amino acid residues.

Analogs can differ from the naturally occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

Amino acids include naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine. An amino acid analog is a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain); the term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a β-amino acid, or an N-methyl amino acid.

Amino acids and analogs are well known in the art Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. In addition to full-length polypeptides, the invention also includes fragments of any one of the polypeptides of the invention. Non-protein Parkin and/or Pink1 analogs having a chemical structure designed to mimic Parkin and/or Pink1 functional activity can be administered according to methods of the invention. Parkin and/or Pink1 analogs may exceed the physiological activity of the original polypeptide. Methods of analog design are well known in the art, and synthesis of analogs can be carried out according to such methods by modifying the chemical structures such that the resultant analogs exhibit the activity of a reference Parkin and/or Pink1 polypeptide. These chemical modifications include, but are not limited to, substituting alternative R groups and varying the degree of saturation at specific carbon atoms of a reference polypeptide. Preferably, the polypeptide analogs are relatively resistant to in vivo degradation, resulting in a more prolonged therapeutic effect upon administration. Assays for measuring functional activity include, but are not limited to, those described in the Examples below.

Also useful in the methods of the invention are Parkin and/or Pink1 polypeptides containing a detectable moiety. A “detectable moiety” is a composition that when linked with the nucleic acid or protein molecule of interest renders the latter detectable, via any means, including spectroscopic, photochemical (e.g., luciferase, GFP), biochemical, immunochemical, or chemical means. For example, useful labels include radioactive isotopes, magnetic beads, metallic beads, colloidal particles, fluorescent dyes, electron-dense reagents, enzymes (e.g., horseradish peroxidase, alkaline phosphatase), biotin, digoxigenin, or haptens. Such polypeptides can be used for the identification or imaging of a defective mitochondria

### Polynucleotide Therapy

Accordingly, polynucleotide therapy featuring a polynucleotide encoding a Parkin or Pink1 protein, variant, or fragment thereof is one therapeutic approach for treating a mitochondrial disease. Expression of such proteins in a cell comprising defective mitochondria is expected to promote the selective elimination of those defective mitochondria. Such nucleic acid molecules can be delivered to cells of a subject having a mitochondrial disease. The nucleic acid molecules must be delivered to the cells of a subject in a form in which they can be taken up so that therapeutically effective levels of a Parkin or Pink1 protein or fragment thereof can be produced.

Expression vectors encoding Parkin or PINK1 may be administered for global expression or may be used for the transduction of selected tissues. Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a Parkin or Pink1 protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a Parkin or Pink1 polynucleotide systemically.

Non-viral approaches can also be employed for the introduction of therapeutic to a cell of a patient requiring inhibition of a mitochondrial disease. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant a Parkin or Pink1 protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Inhibitory Nucleic Acids

Inhibitory nucleic acid molecules are those oligonucleotides that inhibit the expression or activity of a Parl polypeptide for the treatment of a mitochondrial disease. Such oligonucleotides include single and double stranded nucleic acid molecules (e.g., DNA, RNA, and analogs thereof) that bind a nucleic acid molecule that encodes a Parl polypeptide (e.g., antisense molecules, siRNA, shRNA) as well as nucleic acid molecules that bind directly to a Parl polypeptide to modulate its biological activity (e.g., aptamers).

Ribozymes

Catalytic RNA molecules or ribozymes that include an antisense PARL sequence of the present invention can be used to inhibit expression of a PARL nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of an Parl gene can be used to design small interfering RNAs (siRNAs). The 21 to 25 nucleotide siRNAs may be used, for example, as therapeutics to treat a mitochondrial disease or disorder.

The inhibitory nucleic acid molecules of the present invention may be employed as double-stranded RNAs for RNA interference (RNAi)-mediated knock-down of Parl expression. In one embodiment, Parl expression is reduced in an endothelial cell or an astrocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

Screens for Agents that Increase the Mitophagy of Defective Mitochondria

As reported herein below, PINK1 signals mitochondrial dysfunction to Parkin and Parkin promotes the selective elimination of those defective mitochondria. Given that subjects having mitochondrial defects have a mixed population of healthy and defective mitochondria, agents that selectively reduce the number of defective mitochondria are useful for the treatment of mitochondrial diseases. If desired, agents that increase the expression or biological activity of Parkin and/or Pink1 are tested for efficacy in enhancing the selective elimination of defective mitochondria in a cell (e.g., a cell comprising a genetic defect in mtDNA, a cell comprising a genetic mutation in Pink1, Parkin, a cell of the substantia nigra or a dopaminergic neuronal cell). Such methods are particularly useful for personalized medicine applications, for example, in identifying agents that are likely to be beneficial for a subject having a mitochondrial disease. In one example, a candidate compound is added to the culture medium of cells (e.g., neuronal cultures) prior to, concurrent with, or following the addition of a mitochondrial uncoupling agent or other agent that induces mitochondrial dysfunction. The number of defective mitochondria in the cells is then measured using standard methods. The number of defective mitochondria in the presence of the candidate agent is compared to the level measured in a corresponding control culture that did not receive the candidate agent. Alternatively, the agent's ability to promote the selective elimination of defective mitochondria is assayed in a cell comprising a defect in mtDNA. A compound that promotes an increase in Pink1 or Parkin expression or biological activity, or a reduction in defective mitochondria is identified as useful in the invention; such a candidate compound may be used, for example, as a therapeutic to prevent, delay, ameliorate, stabilize, or treat a disease or disorder characterized by mitochondrial dysfunction.

An agent isolated by this method (or any other appropriate method) may, if desired, be further purified (e.g., by high performance liquid chromatography). In addition, such candidate agents may be tested for their ability to modulate mitophagy in a cell comprising a mutation in mtDNA or in a neuronal cell. In other embodiments, the agent's activity is measured by identifying an increase in mitochondrial function, a reduction in cell death, or an increase in cell survival. Agents isolated by this approach may be used, for example, as therapeutics to treat a disease associated with mitochondrial dysfunction in a subject.

One skilled in the art appreciates that the effects of a candidate compound on a cell comprising defective mitochondria is typically compared to a corresponding control cell in the absence of the candidate compound.

Candidate agents include organic molecules, peptides, peptide mimetics, polypeptides, and nucleic acid molecules. Each of the sequences listed herein may also be used in the discovery and development of a therapeutic compound for the treatment of a mitochondrial disease. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra). Small molecules of the invention preferably have a molecular weight below 2,000 daltons, more preferably between 300 and 1,000 daltons, and most preferably between 400 and 700 daltons. It is preferred that these small molecules are organic molecules.

The invention also includes novel agents identified by the above-described screening assays. Optionally, such agents are characterized in one or more appropriate animal models to determine the efficacy of the compound for the treatment of a mitochondrial disease. Desirably, characterization in an animal model can also be used to determine the toxicity, side effects, or mechanism of action of treatment with such a compound. Furthermore, a novel agent identified in any of the above-described screening assays may be used for the treatment of a mitochondrial disease in a subject. Such agents are useful alone or in combination with other conventional therapies known in the art.

### Cells for Use in Screens

In one embodiment, the screens described herein are carried out in cybrid cells comprising mixed populations of wild-type and defective mitochondria. In another embodiment, the screens are carried out in cells comprising a defect in mtDNA.

In another embodiment, the screens described herein are carried out in dopaminergic cells having neuronal characteristics. Such cells are known in the art and include, for example, BE(2)-M17 neuroblastoma cells (Martin et al., J Neurochem. 2003 November; 87(3):620-30), Cath.a-differentiated (CAD) cells (Arboleda et al., J Mol Neurosci. 2005; 27(1):65-78), CSM14.1 (Haas et al., J Anat. 2002 July; 201(1):61-9), MN9D (Chen et al., Neurobiol Dis. 2005 August; 19(3):419-26), N27 cells (Kaul et al., J Biol Chem. 2005 Aug. 5; 280(31):28721-30), PC12 (Gorman et al., Biochem Biophys Res Commun. 2005 Feb. 18; 327(3):801-10), SN4741 (Nair et al., Biochem J. 2003 Jul. 1; 373(Pt 1):25-32), CHP-212, SH-SY5Y, and SK-N-BE.

### Test Agents and Extracts

In general, agents capable of modulating the selective elimination of defective mitochondria are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or agent is not critical to the screening procedure(s) of the invention. Agents used in screens may include known agents (for example, known therapeutics used for other diseases or disorders). Alternatively, virtually any number of unknown chemical extracts or agent can be screened using the methods described herein. Examples of such extracts or agents include, but are not limited to, plant-, fungal-, prokaryotic- or animal-based extracts, fermentation broths, and synthetic agents, as well as modification of existing agents.

Numerous methods are also available for generating random or directed synthesis (e.g., semi-synthesis or total synthesis) of any number of chemical agents, including, but not limited to, saccharide-, lipid-, peptide-, and nucleic acid-based agent. Synthetic compound libraries are commercially available from Brandon Associates (Merrimack, N.H.) and Aldrich Chemical (Milwaukee, Wis.). Alternatively, chemical agent to be used as candidate agent can be synthesized from readily available starting materials using standard synthetic techniques and methodologies known to those of ordinary skill in the art. Synthetic chemistry transformations and protecting group methodologies (protection and deprotection) useful in synthesizing the agent identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural agents in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of agents may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract of interest is identified, further fractionation of the positive lead extract is necessary to isolate chemical constituents responsible for the observed effect. Thus, the goal of the extraction, fractionation, and purification process is the careful characterization and identification of a chemical entity within the crude extract that alters the transcriptional activity of a gene associated with a mitochondrial disease. Methods of fractionation and purification of such heterogenous extracts are known in the art. If desired, agents shown to be useful as therapeutics for the treatment of a mitochondrial disease are chemically modified according to methods known in the art.

### Pharmaceutical Therapeutics

The invention provides agents that increase the expression or activity of Parkin or Pink1, including agents identified in the above-identified screens, for the treatment of a mitochondrial disease. In one embodiment, the invention provides pharmaceutical compositions comprising an expression vector encoding a Parkin or Pink1 polypeptide. In another embodiment, a chemical entity discovered to have medicinal value using the methods described herein is useful as a drug or as information for structural modification of existing agent, e.g., by rational drug design. For therapeutic uses, the compositions or agents identified using the methods disclosed herein may be administered systemically, for example, formulated in a pharmaceutically-acceptable carrier. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a mitochondrial disease therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and the clinical symptoms of the mitochondrial disease. Generally, amounts will be in the range of those used for other agents used in the treatment of a mitochondrial disease, although in certain instances lower amounts will be needed because of the increased specificity of the compound. A compound is administered at a dosage that controls the clinical or physiological symptoms of a mitochondrial disease as determined by a diagnostic method known to one skilled in the art, or using any that assay that measures the transcriptional activation of a gene associated with a mitochondrial disease.

### Formulation of Pharmaceutical Compositions

The administration of an agent of the invention or analog thereof for the treatment of a mitochondrial disease may be by any suitable means that results in a concentration of the therapeutic that, combined with other components, is effective in ameliorating, reducing, or stabilizing the mitochondrial disease or a symptom thereof. In one embodiment, administration of the agent reduces the percentage of defective mitochondria in a cell and/or increases the percentage of wild-type mitochondria. In one embodiment, the agent is administered to a subject for the prevention or treatment of a disease associated with mitochondrial dysfunction.

Methods of administering such agents are known in the art. The invention provides for the therapeutic administration of an agent by any means known in the art. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Suitable formulations include forms for oral administration, depot formulations, formulations for delivery by a patch, semisolid dosage forms to be topically or transdermally delivered.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a mitochondrial disease by using carriers or chemical derivatives to deliver the therapeutic agent to a particular cell type (e.g., cell having a mutation in mtDNA or a neuronal cell at risk of cell death) whose function is perturbed in the mitochondrial disease. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active therapeutic (s), the composition may include suitable parenterally acceptable carriers and/or excipients. The active therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle.

### Methods of Ocular Delivery

The compositions of the invention are also suitable for treating mitochondrial disease effecting the eye, such as LHON.

In one approach ocular delivery is achieved by injecting an agent of the invention directly into the eye. In another embodiment, the method involves the use of liposomes to target a compound of the present invention to the eye. For example, the compound may be complexed with liposomes, and this compound/liposome complex injected into patients with an ocular mitochondrial disease using intravenous injection to direct the compound to the desired ocular tissue or cell. In a specific embodiment, the compound is administered via intra-ocular sustained delivery (such as VITRASERT or ENVISION). In a specific embodiment, the compound is delivered by posterior subtenons injection. In another specific embodiment, microemulsion particles containing the compositions of the invention are delivered to ocular tissue.

In one approach, the compositions of the invention are administered through an ocular device suitable for direct implantation into the vitreous of the eye. The compositions of the invention may be provided in sustained release compositions, such as those described in, for example, U.S. Pat. Nos. 5,672,659 and 5,595,760. Such devices are found to provide sustained controlled release of various compositions to treat the eye without risk of detrimental local and systemic side effects. An object of the present ocular method of delivery is to maximize the amount of drug contained in an intraocular device or implant while minimizing its size in order to prolong the duration of the implant. See, e.g., U.S. Pat. Nos. 5,378,475; 6,375,972, and 6,756,058 and U.S. Publications 20050096290 and 200501269448. Such implants may be biodegradable and/or biocompatible implants, or may be non-biodegradable implants. Biodegradable ocular implants are described, for example, in U.S. Patent Publication No. 20050048099. The implants may be permeable or impermeable to the active agent, and may be inserted into a chamber of the eye, such as the anterior or posterior chambers or may be implanted in the schlera, transchoroidal space, or an avascularized region exterior to the vitreous. Alternatively, a contact lens that acts as a depot for compositions of the invention may also be used for drug delivery.

In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula. Examples of implants for delivery of an a composition include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

Examples include, but are not limited to the following: a sustained release drug delivery system comprising an inner reservoir comprising an effective amount of an agent effective in obtaining a desired local or systemic physiological or pharmacological effect, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end; a method for administering a compound of the invention to a segment of an eye, the method comprising the step of implanting a sustained release device to deliver the compound of the invention to the vitreous of the eye or an implantable, sustained release device for administering a compound of the invention to a segment of an eye; a sustained release drug delivery device comprising: a) a drug core comprising a therapeutically effective amount of at least one first agent effective in obtaining a diagnostic effect or effective in obtaining a desired local or systemic physiological or pharmacological effect; b) at least one unitary cup essentially impermeable to the passage of the agent that surrounds and defines an internal compartment to accept the drug core, the unitary cup comprising an open top end with at least one recessed groove around at least some portion of the open top end of the unitary cup; c) a permeable plug which is permeable to the passage of the agent, the permeable plug is positioned at the open top end of the unitary cup where the groove interacts with the permeable plug holding it in position and closing the open top end, the permeable plug allowing passage of the agent out of the drug core, through the permeable plug, and out the open top end of the unitary cup; and d) at least one second agent effective in obtaining a diagnostic effect or effective in obtaining a desired local or systemic physiological or pharmacological effect; or a sustained release drug delivery device comprising: an inner core comprising an effective amount of an agent having a desired solubility and a polymer coating layer, the polymer layer being permeable to the agent, where the polymer coating layer completely covers the inner core.

### Controlled Release Parenteral Compositions

Controlled release parenteral compositions may be in the form of suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing an active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active mitochondrial disease therapeutic substance). The coating may be applied on the solid dosage form in a similar manner as that described in Encyclopedia of Pharmaceutical Technology, supra.

At least two active mitochondrial disease therapeutics may be mixed together in the tablet, or may be partitioned. In one example, the first active therapeutic is contained on the inside of the tablet, and the second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may be constructed to release the active mitochondrial disease therapeutic by controlling the dissolution and/or the diffusion of the active substance. Dissolution or diffusion controlled release can be achieved by appropriate coating of a tablet, capsule, pellet, or granulate formulation of agent, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic agent may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Topical Administration Forms

Dosage forms for the semisolid topical administration of an agent of this invention include ointments, pastes, creams, lotions, and gels. The dosage forms may be formulated with mucoadhesive polymers for sustained release of active ingredients at the area of application to the skin. The active compound may be mixed under sterile conditions with a pharmaceutically acceptable carrier, and with any preservatives, buffers, or propellants, which may be required. Such topical preparations can be prepared by combining the compound of interest with conventional pharmaceutical diluents and carriers commonly used in topical liquid, cream, and gel formulations.

Ointments and creams may, for example, be formulated with an aqueous or oily base with the addition of suitable thickening and/or gelling agents. Such bases may include water and/or an oil (e.g., liquid paraffin, vegetable oil, such as peanut oil or castor oil). Thickening agents that may be used according to the nature of the base include soft paraffin, aluminum stearate, cetostearyl alcohol, propylene glycol, polyethylene glycols, woolfat, hydrogenated lanolin, beeswax, and the like.

Lotions may be formulated with an aqueous or oily base and, in general, also include one or more of the following: stabilizing agents, emulsifying agents, dispersing agents, suspending agents, thickening agents, coloring agents, perfumes, and the like. The ointments, pastes, creams and gels also may contain excipients, including, but not limited to, animal and vegetable fats, oils, waxes, paraffins, starch, tragacanth, cellulose derivatives, polyethylene glycols, silicones, bentonites, silicic acid, talc and zinc oxide, or mixtures thereof.

Suitable excipients, depending on the hormone, include petrolatum, lanolin, methylcellulose, sodium carboxymethylcellulose, hydroxpropylcellulose, sodium alginate, carbomers, glycerin, glycols, oils, glycerol, benzoates, parabens and surfactants. It will be apparent to those of skill in the art that the solubility of a particular compound will, in part, determine how the compound is formulated. An aqueous gel formulation is suitable for water soluble agent. Where a compound is insoluble in water at the concentrations required for activity, a cream or ointment preparation will typically be preferable. In this case, oil phase, aqueous/organic phase and surfactant may be required to prepare the formulations. Thus, based on the solubility and excipient-active interaction information, the dosage forms can be designed and excipients can be chosen to formulate the prototype preparations.

The topical pharmaceutical compositions can also include one or more preservatives or bacteriostatic agents, e.g., methyl hydroxybenzoate, propyl hydroxybenzoate, chlorocresol, benzalkonium chlorides, and the like. The topical pharmaceutical compositions also can contain other active ingredients including, but not limited to, antimicrobial agents, particularly antibiotics, anesthetics, analgesics, and antipruritic agents.

### Dosage

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Therapeutic Methods

The present invention provides methods of treating a mitochondrial disease or symptoms thereof (e.g., cytotoxicity) by modulating the selective elimination of defective mitochondria. The methods comprise administering a therapeutically effective amount of a pharmaceutical composition comprising a compound that modulates the selective elimination of defective mitochondria using the methods described herein to a subject (e.g., a mammal such as a human). Thus, one embodiment is a method of treating a subject suffering from or susceptible to a mitochondrial disease or symptom thereof. The method includes the step of administering to the subject a therapeutic amount of an amount of a compound herein sufficient to treat the disease or symptom thereof, under conditions such that the disease is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention, which include prophylactic treatment, in general comprise administration of a therapeutically effective amount of the agent herein, such as a compound of the formulae herein to a subject (e.g., animal, human) in need thereof, including a mammal, particularly a human. Such treatment will be suitably administered to subjects, particularly humans, suffering from, having, susceptible to, or at risk for a mitochondrial disease or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agent herein may be also used in the treatment of any other disorders in which transcriptional activity may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a mitochondrial disease, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The following examples are provided to illustrate the invention, not to limit it. Those skilled in the art will understand that the specific constructions provided below may be changed in numerous ways, consistent with the above described invention while retaining the critical properties of the agent or combinations thereof.

### Kits

The invention provides kits for the treatment or prevention of a disease associated with mitochondrial dysfunction. In one embodiment, the kit includes a therapeutic or prophylactic composition containing an effective amount of an agent of the invention (e.g., a vector encoding Pink1, Parkin) in unit dosage form. In some embodiments, the kit comprises a sterile container which contains a therapeutic or prophylactic compound; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering it to a subject having or at risk of developing a mitochondrial disorder. The instructions will generally include information about the use of the composition for the treatment or prevention of the mitochondrial disorder. In other embodiments, the instructions include at least one of the following: description of the compound; dosage schedule and administration for treatment or prevention of a mitochondrial disorder or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Combination Therapies

Optionally, an agent having therapeutic or prophylactic efficacy may be administered in combination with any other standard therapy for the treatment of a mitochondrial disease; such methods are known to the skilled artisan and described in Remington's Pharmaceutical Sciences by E. W. Martin. If desired, agents of the invention may be administered alone or in combination with a conventional therapeutic useful for the treatment of a mitochondrial disease. Therapeutics useful for the treatment of Parkinson's disease include, but are not limited to, deprenyl, amantadine or anticholinergic medications, levodopa, carbidopa, entacapone, pramipexole, rasagiline, antihistamines, antidepressants, dopamine agonists, monoamine oxidase inhibitors (MAOIs), and others.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Defective Mitochondria are Selectively Targeted by Parkin

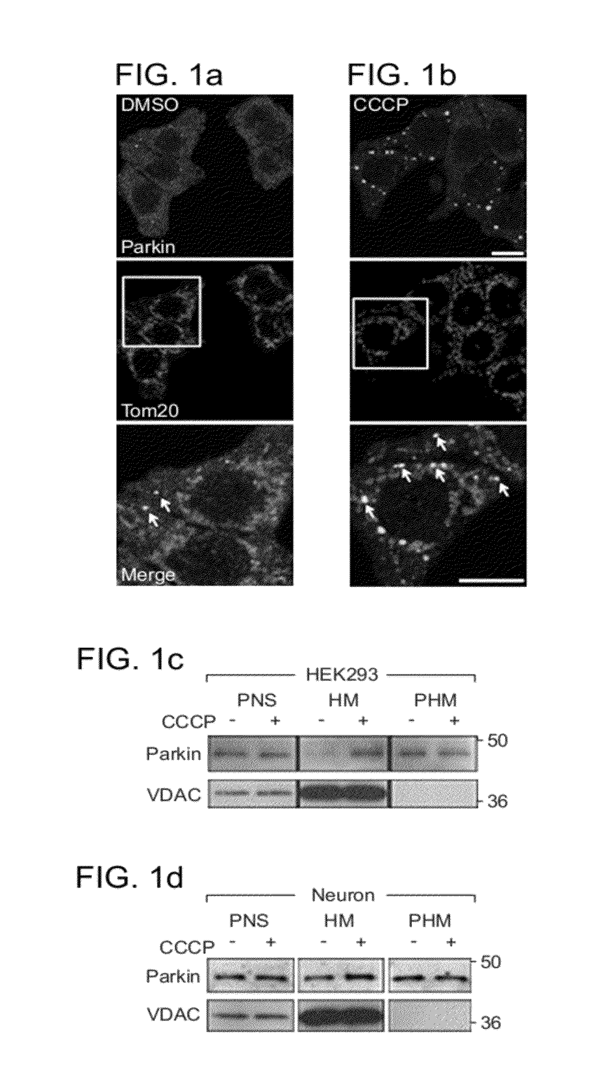

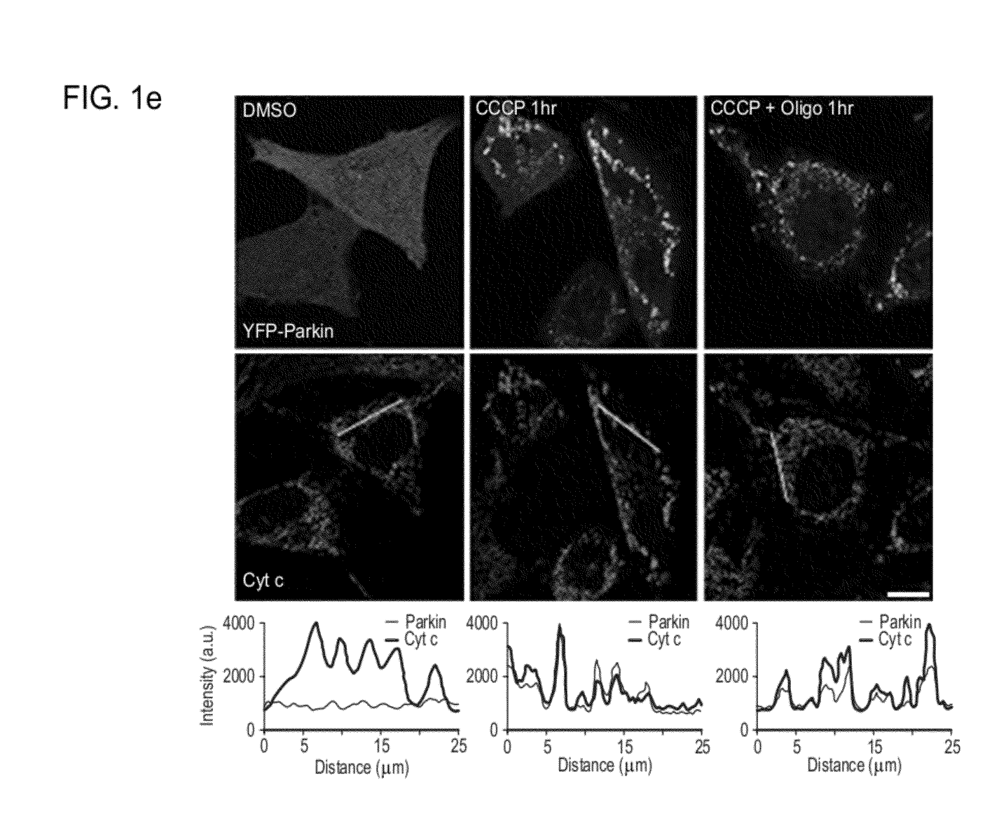

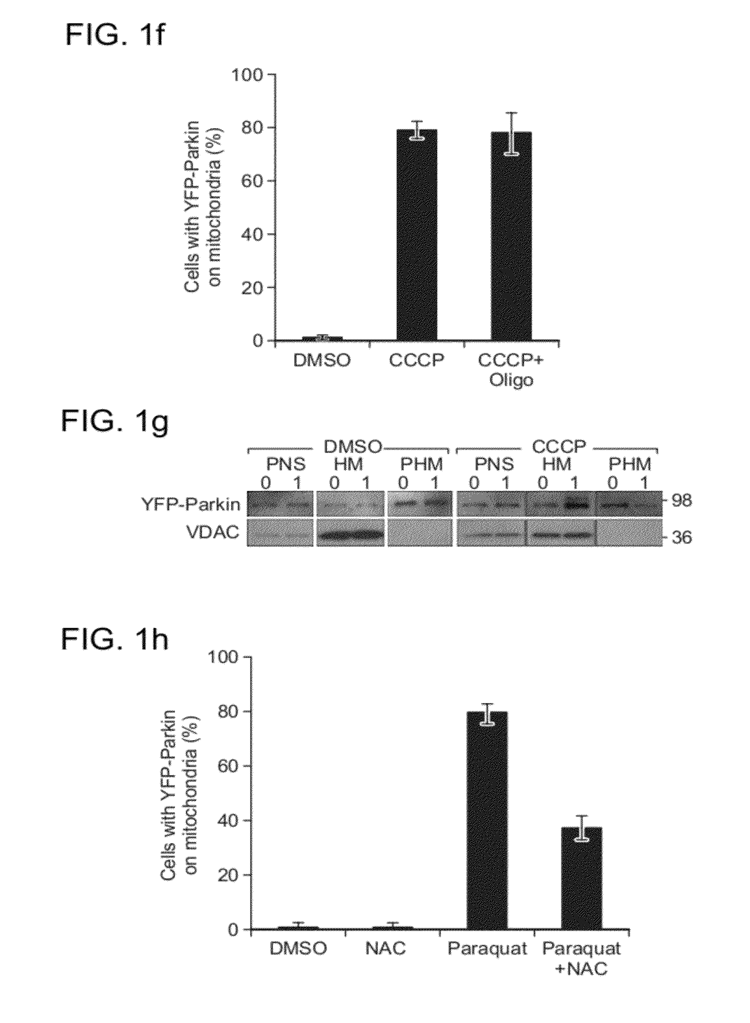

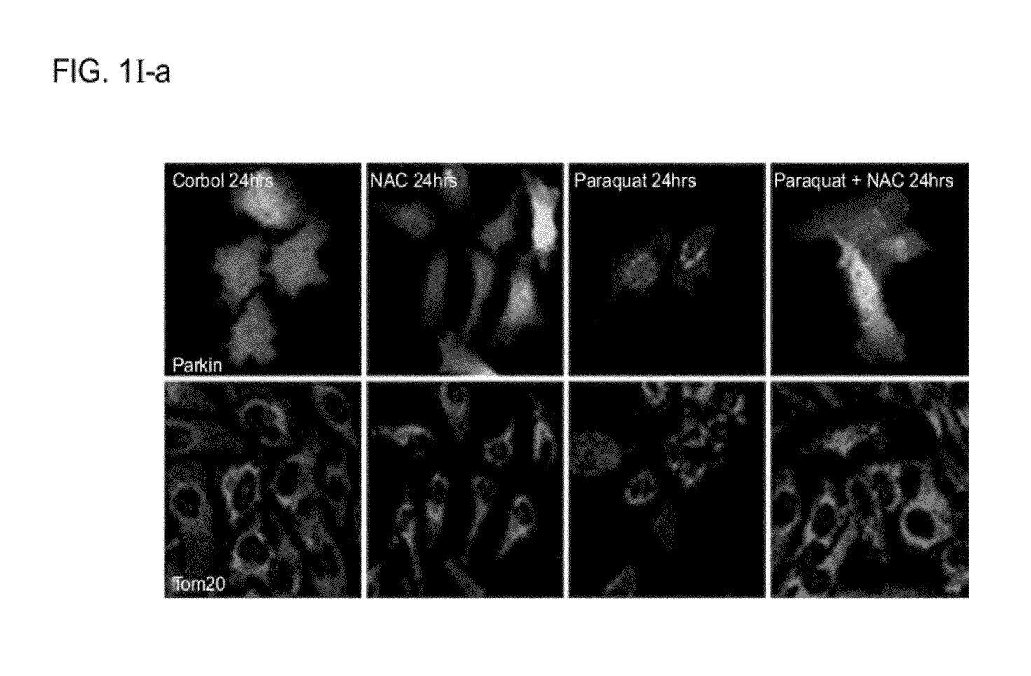

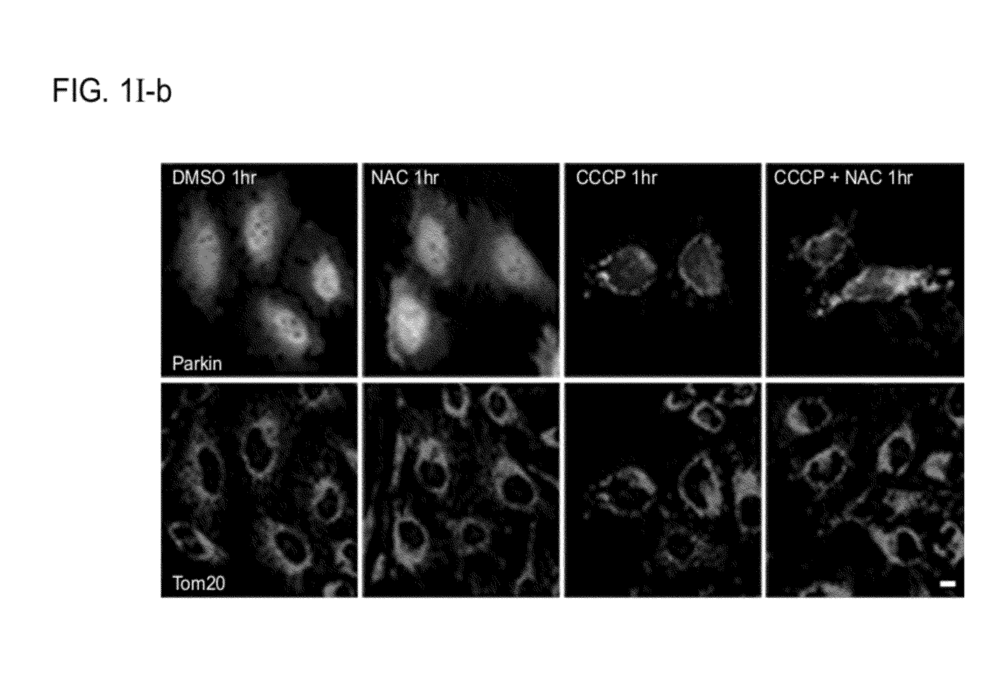

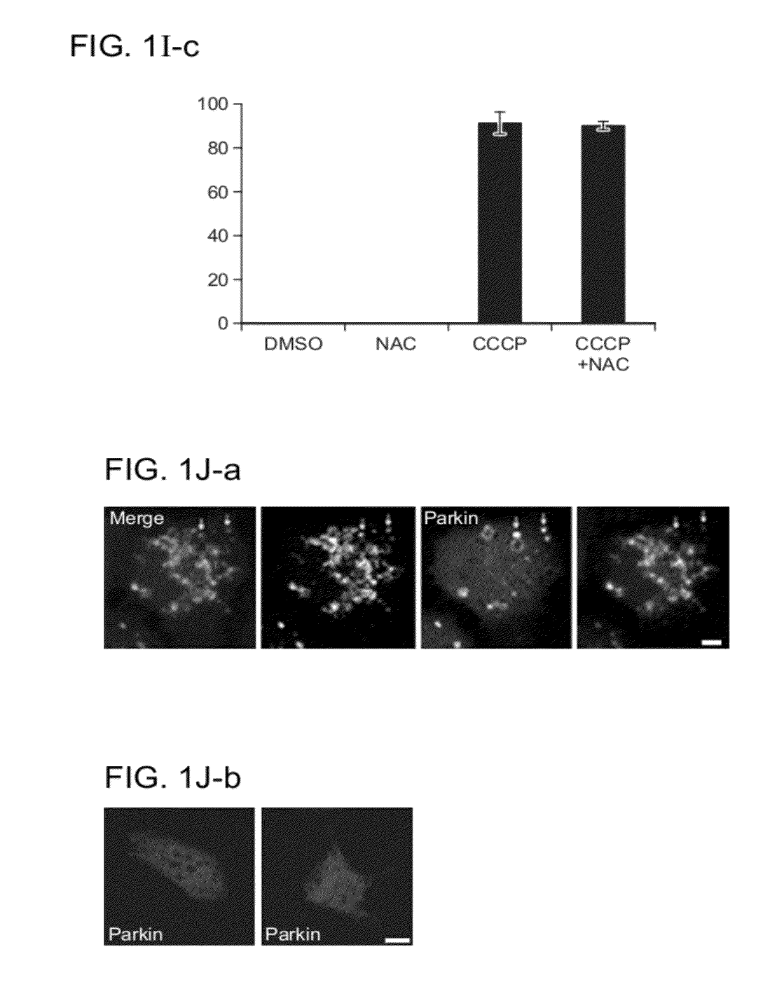

Parkin subcellular localization findings by others show conflicting results indicating the protein in the cytosol or associated with ER or mitochondria. The subcellular localization of endogenous Parkin was examined in HEK293 cells, a cell line that expresses relatively high levels of Parkin, using the PRK8 monoclonal antibody (Pawlyk et al., J. Biol. Chem. 278: 48120-48128, 2003). Consistent with most studies, endogenous Parkin was predominately located in the cytosol (FIGS. 1A and 1c). However, in some of the cells, colocalization was observed between Parkin and a subset of the mitochondria, which were small and fragmented (FIG. 1A).

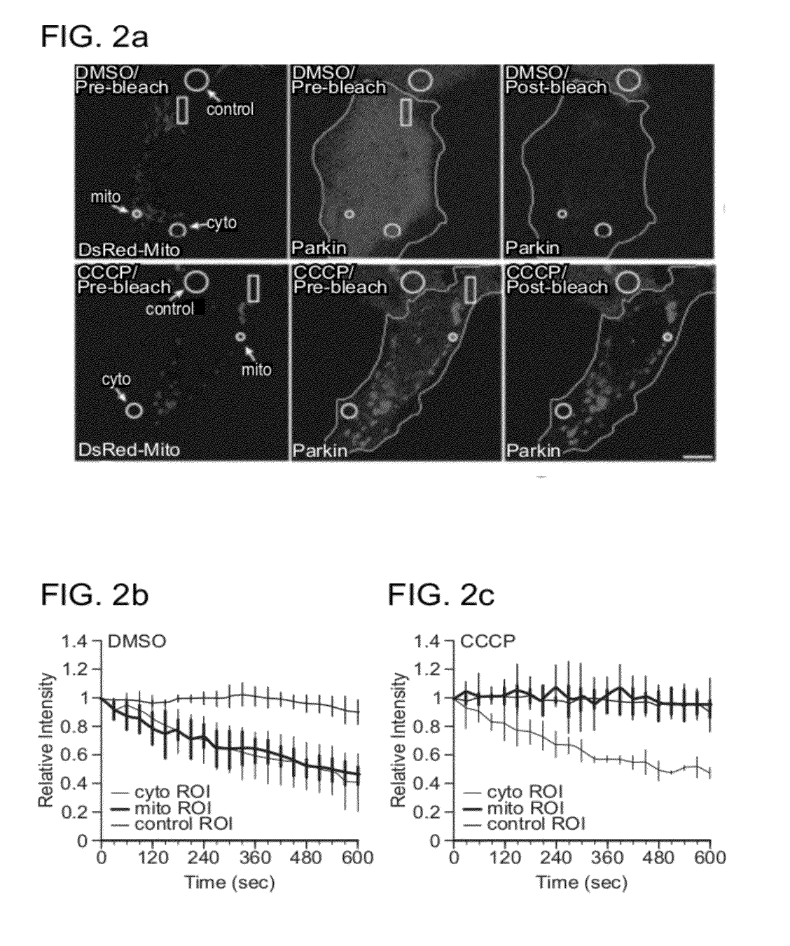

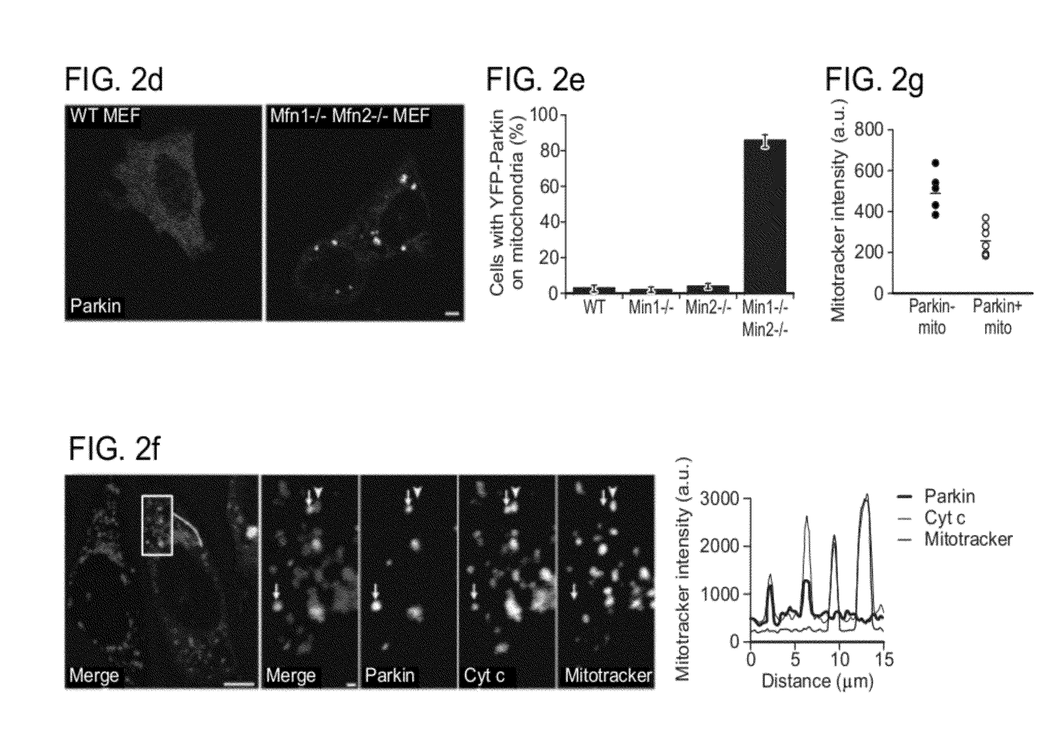

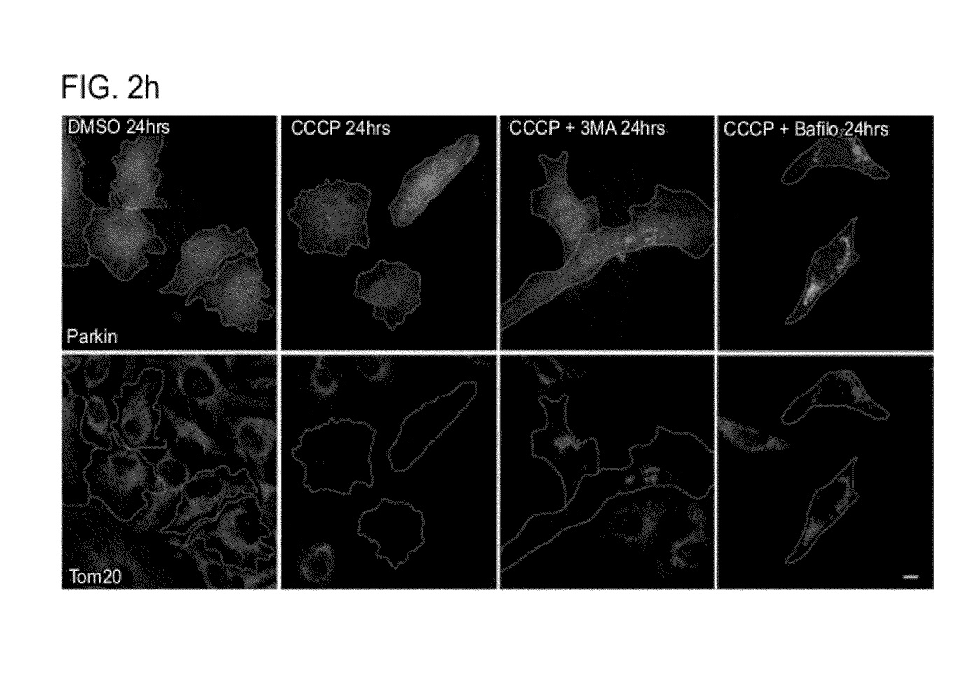

Mitochondrial fission has been linked to the function of Parkin and to the autophagy of small defective mitochondria that lack membrane potential. To test whether mitochondrial depolarization causes Parkin accumulation on mitochondria, HEK293 cells were treated with the mitochondrial uncoupler carbonyl cyanide m-chlorophenylhydrazone (CCCP). Within 1 hour of adding CCCP, endogenous Parkin was recruited to mitochondria in the majority of cells (FIG. 1B) and increased appearance in the heavy membrane pellet on Western blots (FIG. 1C). Although rat cortical neuron cultures displayed more Parkin in the membrane pellet. than did HEK293 cells, uncoupling of mitochondria with CCCP increased levels in the membrane pellet (FIG. 1D). YFP-Parkin expressed in HeLa cells, which have little or no endogenous Parkin expression, displayed a cytosolic distribution in >99% of cells. As with endogenous Parkin in HEK293 cells, CCCP exposure induced the redistribution of YFP-Parkin from the cytosol to the mitochondria (FIGS. 1E and F). This CCCP-induced accumulation of Parkin on mitochondria was not inhibited by the addition of the ATP synthase inhibitor oligomycin (which decreases ATP consumption by mitochondrial uncouplers; 78.49±2.61% [mean±SD] with CCCP alone vs. 77.35±7.64% with CCCP+ oligomycin; FIGS. 1E and 1F). Western blots also show that YFP-Parkin redistributes from the cytosol to the heavy membrane pellet upon CCCP treatment (FIG. 1G). Additionally, YFP-Parkin was recruited to depolarized mitochondria damaged by the pesticide paraquat, which is thought to increase complex I-dependent reactive oxygen species and has been linked to Parkinsonism (FIGS. 1H, 1I, and 1J). CCCP-induced recruitment was not blocked by the antioxidant N-acetyl-cysteine, which suggests that reactive oxygen species production is not necessary for Parkin translocation (FIG. 1I-a, 1I-b, 1H-c, FIG. 1J-a, 1J-b). The mitochondrial translocation of Parkin caused by mitochondrial depolarization was also assayed by fluorescence loss in photobleaching (FLIP). Mitochondrial-localized YFP-Parkin in CCCP-treated cells was depleted more slowly by photobleaching than the entire pool of YFP-Parkin in HeLa cells not exposed to CCCP, which suggests that YFP-Parkin's affinity for mitochondria is increased upon depolarization (FIGS. 2A-2C).

Chronic inhibition of mitochondrial fusion caused by double knockout of the genes expressing the partially redundant mitofusin (Mfn) proteins, Mfn1 and Mfn2, generates a heterogenous population of fragmented mitochondria, some of which are relatively respiratory deficient and display a lower membrane potential. If Parkin recruitment occurs as a consequence of membrane depolarization, exogenous Parkin in Mfn1−/−, Mfn2−/− double knockout mouse embryonic fibroblasts (MEFs) would be predicted to accumulate selectively on mitochondria with lower membrane potentials. YFP-Parkin colocalized with mitochondria in 1.33±1.15% of Mfn1−/− cells and 3.33±1.15% of Mfn2−/− cells, in the range of the 1.99±2% of cells displaying Parkin-positive mitochondria seen with wild-type (WT) MEFs. However, in Mfn1−/−, Mfn2−/− double knockout MEFs, YFP-Parkin colocalized with mitochondria in 86.20±3.95% of cells (FIGS. 2D and 2E; and FIG. 2H; P<0.001 for Mfn1−/−, Mfn2−/− vs. WT [two-tailed t test]). Interestingly, in Mfn1−/−, Mfn2−/− cells, Parkin was recruited to a discreet subset of mitochondria within individual cells (FIGS. 2D and 2F). To test whether mitochondria labeled by Parkin display decreased membrane potential, the cells were pulsed with MitoTracker red, a potentiometric mitochondrial dye, before fixation. YFP-Parkin selectively accumulated on those mitochondria with lower MitoTracker staining (FIG. 2F). To quantify this relationship, the mitochondrial volume of these cells was digitally segregated into Parkin-positive and Parkin-negative sets, and measured the mean MitoTracker intensity of these volumes for each cell. The mitochondrial volume labeled with YFPParkin displayed a 47% lower mean MitoTracker intensity relative to the mitochondrial volume with undetectable YFP-Parkin accumulation (FIG. 2G; 487.00±81.5 arbitrary units [au] vs. 258.3±61.7 au; P<0.001 [two-tailed, paired t test], n=9 cells). These results show that Parkin can be recruited to individual mitochondria within cells and that compromised mitochondria display greater Parkin accumulation than electrochemically active mitochondria, which is consistent with the hypothesis that impaired mitochondria are selectively targeted by Parkin.

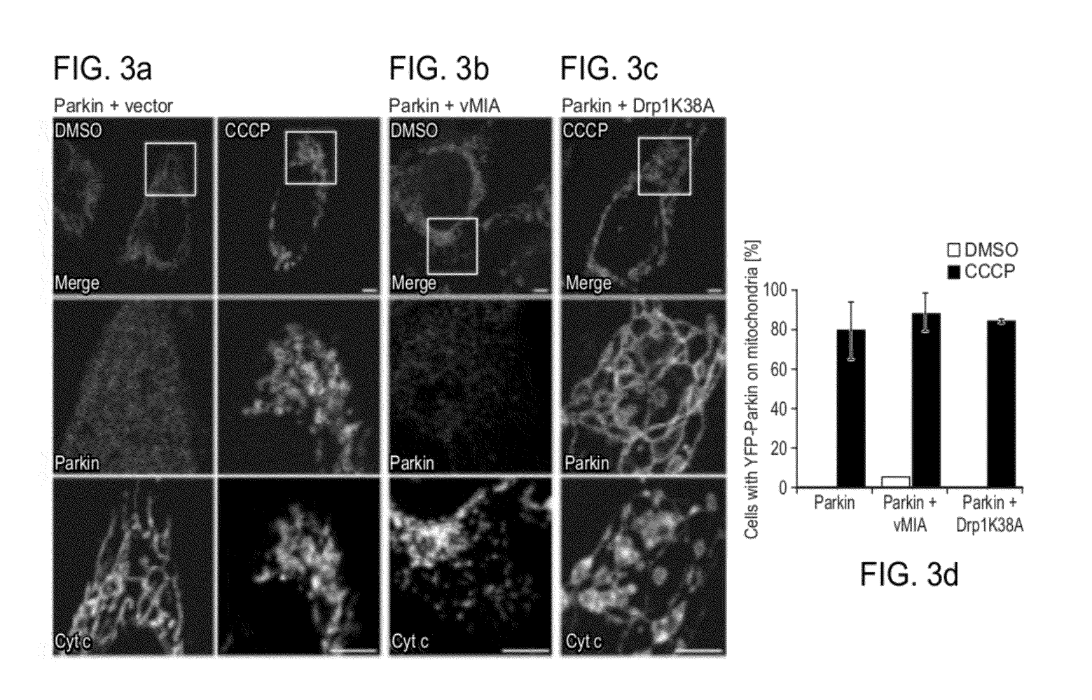

Depolarization of mitochondria is known to induce their fragmentation into multiple smaller organelles by inhibiting organelle fusion. Recent genetic studies have linked Parkin activity to gene products controlling mitochondrial fission and fusion. To determine whether Parkin recruitment to mitochondria may be a consequence of depolarization induced fragmentation, fragmentation of mitochondria induced by CCCP (FIG. 3A) was inhibited by overexpressing Drp1K38A, a dominant-negative mutant of the mitochondrial fission protein dynamin-related protein 1 (Drp1; FIG. 3C; Smirnova et al., Mol. Biol. Cell. 12: 2245-2256, 2001). Although mitochondria in CCCP-treated cells expressing Drp1K38A fail to fragment, they still display Parkin accumulation along the elongated mitochondria (FIGS. 3C and 3D), which indicates that mitochondrial fragmentation is not necessary for Parkin translocation. Expression of viral mitochondrial associated inhibitor of apoptosis (vMIA) in HeLa cells, which causes fragmentation of mitochondria with minimal perturbation of membrane potential (McCormick et al., 2003), did not cause accumulation of Parkin on mitochondria (FIGS. 3B and 3D), also indicating that excessive fragmentation of mitochondria by itself is insufficient to cause Parkin recruitment.

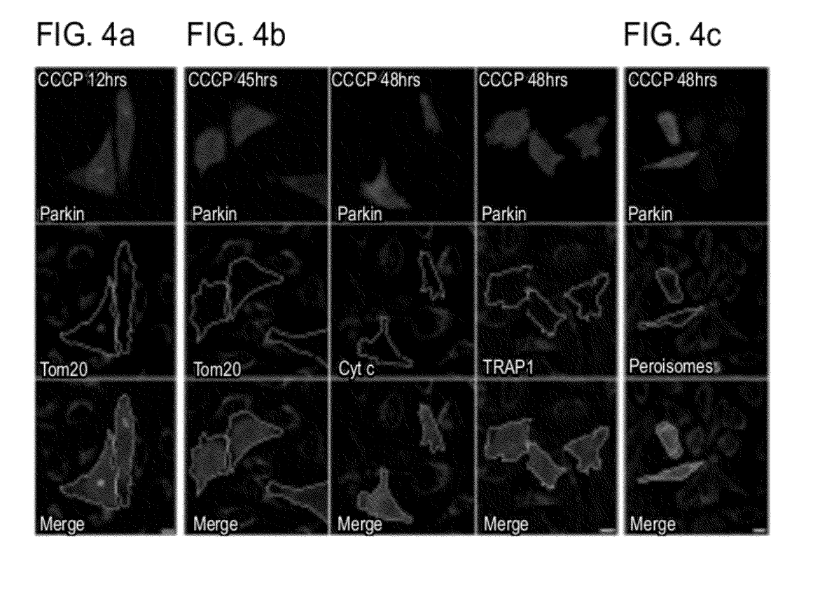

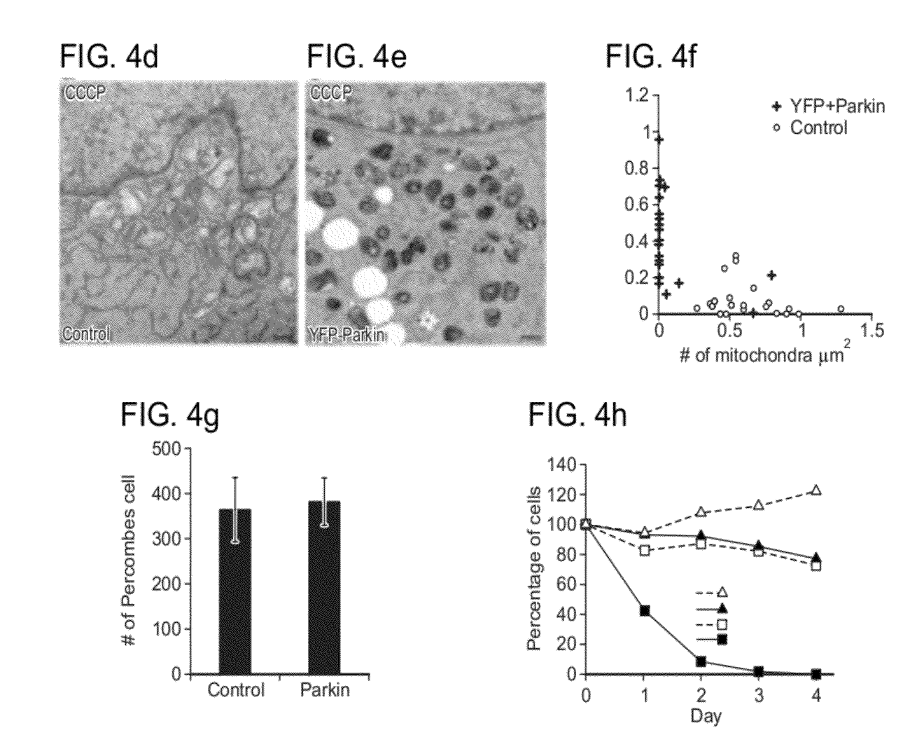

To further assess the effect of Parkin on depolarized mitochondria, changes in mitochondrial morphology and mass over time were followed. In HeLa cells lacking Parkin, the mitochondria appeared fragmented within 60 minutes after adding CCCP but underwent little other morphological change over the following 48 h (FIGS. 4A and 4B). In Parkin-expressing cells, in contrast, the mitochondrial mass appeared to be grossly reduced by 12 hours (FIG. 4 a). Interestingly, by 48 hours, no mitochondria remained detectable in Parkin-expressing cells assessed by immunocytochemistry using three independent mitochondria markers: Tom20, cytochrome c, and TRAP1 (FIG. 4B). In contrast to the mitochondrial elimination, no significant decrease in the number of peroxisomes was observed (FIGS. 4C and 4G), which suggests that Parkin selectively induces mitophagy that is consistent with the mitochondria-specific localization upon CCCP treatment.

HeLa cell mitochondria were also examined by transmission electron microscopy after 48 hours of CCCP treatment in the presence and absence of Parkin expression. Mitochondria were abundant in the control HeLa cells after CCCP treatment, although they appeared fragmented and had sparse cristae (FIG. 4D). However, 90% of HeLa cells expressing YFP-Parkin had either few or no detectable mitochondrial structures (FIGS. 4E and F; 0.074±0.21 mitochondria/μm 2 of cytoplasm with Parkin vs. 0.62±0.06 mitochondria/μm 2 of cytoplasm without Parkin; P<0.001, n=22 cells per condition). Furthermore, Parkin-expressing HeLa cells lacking mitochondria displayed a large increase in electron dense lysosomal structures (FIGS. 4E and F; 0.38±0.23 lysosomes/μm 2 cell area with Parkin vs. 0.06±0.08 lysosomes/μm 2 of cytoplasm without Parkin; P<0.001, n=22 cells per condition).

To further confirm that cells had lost their mitochondria, their growth in glucose media or galactose media, which lacked glucose, was examined. 72.8% of cells without detectable mitochondria were able to survive for 4 days in glucose media, whereas 0% were able to survive 4 days when cultured in galactose media (FIG. 4H). In contrast, the majority of control cells, which had been treated with CCCP but lacked Parkin, retained their mitochondria and could survive in both glucose and galactose media for at least 4 days (FIG. 4H). These results provide biochemical evidence that cells expressing Parkin lack mitochondrial function after depolarization, which is consistent with their having been eliminated.

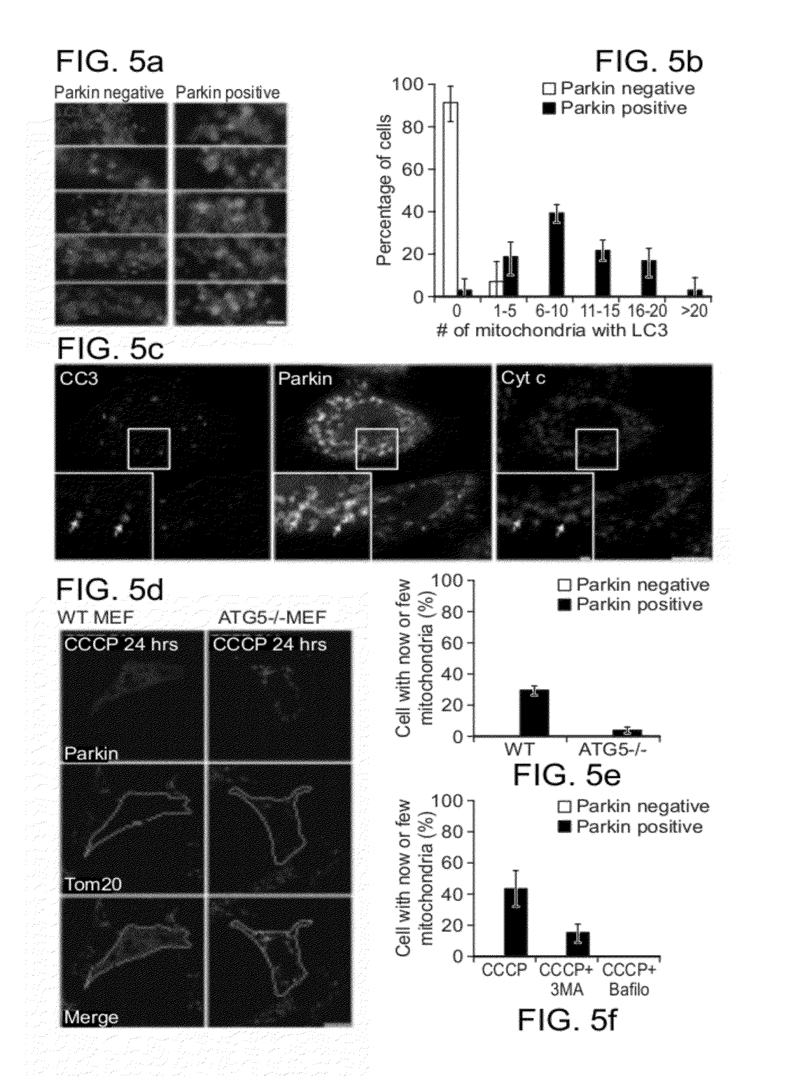

Previous studies in mammalian cells have concluded that depolarized mitochondria are degraded by autophagy. To test whether Parkin may be regulating this process, colocalization was assessed between a marker of autophagosomes, LC3, and mitochondria after mitochondrial depolarization using HeLa cells stably transfected with GFP-LC3 (Bampton et al., 2005). Little colocalization between mitochondria and autophagosomes was seen after 1 hours of CCCP exposure in untransfected HeLa cells (FIG. 5A, left). However, LC3-labeled structures surrounded fragmented mitochondria in cells transfected with mCherry-Parkin specifically after CCCP treatment (FIG. 5A, right) to a significantly greater extent than in the Parkin-deficient HeLa cells (10.55±6.06 vs. 0.09±0.36 LC3 encompassed mitochondria per cell; two-sided t test, P<0.001; FIG. 5b). Consistent with the conclusion that Parkin accumulates on mitochondria destined for autophagy, Parkin colocalized with LC3 after CCCP treatment (FIG. 5C) but not before.

To experimentally test if Parkin mediated mitochondrial elimination by autophagy, Parkin activity in ATG5−/− MEFs that lack a key component of the autophagy pathway was examined (Hara et al., 2006). Supporting the hypothesis that Parkin promotes autophagic degradation of impaired mitochondria, cells lacking ATG5 retain Parkin-targeted mitochondria after CCCP treatment (FIGS. 5D and 5e). Likewise, bafilomycin, a lysosomal inhibitor, and 3-methyl adenine, an inhibitor of autophagy, blocked Parkin induced mitophagy in HeLa cells (FIGS. 5F and 2H).

As reported above, Parkin is recruited to depolarized mitochondria and Parkin promotes their autophagic degradation. Spontaneous mitochondrial depolarization and depolarization after phototoxicity have been associated with mitophagy in mammalian cells. Although little is known about the proteins regulating this process in mammalian cells, recently, BNIP3L/NIX was found to promote degradation of mitochondria in reticulocytes by triggering the loss of mitochondrial membrane potential. These findings provide a new molecular link between mitochondrial membrane depolarization and autophagy by identifying Parkin as a mediator of mitophagy downstream of mitochondrial depolarization.

Long-lived cells may require greater mitochondrial quality control than dividing cell populations that can discard damaged mitochondria wholesale by eliminating defective cells. Thus, certain cell types, such as neurons and myocytes, may require more robust intracellular mitochondrial surveillance than proliferating cell populations.

In D. melanogaster, knockout of mitochondrial fusion genes can partially compensate for loss of Parkin phenotypes. The results provided herein support the view that Parkin has a less direct mode of compensating for defects in mitochondrial fusion and fission. Mitochondrial fragmentation does not itself signal Parkin recruitment, but a severe defect in mitochondrial fusion does trigger recruitment of Parkin to mitochondria if they lose membrane potential. Additionally, mitochondrial fission appears to be a prerequisite for mitophagy. Thus, excess fission may compensate for Parkin loss in the fly by promoting mitophagy.

Parkin overexpression also has been shown to compensate for loss of Pink1 in D. melanogaster. The results reported herein suggest that Parkin may compensate by targeting impaired Pink1-deficient mitochondria for degradation. Knockdown of Pink1 leads to reduced HeLa cell mitochondrial membrane potential, which suggests that Parkin could maintain fidelity of mitochondria by activating the autophagy of dysfunctional mitochondria resulting from Pink1 loss.

Most importantly, these results suggest that loss of Parkin activity may allow the accumulation of dysfunctional mitochondria, leading to neuron loss in Parkinson's disease, and that Parkin normally functions to survey mitochondrial activity and maintain mitochondrial fidelity by activating the autophagy of damaged organelles.

### Example 2

### PINK1 Accumulates Following Mitochondrial Depolarization

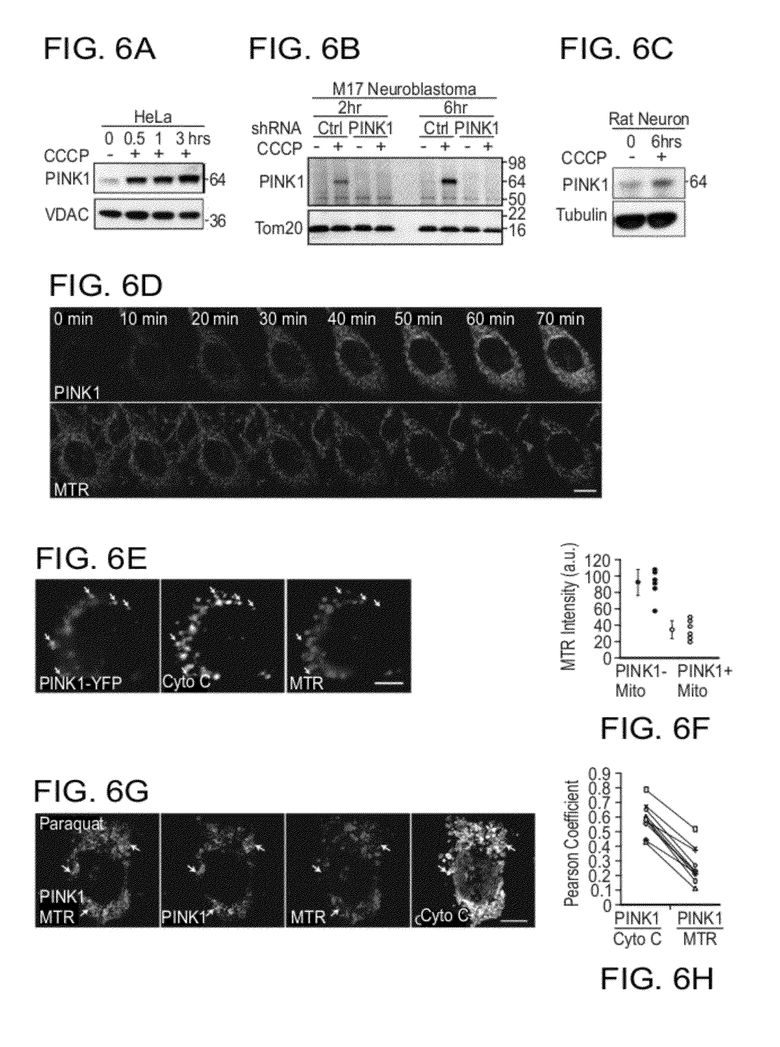

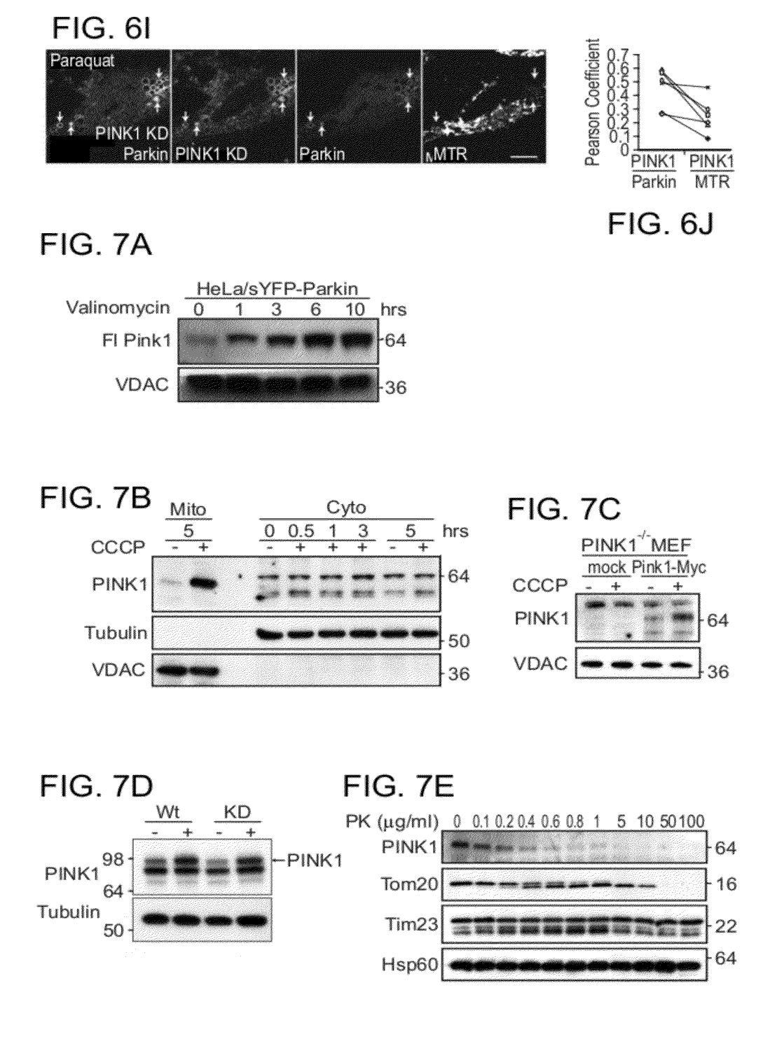

Parkin is selectively recruited to damaged mitochondria that have lost their membrane potential, but how Parkin distinguishes dysfunctional mitochondria with low membrane potential from healthy mitochondria is unknown. Since PINK1 is genetically upstream of Parkin, PINK1's activity might be activated by mitochondrial depolarization. This hypothesis was tested. Remarkably, levels of endogenous mitochondrial PINK1 respond robustly to changes in mitochondrial membrane potential. When HeLa cells are depolarized with CCCP, a large increase in endogenous full length PINK1 (˜63 kDa) is seen beginning by 30 minutes and continuing for at least three hours (FIG. 6A). This ˜63 kDa band also increased in the mitochondrial-rich membrane fraction following treatment with valinomycin, which depolarizes mitochondria by a different mechanism (FIG. 7A). By contrast, no band increases were observed in the cytosolic fraction following depolarization with CCCP ((FIG. 7B).

To verify that the ˜63 kDa band is in fact PINK1, M17 cells stably transduced with control shRNA or PINK shRNA were immunoblotted for endogenous PINK1 in. The ˜63 kDa band increased following CCCP treatment in control shRNA cells, but did not increase in the PINK1 shRNA cells, demonstrating that this ˜63 kDa band is endogenous PINK1 (FIG. 6B). Similar results were found in PINK1−/− cells transfected with PINK1-myc or left untransfected. The question of whether PINK1 similarly accumulates in primary rat cortical neurons following depolarization with CCCP was explored. These experiments did not detect endogenous rat or mouse PINK1 with the available commercial antibodies. PINK1-V5 increases in cortical neurons following treatment with 1 μM of CCCP for 6 hours. With CCCP treatment, PINK1 may accumulate more slowly in primary neurons than HeLa cells, because, unlike HeLa cells, neurons rely almost exclusively on respiration for ATP production.

To explore the kinetics of PINK1 accumulation at the single cell level, YFP was fused to PINK1 and cells were imaged live following depolarization with CCCP. Consistent with results obtained by Western blotting, PINK1-YFP expression steadily increased from 1-5 minutes, when an increase was first detectable, until at least 70 minutes (FIG. 6D).

### PINK1 Accumulates Preferentially on Depolarized Mitochondria in a Single Cell

To examine the selectivity of PINK1 accumulation on uncoupled mitochondria within single cells, its expression in MEFs null for mitochondrial fusion proteins mitofusin-1 and mitofusin-2 (Mfn1/2) was examined. The Mfn1/2 null MEFs have a heterogenous population of mitochondria, some of which are bioenergetically uncoupled and some of which are well-coupled (Chen et al., J Biol Chem 280: 26185-26192, 2005). Similar to YFPParkin, PINK1-YFP accumulated selectively on mitochondria with low membrane potential, demonstrating that PINK1 is selectively stabilized on the depolarized mitochondria within a bioenergetically diverse population of mitochondria (FIGS. 6E and 6F).

Treatment with paraquat, a pesticide that has been linked to Parkinsonism, also resulted in a heterogeneous population of mitochondria, likely due to stochastic damage of mitochondria by reactive oxygen species. HeLa cells were treated overnight with a high dose of paraquat (2 mM). Similar to results with Parkin reported previously (Chen et al., J Biol Chem 280: 26185-26192, 2005), PINK1-YFP accumulated preferentially on damaged mitochondria with low membrane potential (FIG. 6G). While PINK1-YFP co-localizes with cytochrome c, which is present in all mitochondria (average pearson coefficient=0.58±0.11), PINK1-YFP does not co-localize with MTR (average pearson coefficient 0.26±0.13), which accumulates only in bioenergetically active mitochondria (p-value<0.001 for PINK1/cytochrome c vs. PINK1/MTR, paired Student's t-test). These data suggest that PINK1-YFP accumulates selectively on depolarized mitochondria that have been damaged by oxidative stress (FIG. 6H).

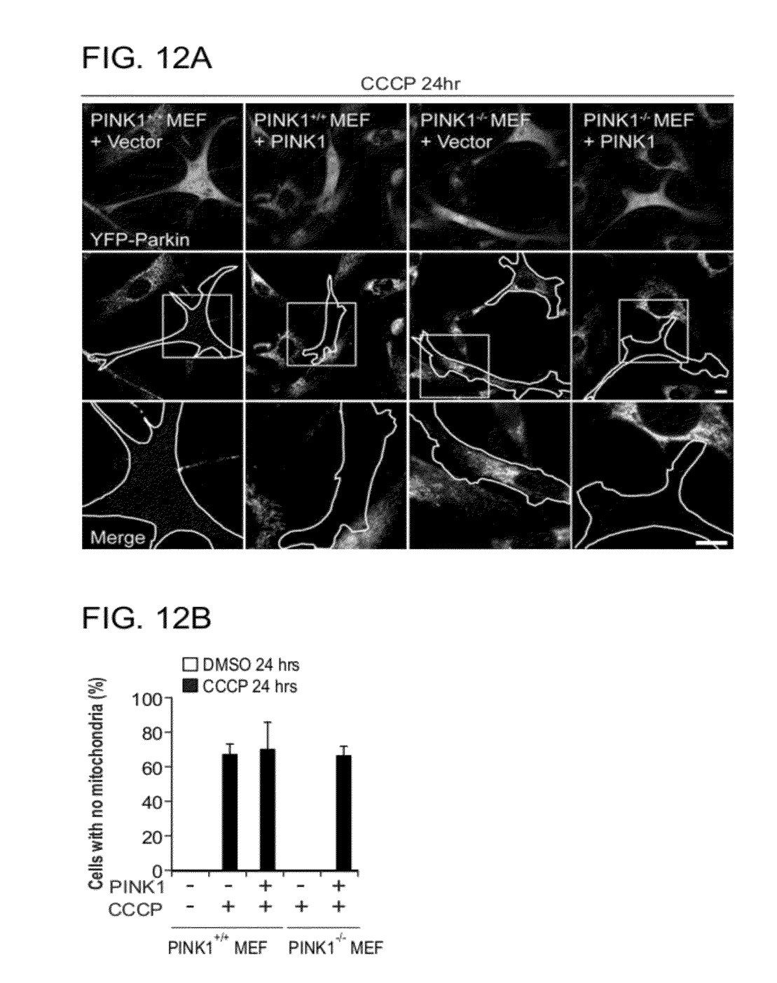

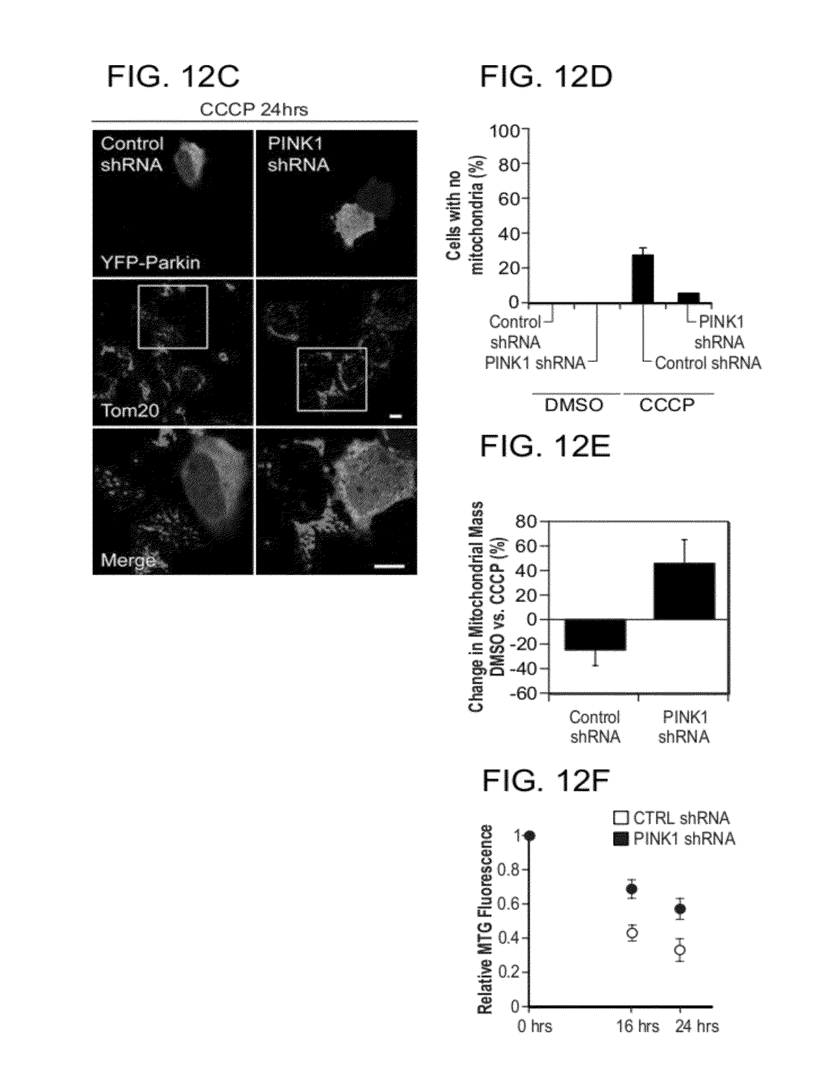

Next, the question of whether Parkin is recruited to the same depolarized mitochondria that accumulate PINK1 following treatment with paraquat was addressed. This relationship is difficult to test directly, because, overexpression of PINK1 appears to accelerate the kinetics of Parkin recruitment to mitochondria (FIG. 12A-D), and so a kinase-deficient version of PINK1 (PINK1 KD) was used as a reporter for wild type PINK1 accumulation. PINK1 KD expression was regulated by mitochondrial voltage similarly to wild type PINK1 (FIG. 7D); but unlike wild type PINK1, PINK1 KD does not enhance Parkin recruitment when overexpressed (FIG. 12B). After treatment with paraquat overnight, PINK1 KD accumulated selectively on depolarized mitochondria, in a pattern similar to wild type PINK1 (FIG. 6I). In addition, a substantial subset of the mitochondria that accumulated PINK1 KD also recruited Parkin (FIG. 6I). While Parkin and PINK1 KD co-localize in paraquat treated cells (average pearson coefficient=0.45±0.13), PINK1 KD does not co-localize with MTR (average pearson coefficient=0.22±0.13; p-value=0.002 for PINK1 KD/Parkin vs. PINKKD/MTR) (FIG. 6J).

Considered together, these results demonstrate that PINK1 selectively accumulated on dysfunctional mitochondria with low membrane potentials.

### PINK1 Cleavage was Inhibited by Loss of Membrane Potential Leading to its Accumulation

Regulation of PINK1 expression at the level of transcription or translation would likely not be selective for a subpopulation of mitochondria. It was assessed whether increased PINK1 expression on damaged mitochondria is achieved by the selective removal of PINK1 from functional mitochondria. Full length PINK1 (˜63 kDa), which is anchored in the mitochondrial membrane, was proteolytically cleaved. into a ˜52 kDa cytosolic fragment that can be degraded by the proteasome. To test whether PINK1 accumulation following CCCP treatment is due to inhibition of its proteolytic cleavage, the effect of CCCP washout on PINK1 cleavage was assessed. HeLa cells were treated with vehicle (DMSO) or CCCP for 3 hours after which CCCP was either washed out or left in for an additional 30 minutes.

Cycloheximide was either added or left out during the final hour of treatment to control for de novo PINK1 synthesis during the washout period. Following PINK1 accumulation in the continuous presence of CCCP for 3 hours, the addition of cycloheximide for 30 minutes has little effect on the abundance of full length PINK1, suggesting that once it has accumulated, the ˜63 kDa PINK1 is relatively stable on depolarized mitochondria (FIG. 6F, lanes 4 vs. lane 6). However, within thirty minutes of CCCP washout, ˜63 kDa PINK1 abundance falls dramatically, consistent with its being cleaved by polarized mitochondria and maintained at low abundance on polarized, undamaged mitochondria (FIG. 6F, lanes 4-7 vs. lanes 8-11). The residual full length PINK1 seen following CCCP washout largely represents PINK1 that had accumulated during the 3 hr CCCP treatment, as the addition of cycloheximide prior to washout has little effect on its level (FIG. 6F, lane 8 vs. lane 10).

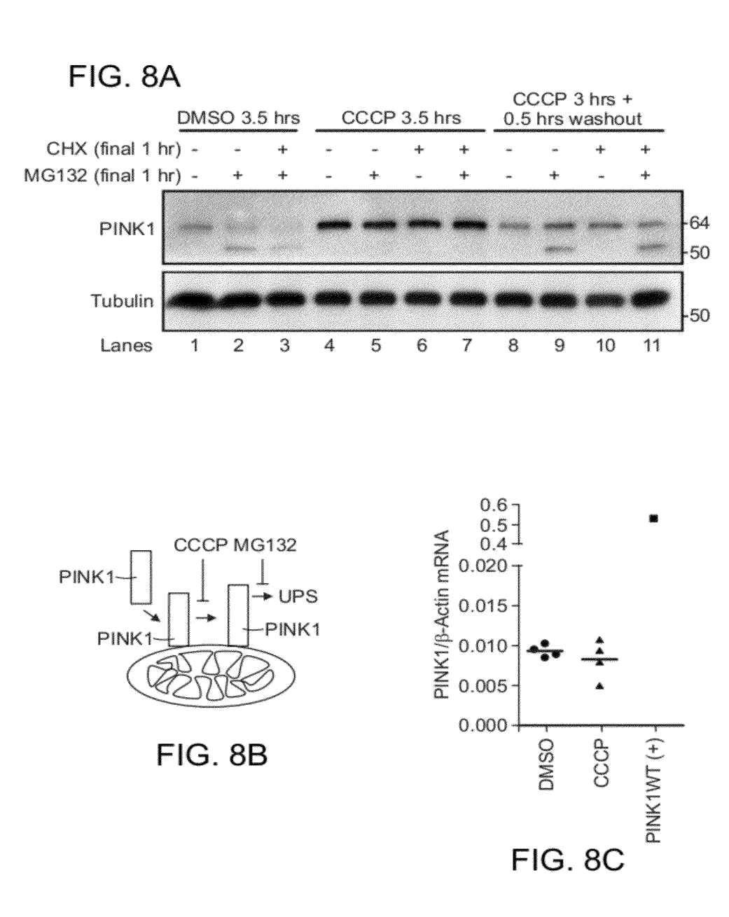

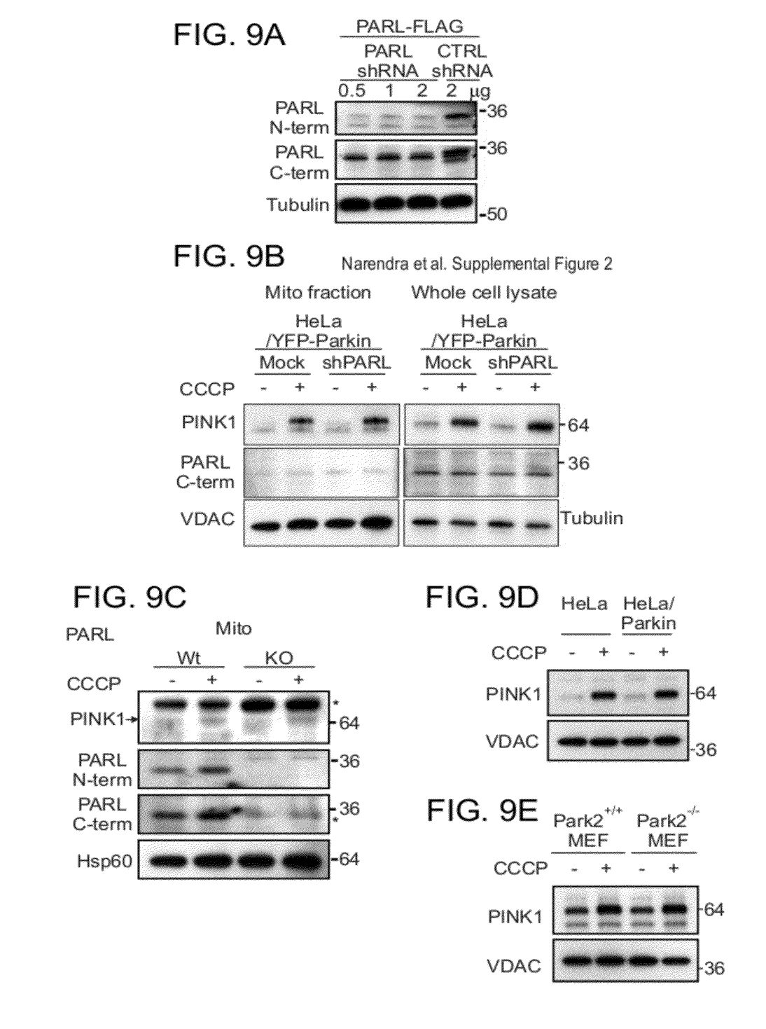

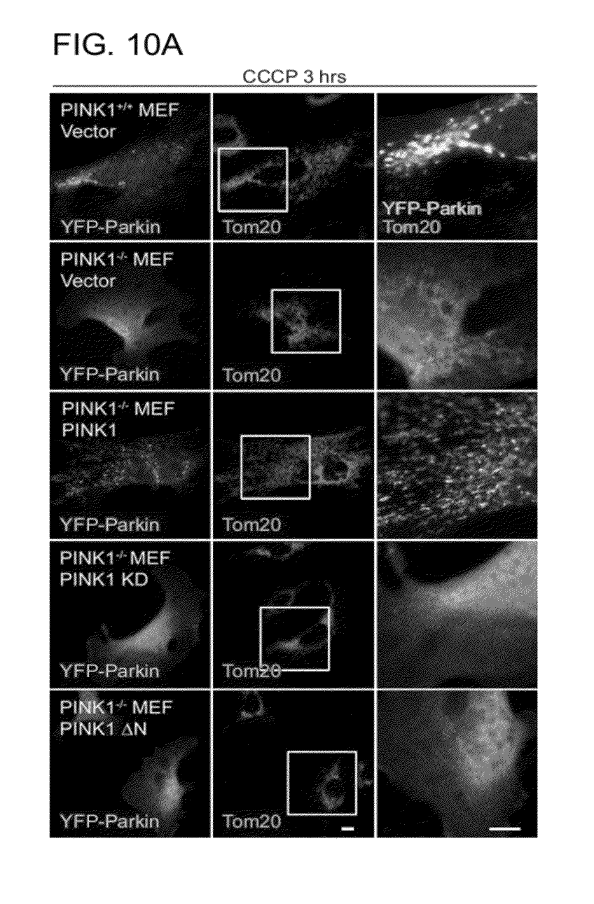

To further assess the stability of PINK1 under depolarizing conditions, the same set of experiments was performed in the presence of MG132, an inhibitor of proteasomal degradation. When MG132 was added during the final hour of treatment in HeLa cells treated with vehicle, a ˜52 kDa band appears, consistent with the cleavage product of full length Parkin described in previous reports (FIG. 6F, lane 1 vs. lane 2). The accumulation of this short form of PINK1 following treatment with MG132 suggests that it is unstable under basal conditions, as has been observed previously (FIG. 6F, lane 1 vs. lane 2). Interestingly, levels of the short form of PINK1 in the presence of MG132 decrease following depolarization with CCCP for 3.5 hours, as levels of full length PINK1 rise (FIG. 6F, lane 2 vs. lane 5); but increase following CCCP washout, as levels of full length PINK1 fall (FIG. 6F, lane 2 vs. lane 9). This pattern indicates that the cleavage of full length PINK1 into the unstable short form is blocked by mitochondrial depolarization and reinstated upon CCCP washout. Taken together, these results support a two-step model for the processing of PINK1: first, full length PINK1 is cleaved into the ˜52 kDa short form in a voltage-dependent, proteasome-independent manner, and, second, the short form of PINK1 is rapidly degraded by the proteasome (FIG. 8B). The voltage-dependent processing of PINK1 maintains low levels of PINK1 on healthy polarized mitochondria, but allows for the rapid accumulation of PINK1 on depolarized mitochondria.

Without wishing to be bound by theory, it is likely that the increased expression of PINK1 is due at least in part to inhibition of PINK1 cleavage. Nevertheless, it is possible that increased transcription of PINK1 following depolarization might also be contributing to the increase in PINK1 abundance. To assess whether PINK1 transcription is also regulated by membrane potential, quantitative RT-PCR of PINK1 levels in HeLa cells treated with DMSO or CCCP for 1 hr was performed. While exogenous expression caused a significant increase in PINK1 transcription relative to untransfected HeLa cells, PINK1 transcription did not significantly increase following depolarization with CCCP (p=0.4499). These data confirm that the increase in PINK1 expression following depolarization was not driven by an increase PINK1 transcription (FIG. 8C).

Finally, to test the localization of accumulated PINK1 on depolarized mitochondria, a protease protection assay was performed using an antibody raised against PINK1's kinase domain. Consistent with results from a recent study of ectopically expressed PINK1's topology, the kinase domain of endogenous PINK1 faced the cytosol following depolarization (FIG. 7E).

### PINK1 Accumulation on Depolarized Mitochondria is Independent of the Protease PARL

The protease responsible for PINK1 cleavage in mammalian cells is unknown, but in Drosophila cells the intramembrane serine protease, Rhomboid-7, appears to be required for PINK1 cleavage. To examine whether the mammalian orthologue of Rhomboid-7, PARL, is responsible for PINK1 cleavage in mammalian cells, the question of whether PINK1-V5 accumulates in HeLa cells transfected with PARL shRNA and treated with CCCP was explored. While endogenous PARL could not be detected in HeLa cells, PARL shRNA inhibited expression of overexpressed PARL (FIG. 9A and 9B). Knockdown of PARL did not appreciably change basal levels of endogenous PINK1 or inhibit the depolarization-induced accumulation of endogenous PINK1 in HeLa cells (FIG. 9B). Likewise, PINK1-V5 levels were similar in PARL^{-/-} and PARL^{+/+} MEFs, under basal conditions and following depolarization with CCCP (FIG. 9C). Together these results suggest that PARL is dispensable for PINK1 cleavage.

### PINK1 Accumulation on Depolarized Mitochondria was Independent of Parkin Expression

Previous studies in Drosophila and mammalian cells indicate that PINK1 functions genetically upstream of Parkin, although the molecular mechanism of this genetic interaction remains unexplained. To test whether PINK1 accumulation on mitochondria is upstream of Parkin recruitment to depolarized mitochondria, the dependence of PINK1 accumulation on Parkin expression was assessed. Endogenous PINK1 accumulated similarly in HeLa cells, which display little or no endogenous Parkin expression, and HeLa cells stably expressing YFP-Parkin (FIG. 9D). Consistent with these findings, exogenous PINK1-myc accumulated similarly in immortalized Parkin−/− and Parkin+/+MEFs (FIG. 9E). Together these results show that PINK1 accumulation is upstream of Parkin recruitment to depolarized mitochondria and independent of Parkin expression.

### PINK1 Expression was Required for Parkin Recruitment to Depolarized Mitochondria and Parkin-Induced Mitophagy

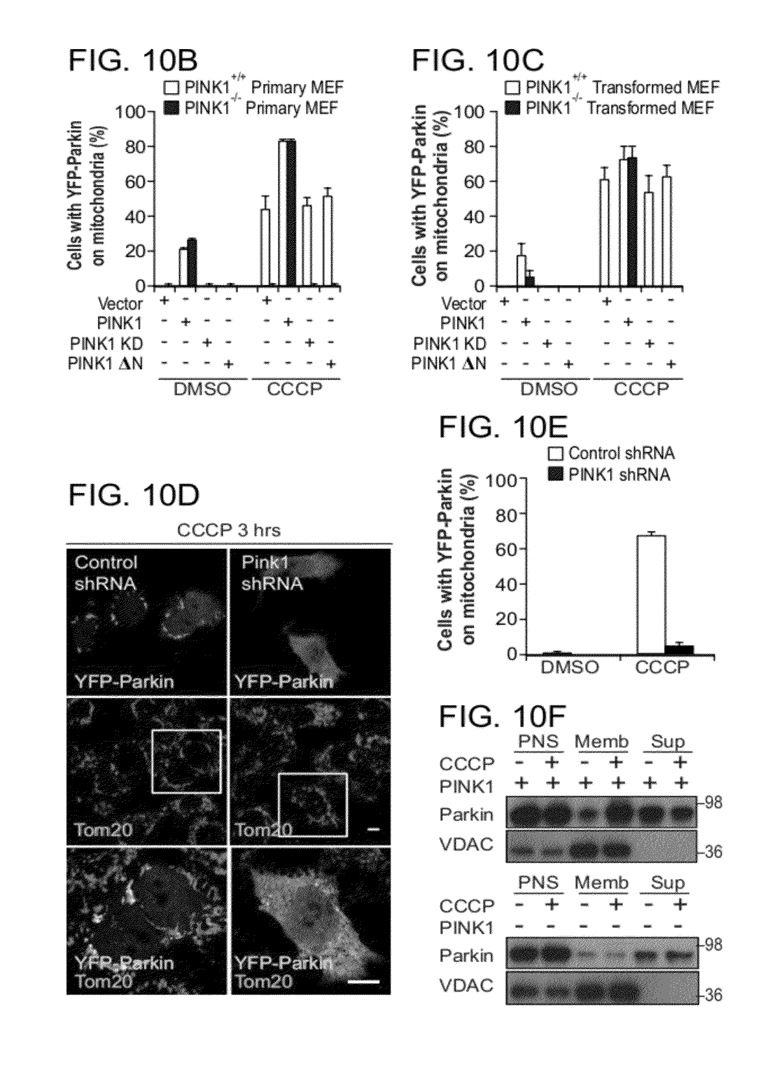

Next, the question of whether Parkin recruitment to depolarized mitochondria is dependent on PINK1 expression was addressed. Although YFP-Parkin was recruited to mitochondria in 43.3±8.1% (mean±s.d.) of PINK1+/+ primary MEFs after 3 hours exposure to 20 μM CCCP, it was not detectably recruited to mitochondria in PINK1−/− MEFs, as assessed by confocal microscopy (FIGS. 10A and 10B). YFP-Parkin recruitment at 24 hours following CCCP in PINK1−/−MEFs was also not detected, suggesting that little or no recruitment of YFP-Parkin to depolarized mitochondria occurs in the absence of PINK1. YFP-Parkin recruitment could be reconstituted in PINK1−/− MEFs by expression of wildtype PINK1, but not by PINK1 ΔN lacking its mitochondrial targeting N-terminus (1-155) suggesting that mitochondrial targeting of PINK1 is required for Parkin recruitment to mitochondria (FIG. 10A and 10B). A kinase-deficient (KD) version of PINK1 (Beilina et al., Proc Natl Acad Sci USA 102: 5703-5708, 2005) also failed to reconstitute Parkin recruitment to mitochondria.

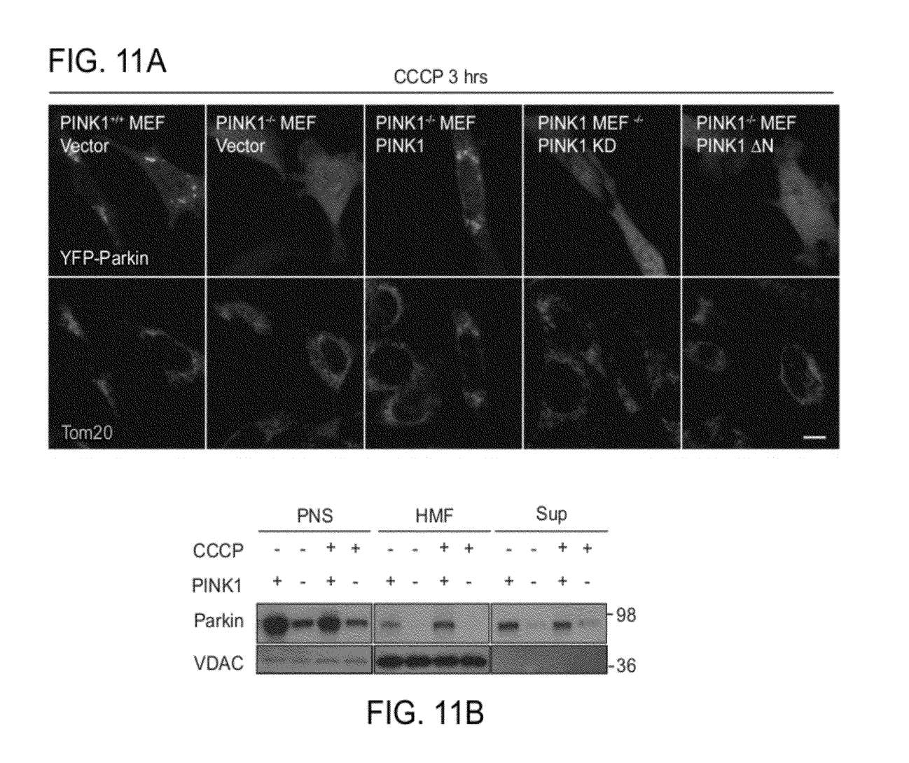

The dependence of Parkin recruitment on PINK1 in a SV40 transformed MEF cell line, which was derived from an independently generated PINK1−/− mouse (Xiong et al., J Clin Invest 119: 650-660, 2009) was tested (FIG. 10C and FIG. 11A). Similar to the primary PINK1−/− MEFs, no recruitment is seen in the transformed PINK1−/− cells, while Parkin is recruited to mitochondria in 60.7±7.7% of PINK1+/+ cells upon CCCP treatment. Likewise, Parkin recruitment in the transformed PINK1−/− cells is reconstituted following exogenous expression of PINK1 (72.8±7.7% vs. 0.0±0.0%, p-value<0.001) but not PINK1 ΔN or PINK1 KD.

Finally, the dependence of Parkin recruitment in a human neuroblastoma cell line (M17) was tested. In M17 cells stably transduced with PINK1 shRNA, YFP-Parkin translocated to mitochondria in 4.7±1.2% of CCCP treated cells, whereas 67.3±3.1% of control shRNA M17 cells displayed mitochondrial YFP-Parkin after treatment with 10 μM CCCP for 3 hours (p-value<0.001) (FIG. 10D, 10E). Vehicle treatment failed to induce YFP-Parkin translocation to mitochondria in both cell lines. The necessity of PINK1 expression for Parkin recruitment to membranes was also examined in the M17 cell line by immunoblotting. In control shRNA cells, YFP-Parkin levels increase in the mitochondria-rich membrane fraction and decrease in the supernatant following treatment with CCCP, consistent with Parkin translocation to mitochondria (FIG. 10F upper panel and FIG. 11B). YFP-Parkin was expressed less in the PINK1 shRNA cells compared to control shRNA cells, possibly because the transfection efficiency is lower in these cells and/or because Parkin is less stable in the absence of PINK1. Nonetheless, no Parkin increase was observed in the membrane fraction either under equal loading conditions or when loading was adjusted so that total Parkin was approximately equal in the two cell populations, further indicating that Parkin is not recruited to uncoupled mitochondria in the absence of PINK1 (FIG. 10F lower panel and FIG. 11B).

Ectopic Parkin can induce the autophagy of depolarized mitochondria. To test whether PINK1 is necessary for Parkin-induced mitophagy, primary PINK1 and PINK1^{+/+} MEFs transiently expressing YFP-Parkin with 20 μM CCCP were treated for 24 hours (FIGS. 12A and 12B). While no mitochondria can be detected in 66.1±16.8% of PINK1^{+/+} MEFs, all PINK^{-/-} MEFs retain their mitochondria. Parkin-dependent mitophagy is reconstituted by exogenous PINK1 expression in the PINK^{-/-} MEFs with 65.5±5.0% of reconstituted PINK^{-/-} cells displaying undetectable mitochondria following CCCP treatment.

Parkin-induced mitophagy was also dependent on PINK1 expression in the M17 human neuroblastoma cell line. Whereas in 27.1±8.6% of control shRNA M17 cells displayed complete loss of mitochondria after 24 hours, less than 5% of cells lost mitochondria in the PINK1 shRNA cells (FIGS. 12C and 12D). These results suggest that PINK1 is necessary for the mitophagy of depolarized mitochondria following overexpression of Parkin.

To test whether PINK1 expression affects mitochondrial turnover in the presence of endogenous levels of Parkin, the control shRNA and PINK1 shRNA M17 cells (which express moderate levels of Parkin) were treated with DMSO or CCCP for 24 hours and measured their relative mitochondrial mass by Mitotracker Green (MTG) staining and flow cytometry. MTG, a sensitive measure of mitochondrial mass, stains mitochondrial lipid in a membrane potential independent manner and has been used to measure mitochondrial mass of depolarized mitochondria previously (Hristova et al., J Biol Chem. In press; Whitworth et al., Dis Model Mech 1: 168-174, 2008). Control shRNA M17 cells exhibited a decrease in mitochondrial mass (CCCP vs. DMSO, −22.4±12.6%) following CCCP treatment, while PINK1 shRNA M17 cells exhibited an increase in mitochondrial mass (CCCP vs. DMSO, 43.5±20.0%) following depolarization (p-value=0.008 for change in mitochondrial mass control shRNA vs. PINK shRNA) (FIG. 1E). These results are consistent with endogenous PINK1 promoting mitochondrial degradation in the context of continued (or increased) mitochondrial biogenesis. To more directly assay mitochondrial turnover in control and PINK1 shRNA M17 cells, the cells were pulsed with MTG and loss of MTG intensity was tracked at 0, 16, and 24 hours in the presence of CCCP. Consistent with the hypothesis that endogenous PINK1 promotes the degradation of depolarized mitochondria, Mitotracker Green intensity decreased more slowly in PINK1 shRNA cells when compared with control shRNA cells, treated with CCCP (0.58±0.07 vs. 0.33±0.07 relative MTG intensity at 24 hours) (FIG. 12F). These data indicate that PINK1 promotes mitophagy in the context of endogenous levels of Parkin. Additionally, these results indicate that the selective turnover of dysfunctional mitochondria may be balanced by the biogenesis of new mitochondria, allowing exchange of damaged, dysfunctional mitochondria for healthy, functional mitochondria.

Consistent with genetic studies in Drosophila, these findings show that Parkin translocation to depolarized mitochondria and Parkin-induced mitophagy are downstream of PINK1 expression, while PINK1 accumulation in response to depolarization is upstream of Parkin recruitment.

### Expression of PINK1 on the Outer Mitochondrial Membrane is Sufficient for Parkin Recruitment and Mitophagy

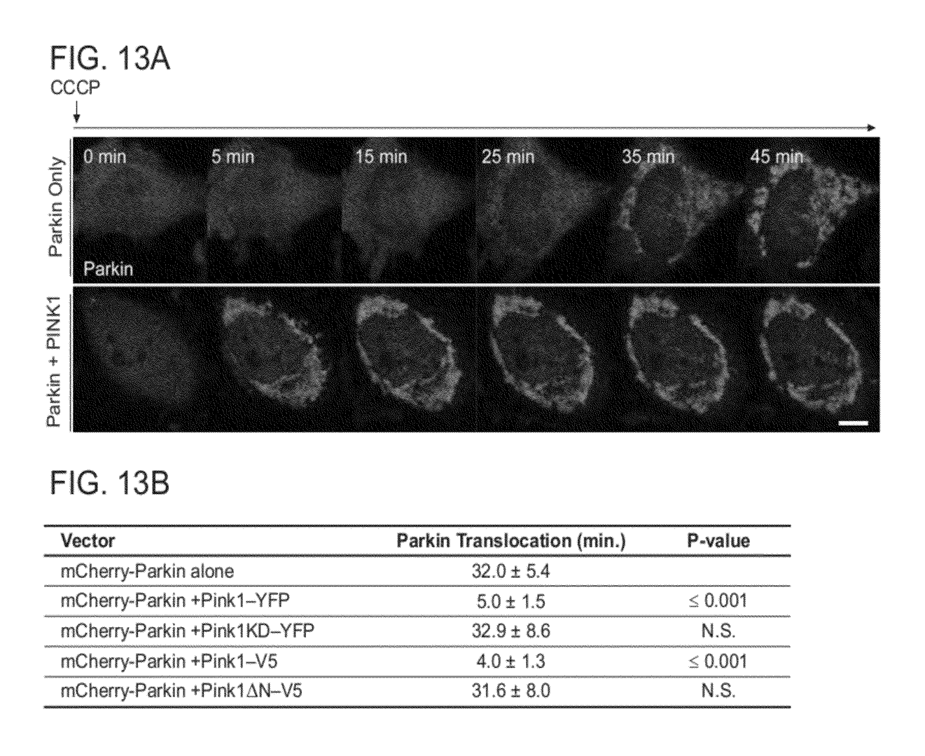

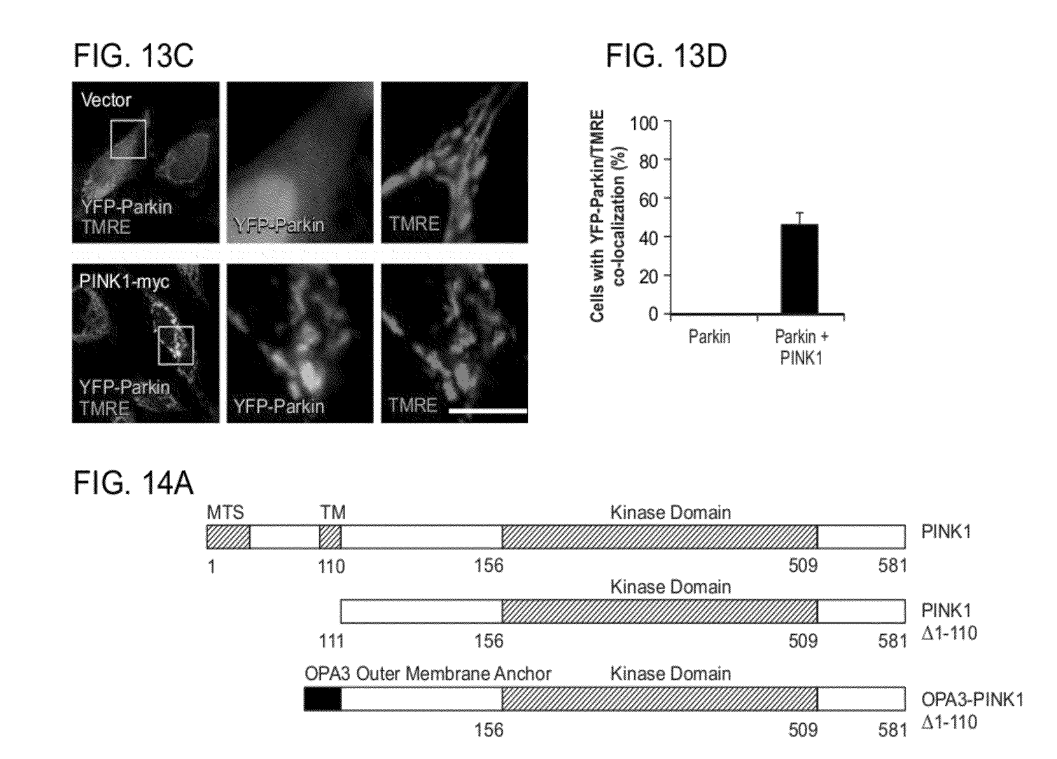

The expression of mitochondrial PINK1 is necessary for recruitment of Parkin to mitochondria. To determine whether PINK1 overexpression is sufficient for Parkin recruitment to mitochondria, live cell imaging was used. Moderate overexpression of PINK1 dramatically accelerated the kinetics of Parkin recruitment following depolarization with CCCP (time to translocation 5.0±1.5 minutes vs. 32.0±5.4 min, p-value<0.001) (FIGS. 13A and 13B;). Consistent with the necessity of PINK1's mitochondrial localization and kinase activity, exogenous expression of PINK1 KD or PINK1ΔN failed to accelerate the kinetics of Parkin recruitment (FIG. 12B). In cells with high expression of PINK1, Parkin is recruited to mitochondria even in the absence of CCCP (FIGS. 13C and D). YFP-Parkin co-localized with the potentiometric mitochondrial dye TMRE in 45.3±7.6% of cells co-expressing YFP-Parkin and PINK1 vs. 0±0% of cells expressing Parkin alone (p-value<0.001) (FIGS. 13C and D). Together these results demonstrate that the kinetics of Parkin recruitment is exquisitely sensitive to PINK1 levels in the cell. In addition, they indicate that increased PINK1 expression is sufficient for Parkin recruitment independent of membrane potential.

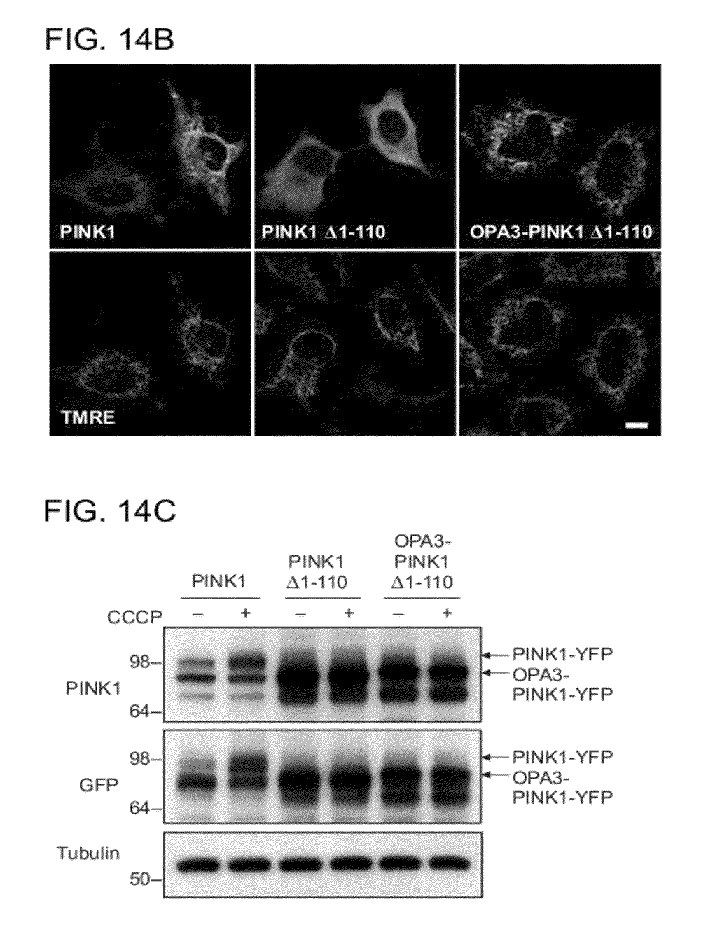

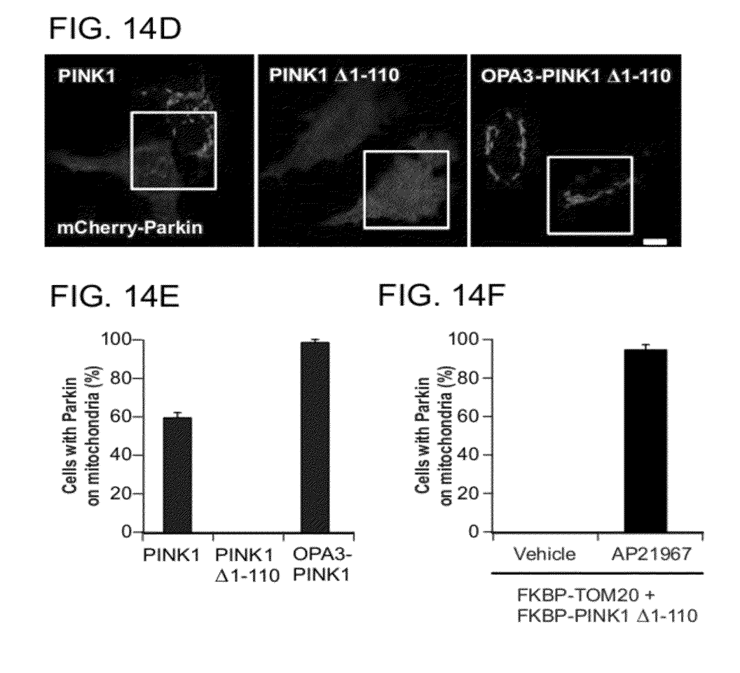

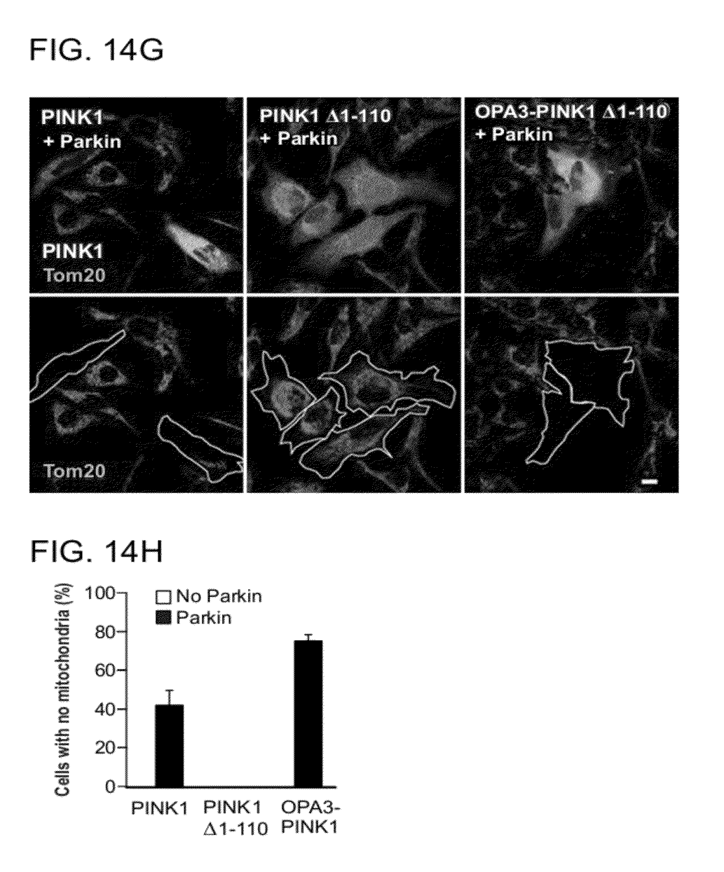

To test whether stable expression of PINK1 on the mitochondria is sufficient for Parkin recruitment, a fusion protein was constructed that would be predicted to lack PINK1's proteolytic cleavage site and therefore exhibit greater stability on mitochondria. Based on the ˜11 kDa difference between the full length form and cleaved form, the cleavage site likely lies before residue 110 (residues 1-110 have a predicted molecular weight of 11.54 kDa), and so residues 1-110 of PINK1 were replaced with the outer mitochondrial membrane anchor from OPA3 (1-30) (FIG. 14A). Removing the first 110 amino acids of PINK1 prevented targeting of PINK1 to mitochondria (FIG. 13B, middle panel); while the fusion of OPA3 (1-30) to PINK1 Δ1-110 restored mitochondrial targeting, likely to the outer mitochondrial membrane (FIG. 14B, right panel). As predicted by the proteolytic cleavage results (FIG. 8A), OPA3-PINK1 Δ1-110-YFP exhibited increased stability compared to PINK1-YFP (FIG. 14C). In addition, OPA3-PINK1-YFP levels did not respond to mitochondrial depolarization with CCCP, indicating that stabilization of PINK1 by depolarization depends on its first 110 amino acids. When co-expressed with mCherry-Parkin, PINK1-YFP recruits mCherry-Parkin to mitochondria in 57.9±1.8% of cells in the absence of CCCP; while PINK1 Δ1-110-YFP, which is not expressed on mitochondria, failed to recruit mCherry-Parkin in the absence of CCCP. However, OPA3-PINK1 Δ1-110-YFP, which does not display voltage dependent proteolysis, recruited mCherry-Parkin to mitochondria in 98±1.8% of cells in the absence of CCCP (FIGS. 14D and E). Together these data demonstrate that stable expression of PINK1 on mitochondria is sufficient for Parkin recruitment to mitochondria, regardless of membrane potential.

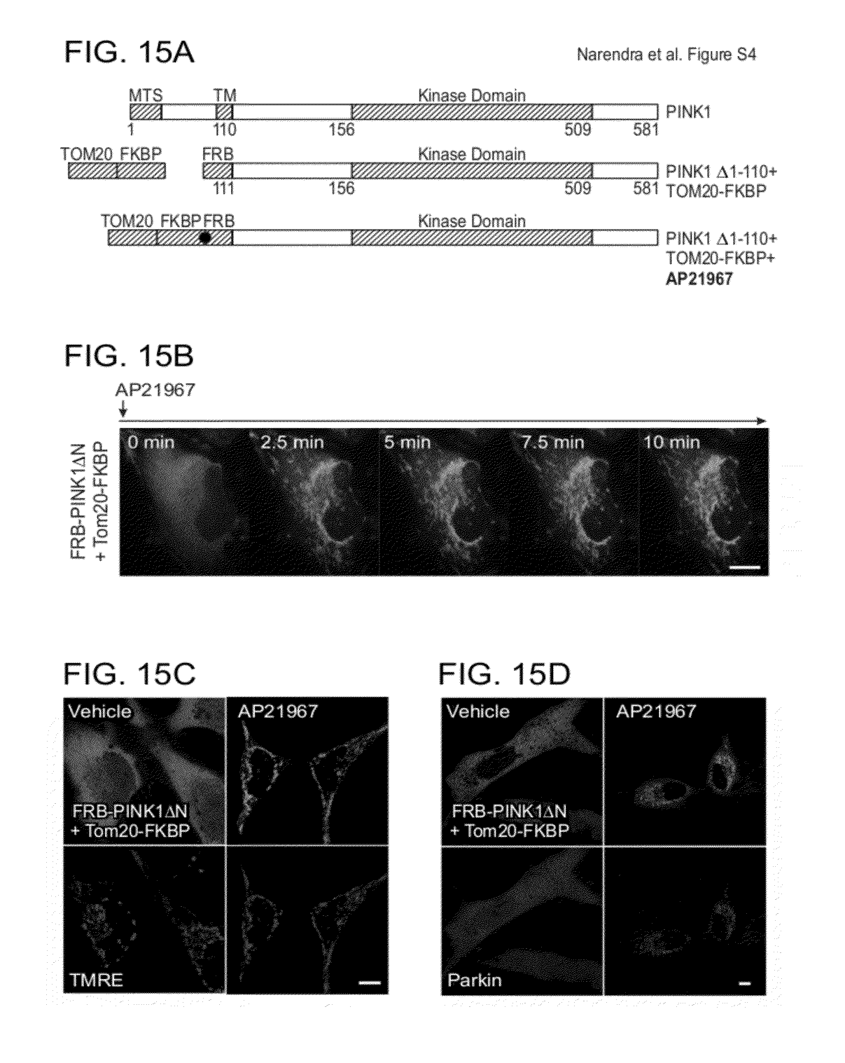

To verify that increased expression of PINK1 on the outer mitochondrial membrane is sufficient to induce Parkin recruitment, a regulated heterodimerization system was used, in which the modified FRB domain was fused to PINK1 Δ1-110-YFP and the FKBP domain was fused to the outer mitochondrial membrane anchor of TOM20 (residues 1 through 33) (FIG. 15A). In the presence of the rapamycin derivative AP21967, the FRB domain and the FKBP domain heterodimerize, but only if they are in the same compartment. Thus, FRB-PINK1Δ1-110-YFP should be recruited from the cytosol to mitochondria if the FKBP domain of TOM20-FKBP faces the cytosol but not if it faces the inner membrane space or the matrix. FRB-PINK1Δ1-110-YFP is in the cytosol in the absence of AP21967, but is quickly recruited to the outer mitochondrial membrane following the addition of AP21967 (FIGS. 15B and C). To assess whether PINK1 expression on the outer mitochondrial membrane is sufficient to recruit Parkin, mCherry-Parkin, FRB-PINK1Δ1-110-YFP, and TOM20-FKBP were co-transfected. In the absence of AP21967, mCherry-Parkin was in the cytosol, but following incubation AP21967 mCherry-Parkin was recruited to mitochondria in 96.7±4.1% of cells, in the absence of CCCP (FIG. 14F and FIG. 15D). Thus, increased expression of PINK1 on the outer mitochondrial membrane was sufficient to recruit Parkin to mitochondria.

Next, the question of whether Parkin recruitment following increased PINK1 expression on the mitochondria was sufficient to induce mitophagy in the absence of depolarization with CCCP was explored. Co-transfection of PINK1 and Parkin resulted in a substantial proportion of cells (42.1±7.3%) with no mitochondria after 96 hours. By contrast, co-transfection of cytosolic PINK1 Δ1-110-YFP with Parkin produced no cells lacking mitochondria after 96 hours. Fusing the outer membrane anchor of OPA3 to PINK1 Δ1-110-YFP, which results in stable expression of PINK1 on mitochondria, restored the ability of PINK1 and Parkin to induce mitophagy, with 76.4±2.2% of cells lacking mitochondria at 96 hours (FIGS. 14G and H). These data demonstrate that stable expression of PINK1 on the outer mitochondrial membrane is sufficient to induce mitophagy in the presence of Parkin, irrespective of membrane potential.

### PINK1 Accumulation Following Depolarization is Necessary for Parkin Recruitment to Mitochondria

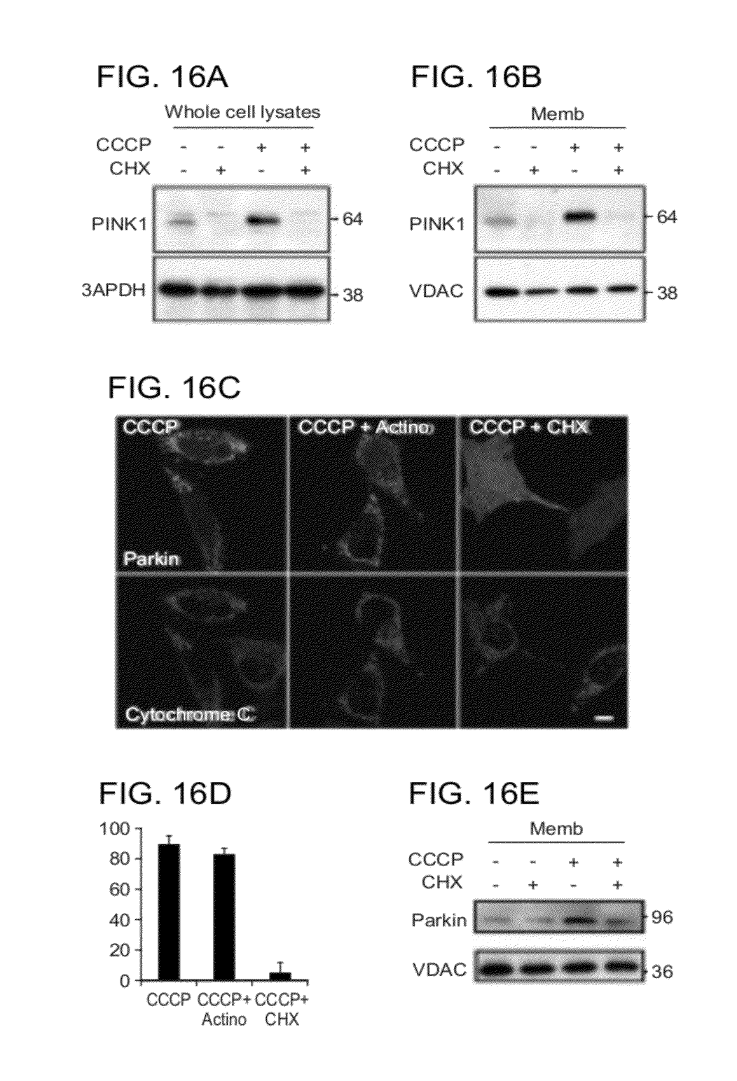

To test whether accumulation of endogenous PINK1 following depolarization is necessary for Parkin recruitment, HeLa cells were treated with CCCP alone (for 60 minutes) or with CCCP plus cycloheximide, a general inhibitor of protein synthesis (cycloheximide was added 30 minutes before CCCP and maintained throughout the 60 minute CCCP treatment). Treatment of HeLa cells for 90 minutes with cycloheximide blocked the depolarization-induced accumulation of endogenous PINK1 in whole cell lysates as well as in the mitochondrial-rich membrane fraction (FIGS. 16A and B). 90 minute treatment with cycloheximide, likewise, blocked Parkin recruitment to depolarized mitochondria by confocal microscopy (96.0±3.5% vs. 11.3±4.2%) (FIGS. 16C and D). By contrast, 90 minute treatment with actinomycin D, an inhibitor of transcription, had a modest effect on Parkin recruitment to uncoupled mitochondria by confocal microscopy (FIGS. 16C and D), suggesting that new transcription of PINK1 is not required for Parkin recruitment. This is consistent with the absence of PINK1 mRNA upregulation following uncoupling (FIG. 8C). Cycloheximide likewise blocked YFP-Parkin accumulation in the mitochondrial enriched heavy membrane fraction by immunoblotting (FIG. 16E). Based on these findings, it is likely that PINK1 accumulation and Parkin recruitment are causally related.

### Threonines 175 and 217 in Parkin May not be Involved in Parkin Recruitment to Mitochondria

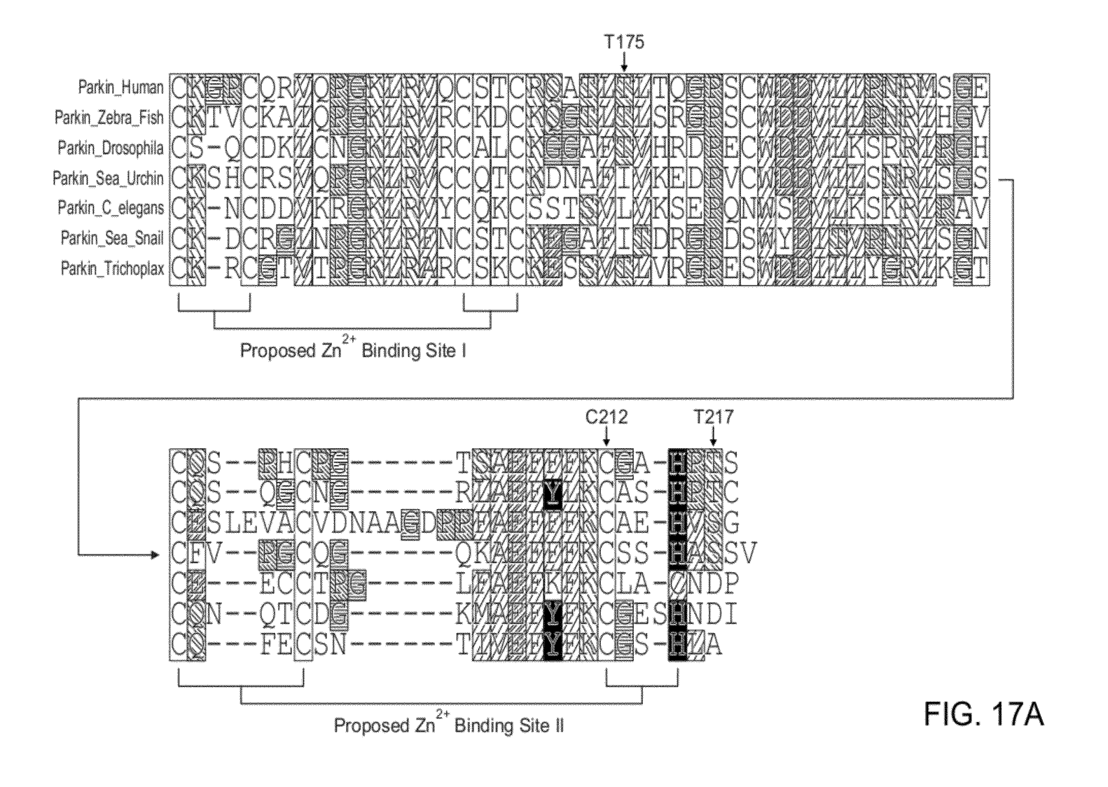

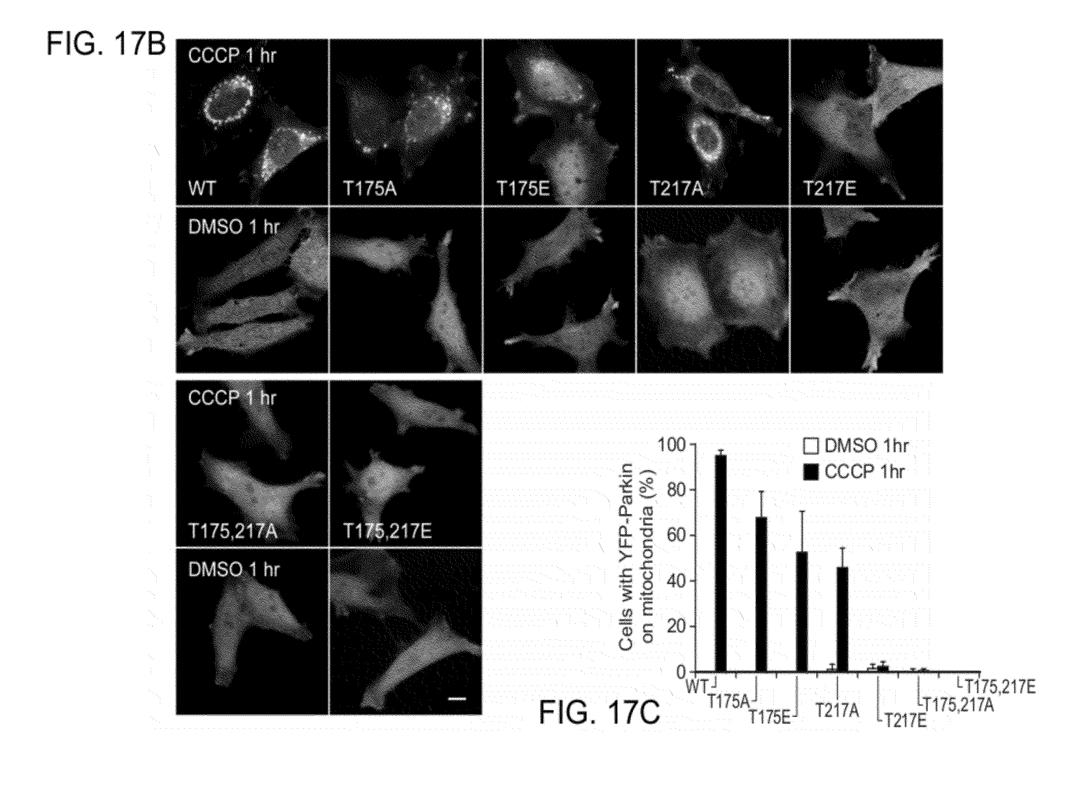

It has been proposed that PINK1 may induce mitochondrial recruitment of Parkin through phosphorylation of threonines 175 and 217 in a highly conserved region/domain of Parkin, which has been recently named RING0 (FIG. 17A) (Kitada et al. (2007) Proc Natl Acad Sci USA 104: 11441-11446). Although mutation of T175 and T217 to alanine blocked recruitment of Parkin to mitochondria, as was reported previously, the phosphomimetic mutants T175E, T217E, and T175, 217E did not translocate to mitochondria spontaneously. In addition, these phosphomimetic mutants appear to inhibit CCCP-induced recruitment of Parkin. While these findings do not rule out the possibility that phosphorylation of these sites by PINK1 or another kinase induces Parkin recruitment, it is likely that these threonines are more likely to play an important structural role (FIGS. 17B and C).

### Patient Mutations in PINK1 and Parkin Disrupt PINK1/Parkin Pathway at Distinct Steps

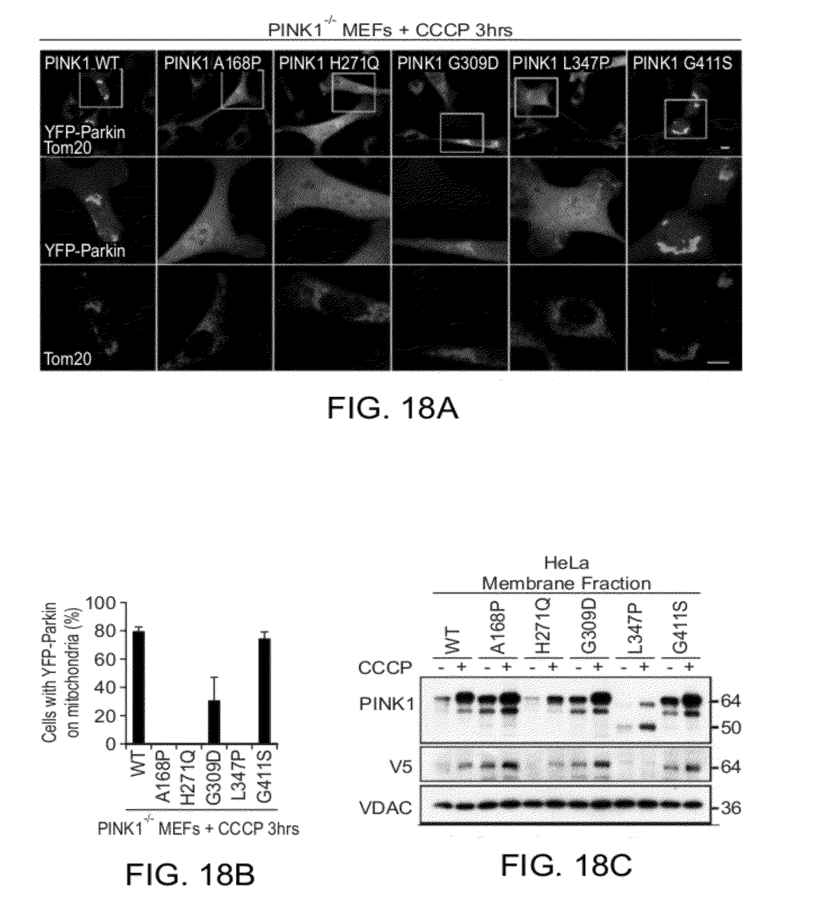

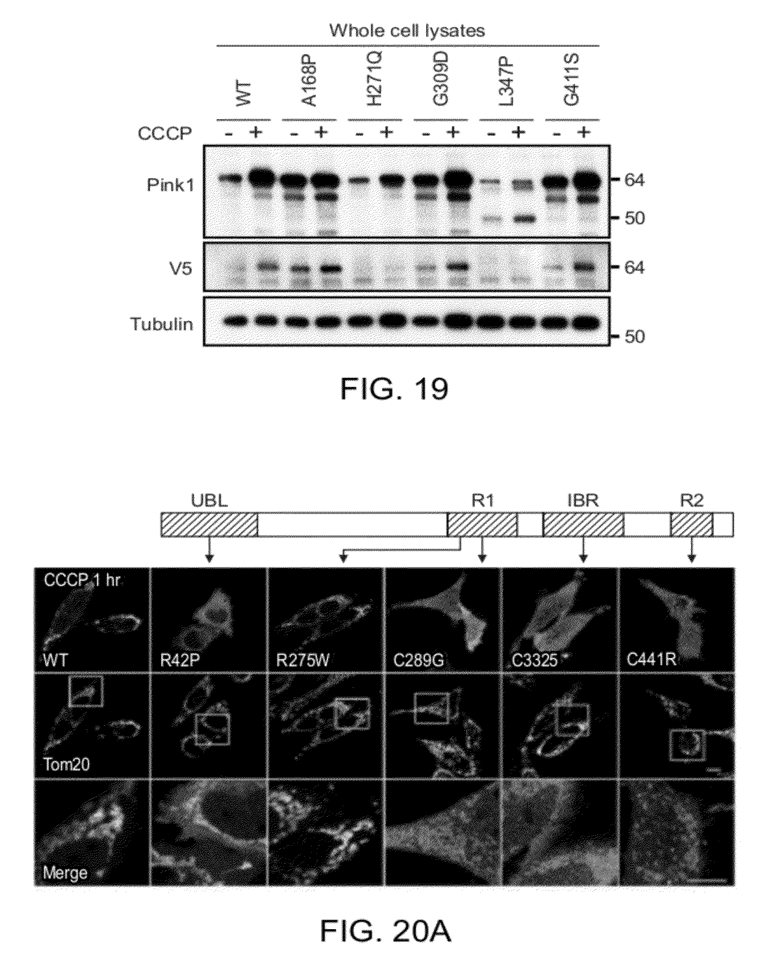

The ability of disease-causing mutations in PINK1 to reconstitute YFP-Parkin recruitment to mitochondria in PINK1−/− primary MEFs was assessed. Following exogenous PINK1 WT expression in PINK1−/− MEFs, YFP-Parkin was recruited to mitochondria in 78.6±3.9% of cells after 20 μM CCCP treatment for 3 hours (FIG. 18A and 18B). The L347P patient mutant of PINK1 was unstable (FIG. 13C and FIG. 18A), as was reported previously (Beilina et al., Proc Natl Acad Sci USA 102: 5703-5708, 2005), and that L347P failed to reconstitute YFP Parkin recruitment to depolarized mitochondria (FIGS. 18A and 18B). Of the patient mutations that exhibited stable expression, A168P and H271Q also failed to reconstitute YFP-Parkin recruitment at 3 hours, while G309D, only partially reconstituted YFP-Parkin recruitment (30.7±16.7%) (FIGS. 18A and 18B). The polymorphism G411S, which to date has only been found in cases heterozygous for the mutation (Abou-Sleiman P M, Muqit M M, McDonald N Q, Yang Y X, Gandhi S, et al. (2006) Ann Neurol 60: 414-419), reconstituted YFP-Parkin recruitment to a similar extent as wildtype PINK1 (74.2±5.4%), suggesting that PINK1 containing this polymorphism may be functional in the PINK1/Parkin pathway (FIGS. 17A and 17B). This is consistent with the idea that G411S may represent a functional polymorphism and may not be a true disease-causing mutation. Protein levels of all PINK1 mutants accumulated upon exposure of cells to CCCP (FIG. 18C and FIG. 19A).

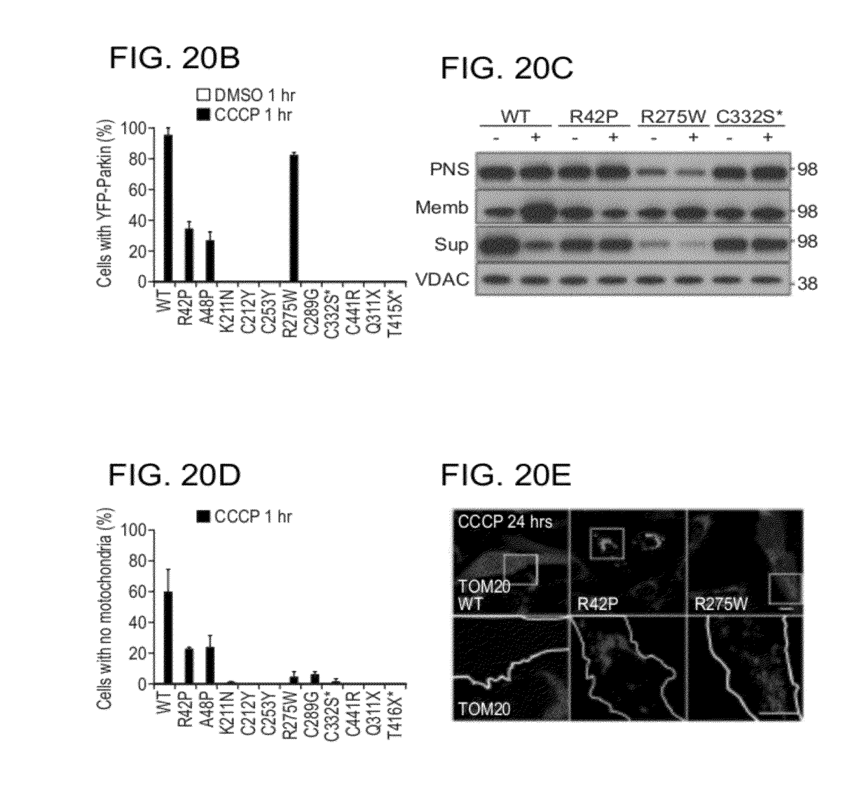

Next, patient mutations in Parkin were tested to see if they would affect Parkin recruitment to mitochondria and/or Parkin-induced mitophagy. Parkin has an N-terminal ubiquitin-like domain (UBL) and a C-terminal RING-between-RING (RBR) superdomain, which consists of three atypical RING domains (FIG. 20A). The fold of the N-terminal RING1 most closely resembles that of traditional RING domains, such as that of c-CBL, while the In-Between-RING (IBR) and the C-terminal RING2 likely have unique folds. The RBR domain is responsible for Parkin's ubiquitin ligase activity, while its UBL domain is thought to mediate interactions between Parkin and proteins with ubiquitin-binding domains (UBDs).

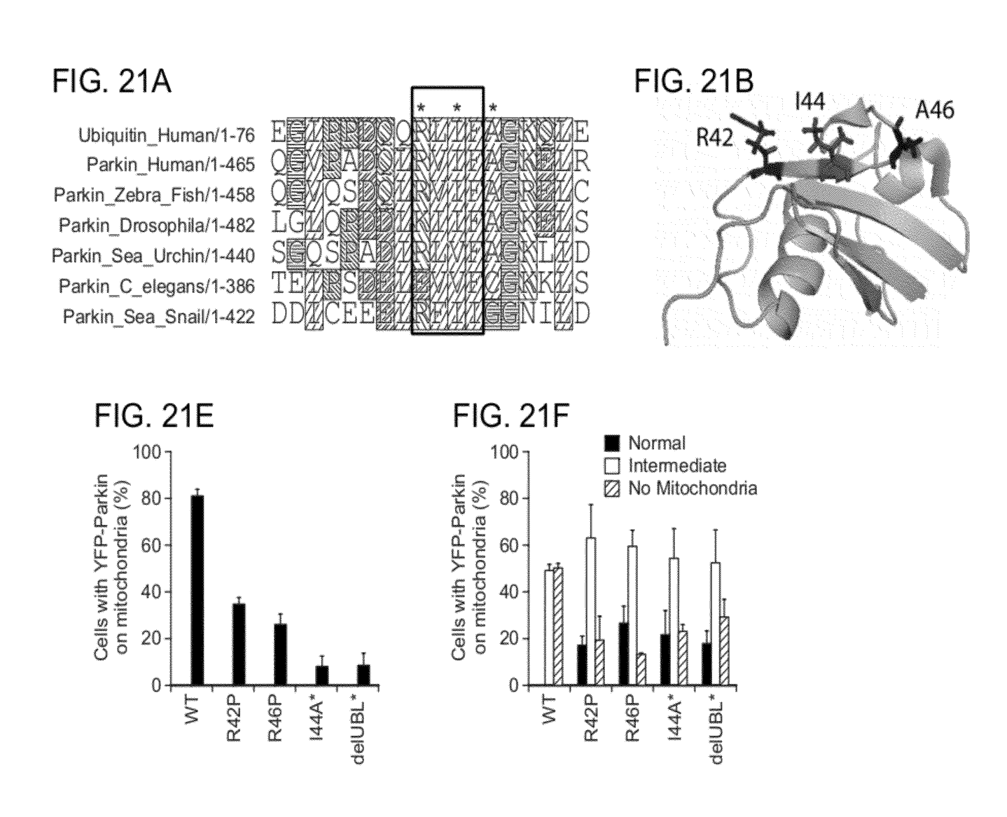

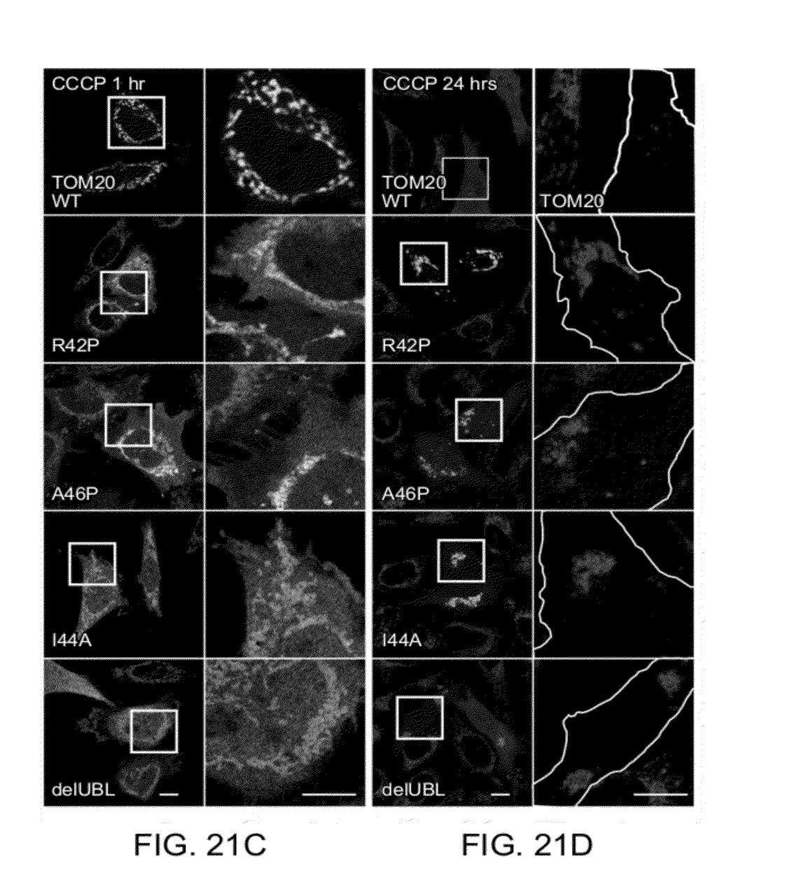

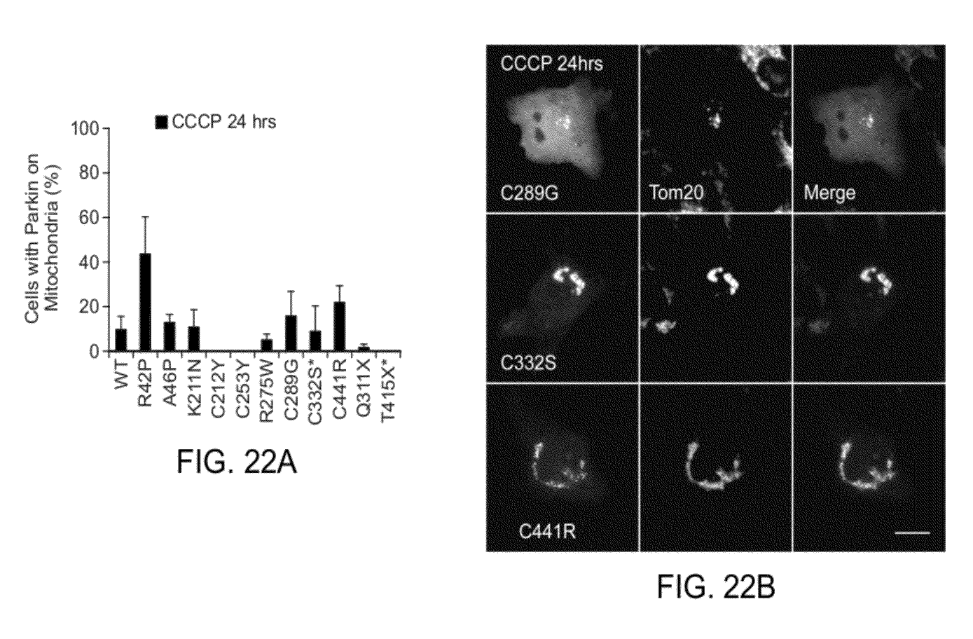

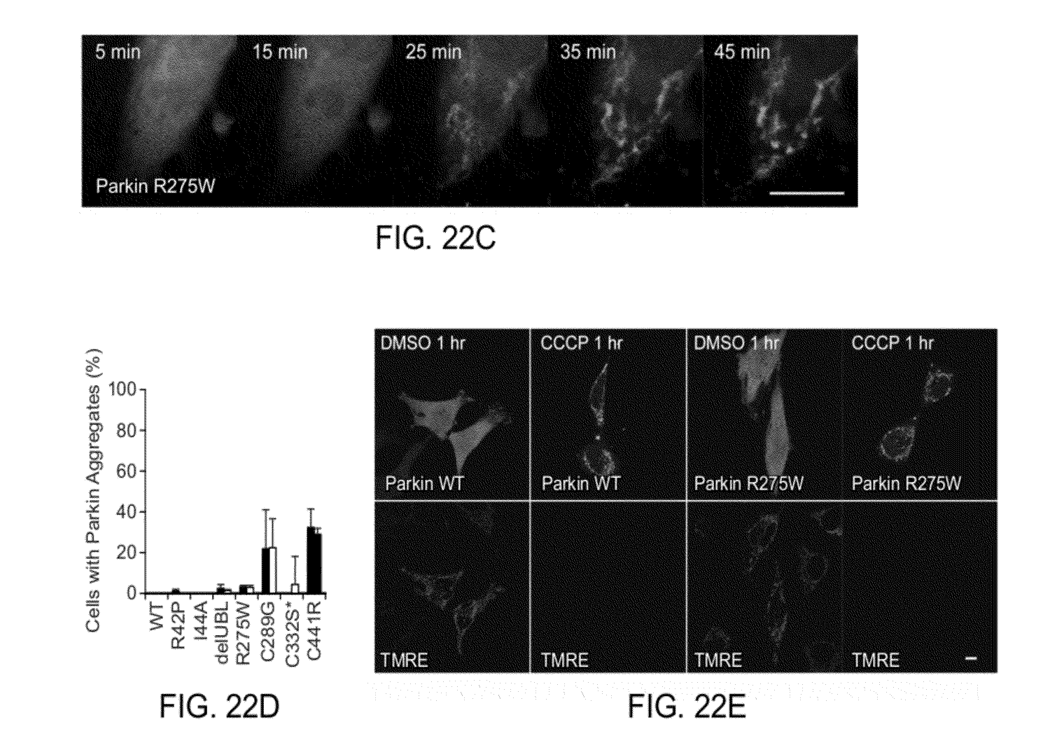

Wildtype YFP-Parkin is recruited to mitochondria in the majority of HeLa cells (94.7±5.8%) by confocal microscopy, following treatment with 10 μM CCCP for 1 hr (FIG. 20A). Pathogenic mutations in the UBL domain (R42P and R46P), deletion of the UBL, or mutation of a key residue (I44A) in the interaction of UBLs with UBDs, all cause a moderate deficit in Parkin recruitment to depolarized mitochondria (34±5.3% and 26.5±6.6% for R42P and R46P, respectively) (FIGS. 16A and B and FIG. 21A-21E). Mutations in conserved cysteines of the RING domains (the patient mutations C253Y, C289G, and C441R and the engineered mutation C332S) completely disrupt recruitment at 1 hr of CCCP treatment, as do mutations (patient mutation Q311X and engineered mutation T415X) that result in loss of RING2 (FIGS. 21A and 21B). Mutations K211N and C212Y, which lie within a highly conserved region of Parkin that is likely a novel RING-like domain (FIG. 20), similarly blocked the recruitment of Parkin to mitochondria (FIG. 18B), consistent with the importance of this region for Parkin's activity. Mitochondrial recruitment was seen for several of the conserved cysteine RING mutants (C289G, C332S, and C441R) after 24 hours of CCCP exposure, suggesting that recruitment is not completely disrupted with these mutations (FIGS. 22A and B). Interestingly, the R275W mutation in RING1 exhibited only a mild deficit in recruitment (81.7±2.1%) (FIGS. 16A and 16B). The recruitment of YFP-Parkin R275W was verified in a live cell imaging experiment (FIG. 22C). Although under control conditions some mutants formed visible aggregates (FIG. 22D), no mutant, including R275W, colocalized with mitochondria (FIG. 20B and FIG. 22E).

Next, recruitment of Parkin mutants to depolarized mitochondria was assessed by immunoblotting. Some background YFP-Parkin signal in the membrane fraction was observed under control conditions. Following treatment with CCCP for 1 hr, levels of wildtype Parkin increase in the mitochondria-rich membrane fraction and decrease in the supernatant (FIG. 20C). Although expression of Parkin R275W was moderately less than wildtype, it also increases localization in the membrane fraction and decreases in the supernatant upon CCCP treatment, consistent with the mitochondrial translocation seen for this mutation by confocal microscopy (FIG. 20C). No membrane translocation was detectable by Western blotting for either R42P or C332S, however, suggesting that translocation of these mutants is substantially lower than wildtype and consistent with the deficit in mitochondrial translocation seen by confocal microscopy (FIG. 20C).

The ability of Parkin mutants to induce mitophagy was assessed. Expression of wildtype Parkin in HeLa cells that do not detectably express endogenous Parkin completely eliminates mitochondria in greater than half of the cells (59.0±15.1%) following treatment with CCCP for 24 hours (FIGS. 20D and E). Mutations in the UBL of Parkin exhibit a moderate loss in mitophagy activity (22.0±2.0% and 23.1±8.4% of cells exhibited no mitochondria for R42P and R46P, respectively); while mutations in the conserved cysteines of the RBR or truncations that resulted in loss of RING2 exhibited a severe mitophagy deficit (0±0% to 5.3±2.3%, depending on the mutation) (FIGS. 20D and E). In addition, patient mutations R211N and C212Y caused a similar deficit in mitophagy, supporting the notion that this may be an atypical RING domain similar to RING1, IBR, and RING2 (FIGS. 19D and 19E). Interestingly, the R275W mutation in RING1 also exhibited a severe deficit in mitophagy (4.0±4%) (FIGS. 20D and E) even though it appears to largely retain its ability to translocate to uncoupled mitochondria (FIGS. 20A and 20B). This pattern of findings suggests that recruitment of Parkin to mitochondria and its induction of mitophagy are dissociable events.

The Parkinson's-linked E3 ubiquitin ligase, Parkin, is selectively recruited to dysfunctional mitochondria with low membrane potential to promote their autophagic degradation, suggesting that a deficiency of mitochondrial quality control is a potential mechanism for the observed mitochondrial dysfunction in Parkin knockout Drosophila and mice. How Parkin is able to distinguish damaged, depolarized mitochondria from healthy, polarized mitochondria, however, was unknown.

As reported herein, PINK1 selectively accumulated on depolarized mitochondria that have sustained damage. This selective accumulation is achieved by a novel mechanism, in which PINK1 is constitutively synthesized and imported into all mitochondria, but cleaved from healthy mitochondria by voltage-sensitive proteolysis (FIG. 22). On damaged mitochondria that have lost their membrane potential, however, PINK1 cleavage is inhibited leading to high PINK1 expression on the dysfunctional mitochondria. Expression of mitochondrial PINK1 is required for the recruitment of Parkin to the dysfunctional mitochondria and for their selective elimination by Parkin. In addition, increased expression of PINK1 on the outer mitochondrial membrane is sufficient for Parkin recruitment and Parkin-induced mitophagy, suggesting that loss of membrane potential activates Parkin recruitment primarily through the upregulation of mitochondrial PINK1.

As reported herein, the kinase PINK1 is constitutively down-regulated posttranslationally in a manner that depends on normal mitochondrial membrane polarization. Rapid turnover of PINK1 on polarized mitochondria proteolytically generates a ˜52 kDa fragment that is quickly eliminated by the proteasome. Pharmacologic uncoupling of mitochondria leads to a dramatic upregulation of PINK1 expression. When a subset of mitochondria within one cell is uncoupled, PINK1 accumulates selectively on the dysfunctional organelles. Both PINK1 expression and the translationally-mediated accumulation of PINK1 are required for Parkin recruitment to depolarized mitochondria. As ectopic expression of PINK1 targeted to mitochondria (OPA3-PINK1 Δ1-110) is sufficient to recruit Parkin to polarized functional mitochondria, the role of membrane potential loss in Parkin translocation appears to be solely through PINK1 stabilization. Consistent with this model, recruitment of Parkin to depolarized mitochondria requires PINK1 mitochondrial targeting. PINK1 expression was also strictly required for Parkin-induced autophagy of depolarized mitochondria that follows Parkin translocation to mitochondria.

Taken together these results strongly suggest a novel model for the coordination of PINK1 and Parkin in response to mitochondrial damage (FIG. 7). In this model, PINK1 is rapidly turned over on healthy mitochondria with a strong membrane potential by constitutive proteolytic cleavage. On damaged mitochondrial that have lost their membrane potential, however, PINK1 cleavage is blocked with continued synthesis and import leading to its selective accumulation on the damaged mitochondria. PINK1 accumulation, in turn, selectively recruits Parkin to the damaged mitochondria. Once recruited, Parkin can trigger the selective elimination of the uncoupled mitochondria from the mitochondrial network.

This model offers a parsimonious explanation for several puzzling observations that have been made since PINK1 was linked to Parkinson's disease in 2004. Full length mitochondrial PINK1 (˜63 kDa) is cleaved into a short ˜52 kDa form, but this short form is unstable and found primarily in the cytosol, raising the questions: why is PINK1 found in both the cytosol and mitochondrial compartments, and which form is active in the PINK1/Parkin pathway. Without wishing to be bound by theory, the present model strongly suggests that full length mitochondrial PINK1 is the active form of PINK1 in the PINK1/Parkin pathway, and that PINK1's unique processing maintains the full length form at low levels on healthy mitochondria so as not to activate the pathway in the absence of mitochondrial damage. Additionally, without wishing to be bound by theory, this model provides an explanation for the seemingly paradoxical observation that 24 hours treatment with the uncoupler valinomycin (used in an attempt to inhibit the TIM22/23 pathway of mitochondrial inner membrane and matrix import) blocks PINK1 processing but fails to block PINK1 import. Without wishing to be bound by theory, the present model suggests that membrane potential is required not for PINK1 import but for maintaining low PINK1 expression on healthy mitochondria. This mechanism couples the collapse of mitochondrial voltage potential following mitochondrial damage to selective PINK1 accumulation on damaged mitochondria.

At present it is unclear which protease(s) mediate the cleavage of PINK1 in mammalian cells, although the intramembrane protease Rhomboid7 appears to be required for this cleavage in Drosophila, raising the possibility that a rhomboid protease may cleave PINK1 in mammalian cells.

Determining how PINK1 cleavage is modulated by membrane potential will require further study. The protease itself may be sensitive to membrane potential and/or the PINK1 cleavage site may be available to the protease only in the presence of a membrane potential. Alternatively, the regulation of PINK1 cleavage by membrane potential may be indirect. That inhibition of PINK1 cleavage by mitochondrial depolarization upregulates the PINK1/Parkin mitophagy pathway also raises the possibility that inhibitors of PINK1's protease might upregulate the pathway and have some therapeutic benefit.

These results indicate that PINK1 induces Parkin recruitment to a particular subset of mitochondria, following its accumulation, and there are several models for how PINK1 might induce Parkin recruitment. In the simplest, as PINK1 accumulates, Parkin may be recruited to mitochondria through a direct interaction with the accumulated PINK1. In support of this model, PINK1 appears to directly bind Parkin at least in some contexts (Abou-Sleiman P M, Muqit M M, McDonald N Q, Yang Y X, Gandhi S, et al. (2006) Ann Neurol 60: 414-419). Alternatively, PINK1 may need to phosphorylate Parkin, a substrate of Parkin, or an adaptor between PINK1 and Parkin, and thereby increase Parkin's affinity for a substrate or receptor on mitochondria. Consistent with a role for phosophorylation in the activation of Parkin, a kinase-deficient version of PINK1 failed to rescue Parkin recruitment to mitochondria in PINK1 null MEFs (even though PINK1 KD appears to be processed identically to wildtype PINK1). It is possible that Parkin may be phosphorylated by PINK1 elsewhere. If direct phosphorylation is sufficient to induce Parkin recruitment to mitochondria, however, it seems difficult to explain how Parkin can be targeted to a particular subset of mitochondria, as appears to occur in cells with a bioenergetically diverse population of mitochondria.

Mutations in Parkin and PINK1 are inherited primarily in a recessive manner, and loss of their function is thought to cause early-onset Parkinson's disease. As reported herein, patient mutations in PINK1 and Parkin disrupt the PINK1/Parkin mitochondrial turnover pathway at distinct steps, consistent with the potential relevance of this pathway for the development of Parkinson's disease.

Mutations in Parkin's UBL or its deletion caused a moderate deficit in Parkin recruitment to depolarized mitochondria and induction of mitophagy. That deletion of the UBL only partially inhibited the recruitment of Parkin to mitochondria suggests that this domain promotes the recruitment of Parkin to mitochondria, but also suggests that it is not absolutely necessary for recruitment or subsequent mitophagy. The UBL likely promotes recruitment of Parkin through interaction with a protein containing a ubiquitin-binding domain, as mutating I44A, which is critical for the interaction between UBLs and UBDs (Hurley et al., Biochem J 399: 361-372), resulted in a recruitment deficit similar to that of UBL domain deletion. The disease-causing mutations R42P, which causes global unfolding by NMR (Safadi (2007) Biochemistry 46: 14162-14169), and A46P lie on either ends of the betapleated sheet containing I44A, suggesting that these mutations may inhibit Parkin recruitment by disrupting the interaction between Parkin and UBD-containing proteins (FIGS. 21A and B).

Mutations in key cysteine residues in the RBR domain or the deletion of RING2, which is responsible for Parkin's ubiquitin ligase activity, severely disrupt both the recruitment of Parkin to mitochondria and its induction of mitophagy. Interestingly, the R275W mutation in RING1 of Parkin causes only a minor disturbance of Parkin recruitment to depolarized mitochondria but severely disrupts mitophagy, suggesting that recruitment and mitophagy can be experimentally disassociated.

The R275W polymorphism in Parkin and the G411S polymorphism in PINK1 have only been identified as heterozygous mutations in cases of Parkinson's disease (Abou-Sleiman P M, Muqit M M, McDonald N Q, Yang Y X, Gandhi S, et al. (2006) Ann Neurol 60: 414-419). For this reason, the pathogenicity of these mutations has been a matter of controversy. The results reported herein show that the R275W Parkin mutation, which affects a highly conserved arginine residue, caused a significant loss of Parkin function in the mitophagy assay. This is consistent with in vivo data in Drosophila melagaster, demonstrating that Parkin R275W, unlike wildtype Parkin, fails to compensate for loss of endogenous Parkin. By contrast, PINK1 containing the G411S polymorphism, which is conserved in vertebrates but not invertebrates, could compensate for loss of endogenous PINK1, consistent with the view that PINK1 G411S may be a functional polymorphism and not a disease-causing mutation.

The stringent dependence of Parkin recruitment on PINK1 under depolarizing conditions is a little surprising given that, when overexpressed, Parkin can partially compensate for PINK1 loss in Drosophila and in mammalian cells. How Parkin overexpression compensates for PINK1 loss is not known, but there are several possible explanations. First, there may be mechanisms independent of PINK1 and depolarization that can recruit Parkin to dysfunctional mitochondria. Alternatively, Parkin may serve other functions in the cell that are independent of PINK1 and protect against mitochondrial dysfunction indirectly; or Parkin may function to some degree upon overexpression independently of mitochondrial docking, perhaps effecting mitophagy or other mitochondrial changes from the cytosolic compartment.

Stable loss or knockdown of PINK1 in mammalian cellular models and mice leads to a number of mitochondria-related abnormalities. Mitochondria in these cells or tissues exhibit electron transport chain (ETC) dysfunction, diminished membrane potential, increased reactive oxygen species production, mitochondrial fragmentation, and calcium dysregulation, among other abnormalities. While some of these abnormalities may be a reversible consequence of others—for instance, mitochondrial fragmentation may be due to low membrane potential, and ETC dysfunction and decreased membrane potential may be, in part, a functional consequence of calcium dysregulation—other abnormalities may be due to irreversible dysfunction of specific mitochondrial proteins. For instance, Complex I and the putative Na⁺/Ca2⁺ transporter seem to be dysfunctional in cultured cells following PINK1 knockdown, while Complex I and II appear to be dysfunctional in the striatum of mice lacking PINK1.

While the proximate cause of these abnormalities in PINK1 null cells remains obscure, one explanation may be the failure of PINK1/Parkin pathway to eliminate oxidatively damaged mitochondria, which accumulate over time as a natural consequence of metabolism and other cellular stresses. That Parkin null cells and tissues appear to share some of the same mitochondrial defects as PINK1 null cells and tissues supports the view that these abnormalities may be due to loss of a common PINK1/Parkin pathway. It cannot be ruled out that PINK1 may actively prevent mitochondrial damage and dysfunction, in addition to its signaling role in the PINK1/Parkin pathway. PINK1's interaction with HtrA2/OMI, for instance, appears to be independent of Parkin function in Drosophila.

Loss of PINK1 and Parkin affects some cell populations, like substantia nigra neurons, greater than others, even though PINK1 and Parkin appear to be more widely expressed. Why some tissues are more vulnerable to loss of PINK1/Parkin than others is unclear, but it may relate to the degree of damage mitochondria sustain within that tissue (e.g., mitochondria in substantia nigra are subject to greater oxidative stress than those in other neural tissues); the existence of redundant mitophagy pathways (e.g., mammalian tissues may contain pathways orthologous to those recently identified in yeast); the ability of the tissue to mitigate the damage by other means (a tissue composed of mitotic cells may be able to manage mitochondrial damage through cellular turnover rather than mitochondrial turnover); and mitochondrial demand within a particular tissue (neurons have high, local metabolic demands and dopaminergic neurons are subject to especially high calcium fluxes that need to be buffered by mitochondria). Some or all of these factors may contribute to the special reliance of substantia nigra neurons on PINK1 and Parkin.

PINK1 and Parkin are a significant cause of autosomal recessive parkinsonism and have been genetically linked to a pathway that protects against progressive mitochondrial damage and dysfunction. PINK1 levels and consequently Parkin recruitment to mitochondria are dramatically regulated by the bioenergetic state of individual mitochondria, and this unique regulation may allow PINK1 and Parkin to promote the selective and efficient turnover of mitochondria that have become damaged. Loss of PINK1 or Parkin function due to pathogenic mutations can disrupt this mitochondrial turnover pathway which may lead to the accumulation of dysfunctional mitochondria in vulnerable tissues—with a resultant increase in oxidative stress, depression of metabolism, and, eventually, accelerated cell death, all of which has been observed in Drosophila and, to a lesser extent, in mouse models of the disease. Together these findings provide a biochemical explanation for the genetic epistasis between PINK1 and Parkin observed in Drosophila, and support a novel, testable model of how loss of PINK1 and Parkin function may lead to autosomal recessive parkinsonism.

### Example 3

### Parkin Overexpression Selectively Eliminated Mitochondria Containing High Levels of Mutant mtDNA

As reported in the previous examples, the cytosolic E3 ligase, Parkin, which is commonly mutated in autosomal recessive juvenile parkinsonism and has been linked to mitochondria maintenance, can translocate to depolarized mitochondria and activate their elimination by autophagy. In cells containing a mixed population of functional and dysfunctional mitochondria, Parkin selectively localizes to uncoupled mitochondria suggesting that Parkin may function in a mitochondrial quality control process.

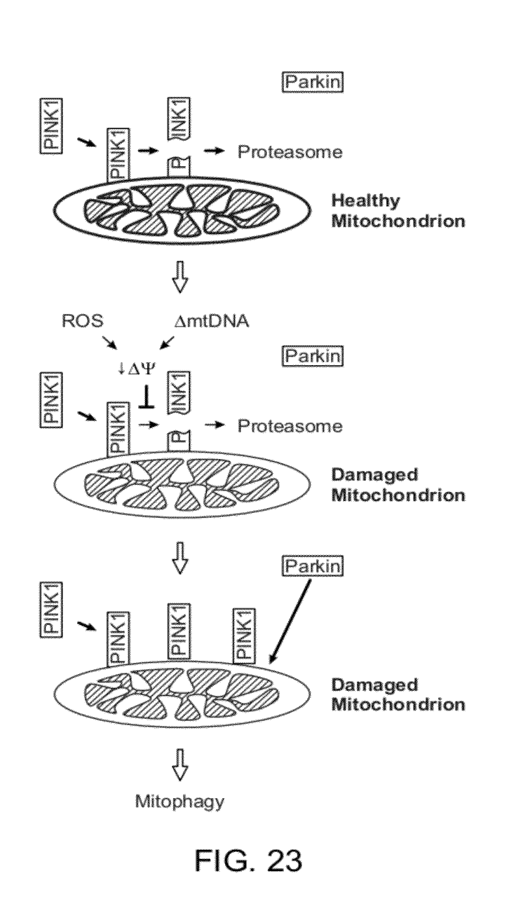

To explore the hypothesis that Parkin can selectively target mitochondria with deleterious mtDNA mutations, Parkin translocation to mitochondria was examined in cells expressing a catalytically inactive form of the mitochondrial DNA helicase, Twinkle. Mutations in Twinkle, which disrupt mtDNA replication and lead to multiple mtDNA deletions, can cause dominant progressive external opthalmoplegia (adPEO), parkinsonism, as well as other symptoms. In addition, overexpression of catalytically inactive Twinkle mutants (such as Twinkle G575D) in cell culture leads to acute loss of mtDNA and mitochondrial dysfunction. Parkin localized to mitochondria appeared twice as frequently in Twinkle G575D expressing cells (13.1±2.3%, mean±s.d.) as in wild-type Twinkle expressing control cells (6.7±1.2%) (FIG. 23A). It is also noteworthy that mitochondria usually appeared reduced in number and clumped upon Parkin translocation in the Twinkle G575D expressing cells consistent with mitochondrial phenotypes observed in response to Parkin upon mitochondrial uncoupling¹⁵. These results suggest that depletion of wild-type mtDNA may induce Parkin recruitment to the resulting dysfunctional mitochondria.

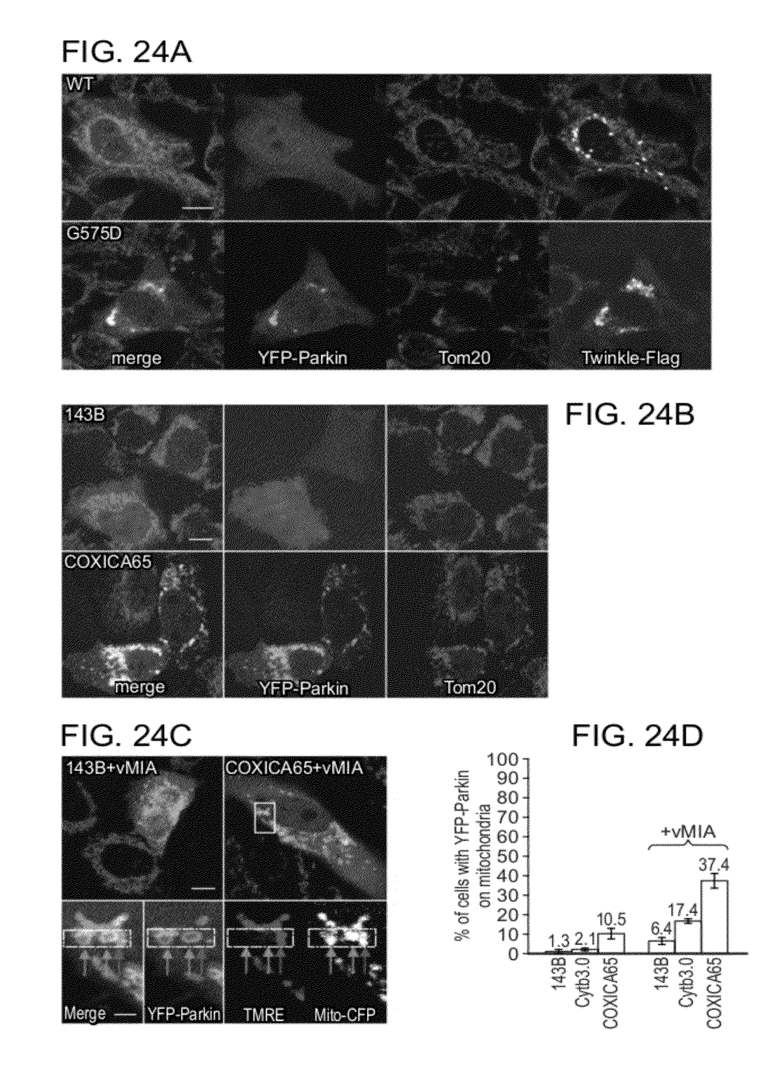

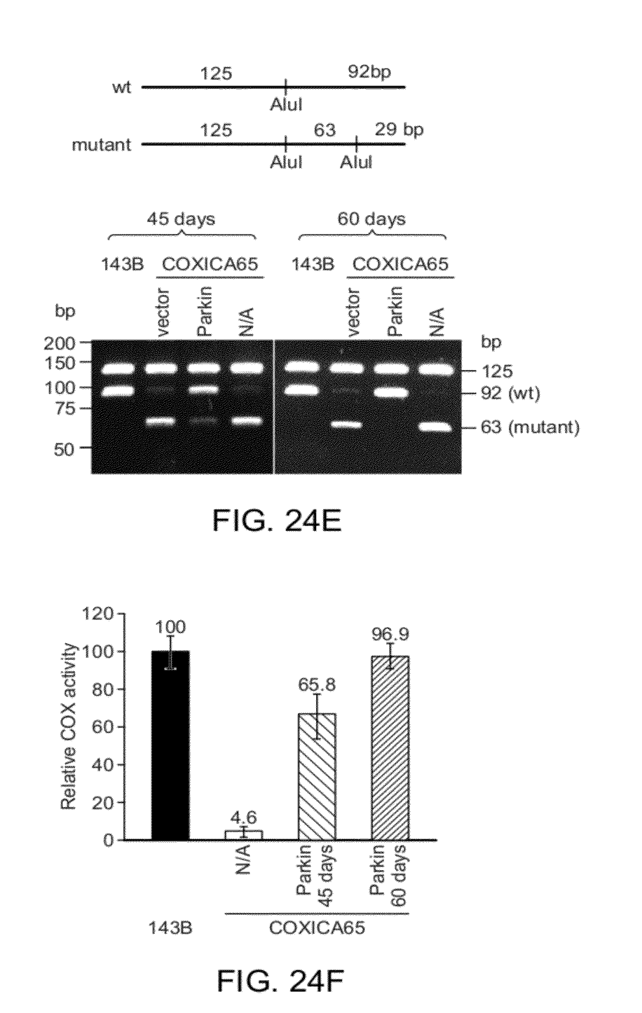

Parkin translocation to mitochondria was examined in cybrid cells possessing a stable mixture of wild-type and mutant mtDNA genomes. In the wild-type parental human osteosarcoma cell line (143B) YFP-Parkin was located on mitochondria in less than 2% of the cells (FIG. 23B, D). In a cybrid cell line containing (need to clarify if this was heteroplasmic, and if so how much) mtDNA mutated in the cytochrome b gene (Cytb3.0)²⁰ only 2.1±0.48% of cells display mitochondrial YFP-Parkin, not significantly different from that of the parental cell line (p=0.32, Student's t-test). However, in a cybrid line containing mtDNA mutated in the cytochrome c oxidase subunit I gene (COXICA65)(Bruno et al. Am J Hum Genet 65, 611-20 (1999)) a significant increase in cells displayed Parkin constitutively localized on mitochondria relative to the parental 143B cells (10.53±2.71% vs. 1.32±1.2%, p=0.006) (FIG. 23B, D). Assessed by flow cytometry, COXICA65 cybrid cells have a lower mean TMRE intensity than Cytb3.0 cybrid cells have (FIG. 24) consistent with the hypothesis that the extent of Parkin recruitment to mitochondria correlates inversely with their mean mitochondrial membrane potential.

Mitochondria frequently fuse and divide potentially allowing wild-type mitochondria to compensate for defects in mutant mitochondria by the transfer of wild-type RNA or protein. To assess if mitochondrial fusion may help maintain mutant mitochondrial membrane potential and prevent Parkin translocation to mutant mitochondria mitochondrial fusion was inhibited by expression of the human cytomegalovirus protein vMIA (viral mitochondrial inhibitor of apoptosis) (McCormick et al., J Virol 77, 631-41, 2003). vMIA expression increased the percentage of cells with Parkin on mitochondria in wild-type, Cytb3.0 cybrid and COXICA65 cybrid lines reaching 37.4±3.87% of cells in the latter case (FIG. 23D). In COXICA65 cells with the interconnectivity of mitochondria reduced by vMIA expression, YFP-Parkin accumulated selectively on mitochondria that displayed less membrane potential detected by TMRE staining (FIG. 23C) consistent with previous results obtained in Mfn1, Mfn2 double knock out cells. This indicates that inhibiting mitochondrial fusion in heteroplasmic cells may physically isolate mitochondria containing high levels of mutant mtDNA from mitochondria containing high levels of wild-type mtDNA, and prevent functional complementation among organelles. Therefore, loss of fusion resulted in increased numbers of depolarized mitochondria, thereby augmenting Parkin translocation to mitochondria in cybrid cells containing mitochondria with high levels of deleterious mtDNA mutations.

To test the hypothesis that Parkin translocation mediates elimination of dysfunctional mitochondria the ratio of wild-type to mutant mtDNA in the COXICA65 cybrid cells was analysed before and several weeks following YFP-Parkin expression by the restriction fragment length polymorphism of PCR product (PCR-RFLP) (FIG. 23E). Parental 143B cells display a single band at 92 bp of digested PCR product amplified from wild-type mtDNA whereas untransfected (N/A) cybrid cells display a minor band at 92 bp and a major band at 63 bp (FIG. 23E). Following transfection and fluorescent activated cell sorting (FACS) to select for YFP transfected cells, COXICA65 cybrid cells expressing the YFP vector (YFP-N1) for 45 days display a minor band derived from wild-type DNA at 92 bp and a major band at 63 bp derived from the mutant genome consistent with the ratio of wt and mutant mtDNA of untransfected cybrid cells (FIG. 23E). In contrast, cybrid cells expressing YFP-Parkin for 45 days display an increase in wild-type DNA and a decrease in mutant DNA (FIG. 23E). After sixty days of culturing YFP-Parkin expressing cells only a very minor band of mutant mtDNA at 63 bp was detected indicative of a strong selection against the mutant DNA (FIG. 23E).

Examining Cytb mutant cybrid cells revealed no such selection of wild-type DNA (data not shown) perhaps owing to a less severe deficit in membrane potential caused by the mutation in Cytb relative to that in COXICA65 (FIG. 24). This failure of Parkin mediated selection for wild-type DNA in Cytb cybrid cells relative to that seen in COXICA65 cybrid cells is consistent with the lack of Parkin recruitment to mitochondria in Cytb cybrid cells relative to that seen in COXICA65 cybrid cells (FIG. 23B, 23D).

Cytochrome c oxidase enzyme activity was analyzed in parental, cybrid and Parkin expressing cybrid cells (FIG. 23F). Whereas COXICA65 cybrid cells had only 4.64±2.80% of the COX activity of parental cells, cybrid cells expressing YFP-Parkin for 60 days had COX activity restored to that of the parental 143B cells (96.90±8.49%). Consistent with increased level of wild-type mtDNA (FIG. 23E), COX activity in cybrid cells expressing YFP-Parkin for 45 days showed a level of COX activity (65.8±16.14%) intermediate between parental and that of the 60 day YFP-Parkin cybrid cultures. These results indicate that Parkin overexpression has the capacity to selectively eliminate dysfunctional mitochondria and allow the repopulation of cells with functional DNA genomes.

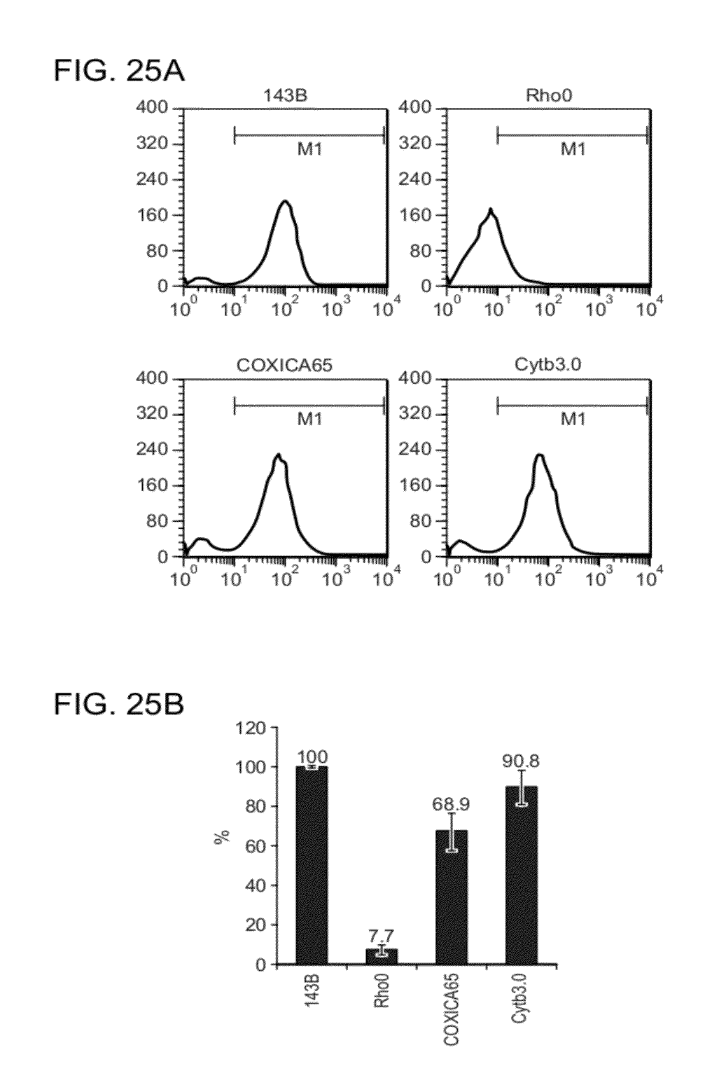

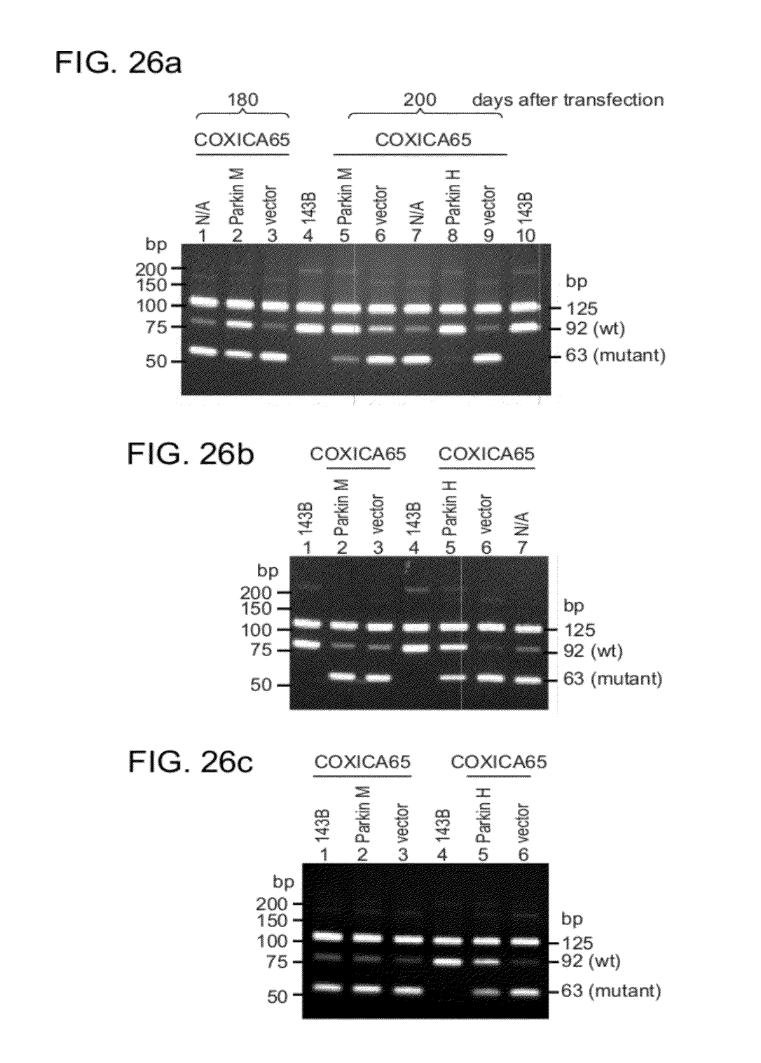

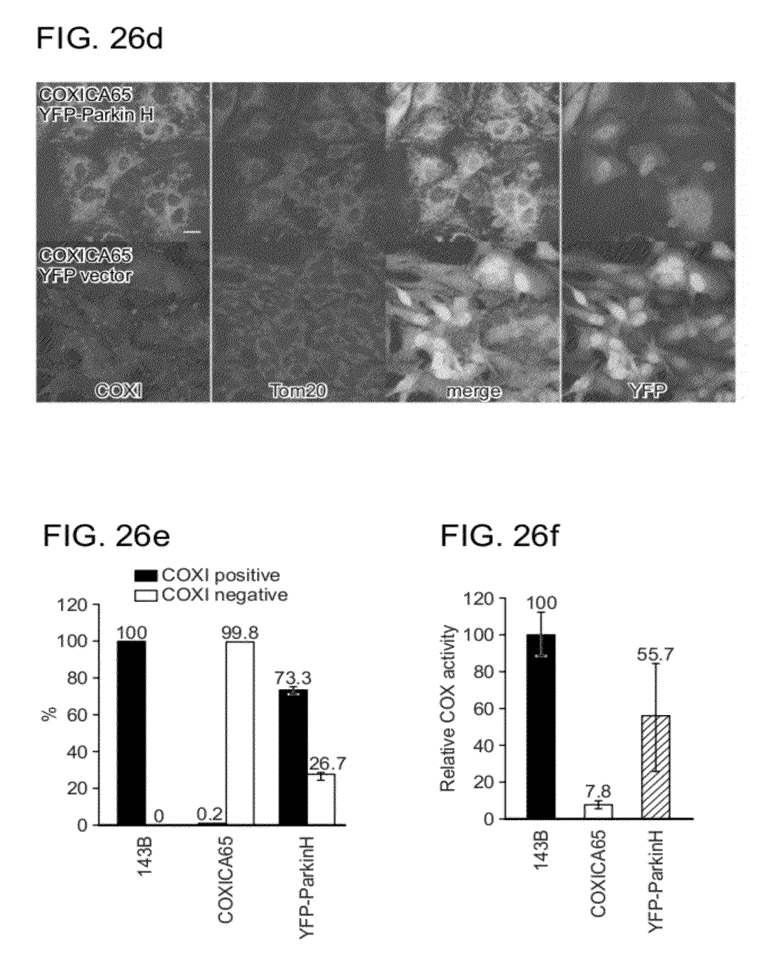

In two additional independent experiments, cybrid cells overexpressing Parkin became enriched for wild-type mtDNA relative to COXI mutant mtDNA after 180 and 200 days of culturing with repeated enrichment for YFP by FACS (FIG. 25A). Quantification of mtDNA by ³²P labeling yielded 15.3% to 21.3% wild-type genomes in both untransfected and YFP vector transfected cells (FIG. 26). Quantification of the Parkin overexpressing cells showed that the percent of wild-type mtDNA increased from ˜20% in the cybrid cells prior to Parkin transfection to 40.3% at 180 days (FIG. 26 lane 1) and to 73.7% after 200 days (FIG. 26 lane 4) in the first experiment and to 90.3% after 200 days (FIG. 26 lane 7) of Parkin overexpression in the second experiment.

Using these two cybrid cell lines enriched for wild-type mtDNA (FIG. 26, lanes 4 and 7), the question of how durably wild-type mtDNA is maintained in the absence of selection for YFP-Parkin expression was explored. After culturing the cybrid cells enriched in wild-type mtDNA by YFP-Parkin expression for a further 40 days and 67 days in the absence of FACS selection for YFP-Parkin, considerable reversion toward mutant mtDNA was observed (FIG. 2B, C). Cells that displayed partial enrichment of wild-type mtDNA (FIG. 25A lane 5, FIG. 26 lane 4) reverted by 40 days to the ratio of wild-type to mutant mtDNA displayed in the untransfected or vector transfected cybrid cell lines (FIG. 25B lane 2) and remained stable at the ratio of untransfected control cybrid cells after 67 days (FIG. 25C, lane 2). However, the cells with greater enrichment in wild-type mtDNA (FIG. 25A lane 8) displayed more durable maintenance of wild-type mtDNA (FIG. 25B, C lanes 5). The ratios of wild-type to mutant mtDNA was quantified in these experiments (FIG. 26). When greater than 90% of mutant mtDNA was eliminated by Parkin (FIG. 26, lane 7), long term enrichment of wild-type mtDNA was maintained following 67 days of culturing with more than 50% wild-type relative to mutant mtDNA (FIG. 26, lane 13). However, if wild-type DNA constituted 73.7% of the total mtDNA (FIG. 26 lane 4), the ratio of mutant to wild-type mtDNA reverted back to the ˜20% wild-type mtDNA percentage of the original cybrid cells (FIG. 26 lane 10). These results indicate that, if insufficient elimination of mutant COXI DNA is achieved, the cells reaccumulate mutant mtDNA. Additionally, they suggest that the COXI mutant genome may have a replicative advantage over wild-type mtDNA.

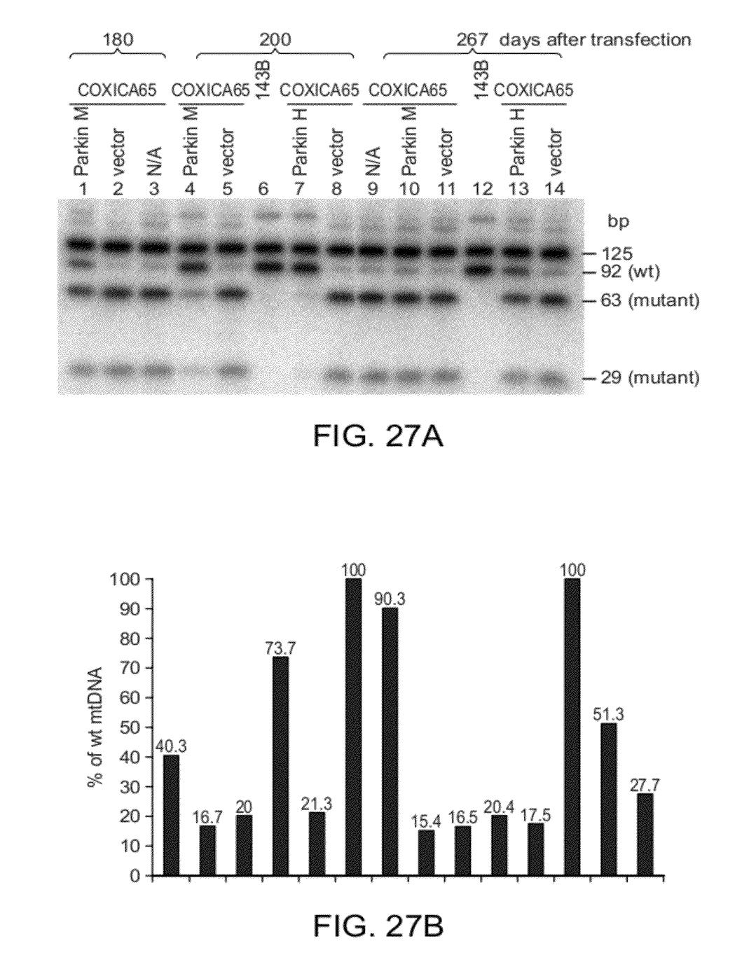

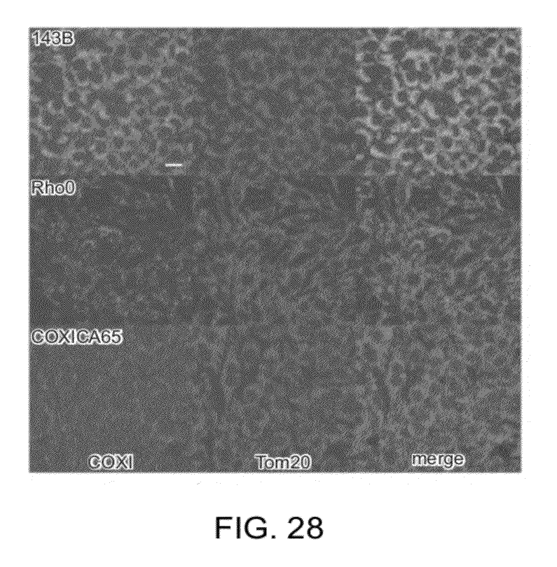

Without wishing to be bound by theory, it is possible that the stable partial (˜50%) selection for wild-type mtDNA (FIG. 26 lane 13) represented a mixture of cells that had completely reverted to the wild-type to mutant mtDNA ratio of the parental cybrid cells and cells that had achieved complete elimination of mutant mtDNA. Cybrid cells that were Parkin-enriched for wild-type mtDNA were immunostained (FIG. 26 lane 13) following 67 days in culture without selection for YFP. Those cells that displayed undetectable YFP-Parkin expression were analyzed for cytochrome c oxidase subunit I (COXI) (FIG. 25D, E and FIG. 27). Approximately 73% of these cybrid cells enriched in wild-type mtDNA were COXI positive and 27% completely negative (FIG. 2E), whereas the original cybrid cells were 99.8% COXI negative (FIGS. 25 and 27). These results are consistent with the hypothesis that the cybrid cells stably enriched to ˜50% wild-type mtDNA by Parkin overexpression comprise a mixed population, consisting of cells that have reverted back to the original cybrid ratio of mainly mutant mtDNA and cells unable to revert owing to complete elimination of mutant mtDNA genomes. c COX activity was assayed in cells stably expressing ˜50% wild-type mtDNA (FIG. 25B, C lanes 5 and FIG. 26 lane 13). They expressed 55.7±29.2% of the COX activity of the parental 143B cell line (FIG. 23F).

Artificially increasing mitochondrial fission with vMIA likely augments Parkin identification of impaired mitochondria presumably by inhibiting compensation of mitochondria with mutant genomes by mitochondria with wild-type genomes. However, without experimentally promoting mitochondrial fission, or artificially depolarizing mitochondrial with uncouplers, over a period of months Parkin can identify most of the mutated mitochondria and activate their elimination. Perhaps this occurs during the normal fission and fusion cycle when mutant mitochondria are segregated from wt mitochondria or this may be augmented by mitochondrial dysfunction fostering selective fission of damaged units²⁵. If the elimination of mutant mtDNA is only partial, in the absence of selection for Parkin overexpression the wild-type mtDNA enriched cells rapidly revert to the original cybrid ration of ˜20% wild-type to ˜80% mutant mtDNA ratio (same issue as above). However, with strong Parkin-mediated enrichment to over 90% wild-type mtDNA, stable selection of COXI positive cells is maintained for months perhaps reflecting complete elimination of mutant mtDNA in many of the cells.

Parkin can translocate selectively to a subset of impaired mitochondria in a cell and that overexpression of Parkin can eliminate all mitochondria by mitophagy when they are chemically uncoupled. On the basis of these findings, it was hypothesized that Parkin may mediate an organelle quality control pathway. The experiments presented here, designed to test this hypothesis, show that Parkin overexpression has the capacity to selectively eliminate mitochondria containing high levels of mutant mtDNA. These results support the proposal that Parkin may normally select for healthy mitochondria with wild-type mitochondrial DNA by mediating the selective elimination of dysfunctional mitochondria. Loss of Parkin function in the substantia nigra may cause early onset Parkinsonism by allowing the excessive accumulation of deleterious mutant mtDNA at earlier ages than normally occurs during aging. Importantly, these findings also indicate that endogenous Parkin levels may be limiting for the negative selection of dysfunctional mitochondria in at least some cell types, and that upregulation of Parkin expression may be therapeutically beneficial for hereditary and somatically acquired mitochondrial diseases.

### Example 4

### PARL is Required for Proteolytic Cleavage of PINK1

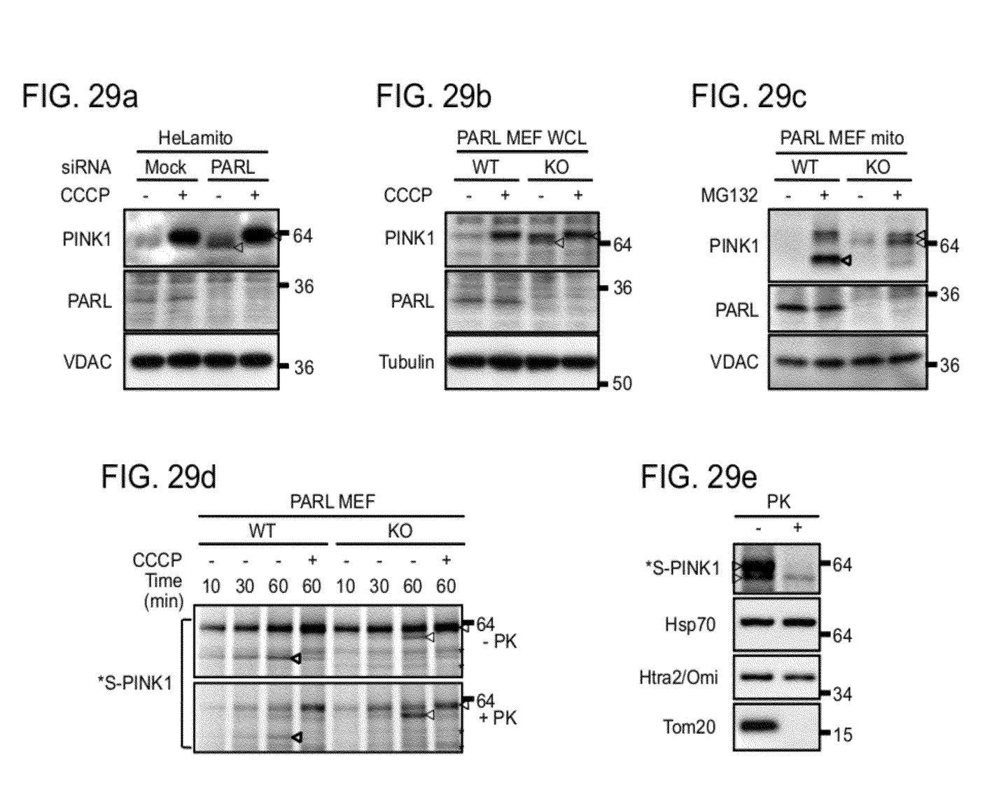

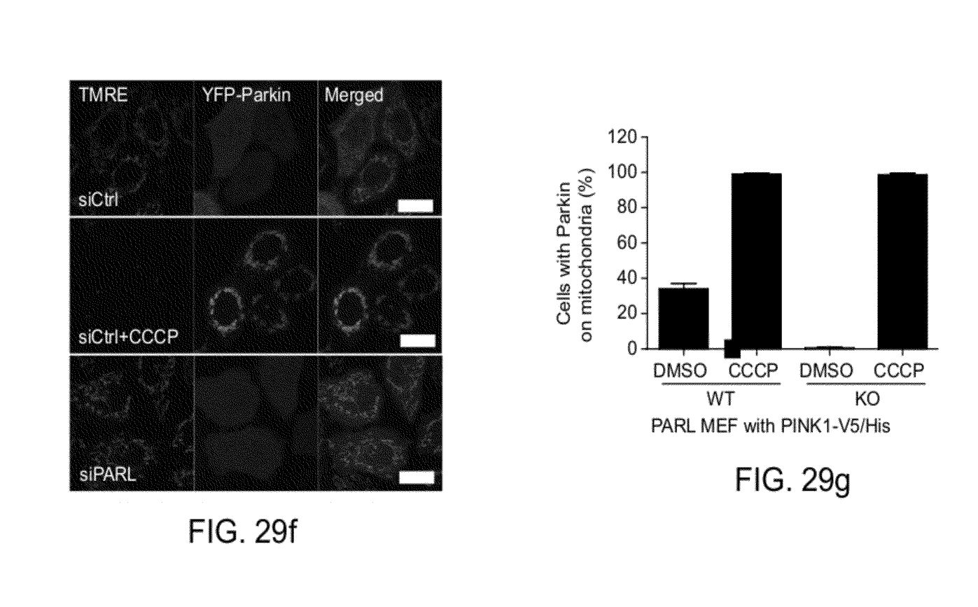

To identify the protease mediating PINK1 turnover on mitochondria, known mitochondrial proteases were knocked down by siRNA and the PINK1 expression pattern was examined. In contrast to siRNAs against Afg3L2, ClpP, Oma1, HtrA2/Omi, Paraplegin and Yme1, siRNA for PARL led to increased expression of endogenous PINK1 in the absence of the depolarizing agent, carbonyl cyanide-m-chlorophenyl hydrazone (CCCP) (FIG. 29A). In addition, the molecular weight of the PINK1 band in the absence of CCCP was slightly lower than that of endogenous PINK1 stabilized by CCCP predicted to represent full-length (FL) PINK1 (63 kDa) based on molecular weight. The ˜60 kDa band would be consistent with the molecular weight of PINK1 lacking a mitochondrial targeting sequence (ΔMTS) following MPP cleavage (FIG. 29A). When mitochondria were uncoupled with CCCP, both scrambled siRNA and PARL siRNA transfected cells showed the same level of FL PINK1 accumulation, indicating that knockdown of PARL did not affect PINK1 expression in uncoupled mitochondria. This contrasts with previous work using shRNAi targeting a different site in PARL mRNA that was interpreted to exclude PARL as a protease for PINK1 cleavage, likely owing to insufficient knock down of endogenous PARL in HeLa cells and to insufficient expression of ectopic PINK1 in PARL KO MEFs (Narendra et al., 2010, PLoS Biol. 8:e1000298.). To confirm the effect of PARL knockdown on endogenous PINK1 cleavage, mouse embryonic fibroblasts (MEF) derived from wild type (WT) or PARL knockout (KO) mice were analyzed. Given that endogenous mouse PINK1 could not be detected in MEF cells, cells were transfected with a human PINK1-V5/His construct. Consistent with the results observed in HeLa cells for endogenous PINK1, PARL KO MEFs displayed a 60 kDa form of PINK1, likely to be ΔMTS-PINK1, in the absence of CCCP (FIG. 29B).

Treatment of cells with a proteosome inhibitor stabilizes a 52 kDa PINK1 fragment (Lin and Kang, 2008; J Neurochem. 106:464-74; Zhou et al. 2008, Proc Natl Acad Sci USA. 105:12022-7), suggesting that proteosome-independent proteolysis yields a 52 kDa form of PINK1 that is subsequently degraded by the proteosome (Narendra et al., 2010, PLoS Biol. 8:e1000298). When PARL KO MEFs transiently overexpressing PINK1-V5/His were treated with MG132, the 52 kDa fragment of PINK1 (red arrow) was absent and a 60 kDa, predicted ΔMTS-PINK1, was stabilized (FIG. 29C), suggesting that PARL-mediated proteolysis normally generates the 52 kDa fragment. Rhomboid proteases such as PARL cleave proteins in and around membrane spanning domains (Sik et al., 2004; J Biol Chem. 279:15323-9; Urban et al., 2001, Cell. 107:173-82; Strisovsky et al., 2009, Mol Cell. 36:1048-1059.). PARL mediated cleavage of PINK1 in the predicted membrane spanning domain between residues 94 and 110 would yield a protein fragment of 52 kDa consistent with the molecular weight of the fragment stabilized by MG132 and absent in the PARL KO MEFs.

To further analyze the effect of PARL on PINK1 cleavage, in vitro mitochondrial import assays were performed. Mitochondria freshly isolated from WT and PARL KO MEFs were incubated with ³⁵S-labeled PINK1 in the presence or absence of CCCP. Following import, each sample was split in two and treated with or without Proteinase K (PK; 5 μg/ml) to degrade non-imported protein (FIG. 29D). The 52 kDa species of PINK1 was found to accumulate in WT MEF mitochondria (red arrows) but was absent in PARL KO samples, corroborating that PARL-mediated proteolysis generates the 52 kDa form. As in PARL KO cells (FIG. 29C), mitochondria isolated from PARL KO MEFs did not generate the 52 kDa fragment but yielded a new 60 kDa species of PINK1 predicted to be ΔMTS-PINK1. Generation of both the 52 kDa form of PINK1 in WT mitochondria and the 60 kDa form of PINK1 in PARL KO mitochondria was prevented by CCCP, indicating that they required inner mitochondrial membrane import (FIG. 29D). When the mitochondria containing imported ³⁵S-labeled PINK1 were incubated with PK, the 60 kDa ΔMTS-PINK1 in PARL KO mitochondria and the 52 kDa PINK1 in WT mitochondria were more stable than FL PINK1 (FIG. 29D, bottom panel). These results indicate that in contrast to the FL, CCCP-stabilized form of PINK1 that localizes to the outer mitochondrial membrane facing the cytosol (Narendra et al., 2010, PLoS Biol. 8:e1000298), the 60 kDa ΔMTS-PINK1 that appears in the absence of PARL and the 52 kDa PINK1 are protease-protected within polarized mitochondria (FIG. 29D).

To confirm that ΔMTS-PINK1 reflects a form imported into the mitochondria to allow MPP cleavage, PK sensitivity following import of radiolabeled PINK1 was compared to control proteins in the outer mitochondrial membrane and intermembrane space (FIG. 29E). FL radiolabeled PINK and Tom20 were rapidly degraded by PK treatment (100 μg/ml) while ΔMTS-PINK1, intermembrane space protein Htra2/Omi and matrix protein Hsp70 were more stable.

Experimentally stabilized PINK1 on the mitochondrial outer membrane recruits Parkin even in the absence of mitochondrial uncoupling (Narendra et al., 2010, PLoS Biol. 8:e1000298). Therefore, if ΔMTS-PINK1 is sequestered within the mitochondria, as indicated above, it would not be positioned to recruit Parkin. Indeed, when HeLa cells stably overexpressing YFP-Parkin were transfected with scrambled control siRNA or siRNA for PARL as in FIG. 29A, no mitochondrial translocation of Parkin was observed (FIG. 29F). Supporting this conclusion, the 60 kDa form of ΔMTS-PINK1 stabilized in coupled mitochondria in the absence of CCCP in PARL KO MEFs fails to recruit mCherry-Parkin to mitochondria (FIG. 29G).

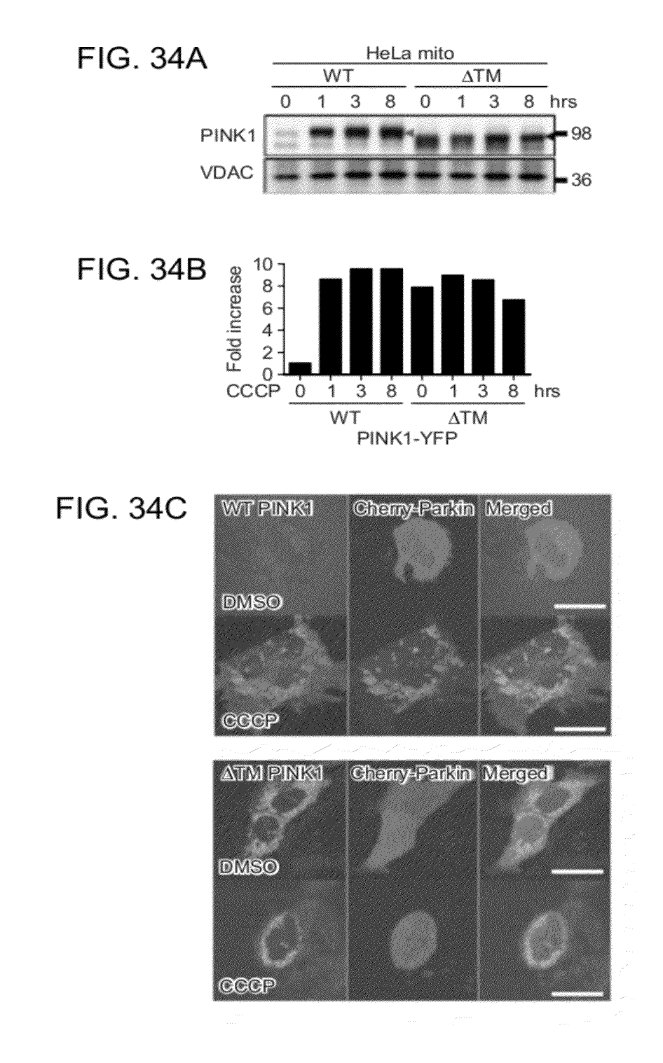

A mutant form of PINK1 lacking the transmembrane region in PINK1 between amino acids 91-117 does not accumulate in cells upon CCCP treatment (FIGS. 34A and 34B) and does not function to recruit Parkin after CCCP treatment in marked contrast to WT PINK1 (FIG. 34C). Thus, the transmembrane domain appears to be essential for proper positioning of WT PINK1 in the outer mitochondrial membrane to recruit Parkin.

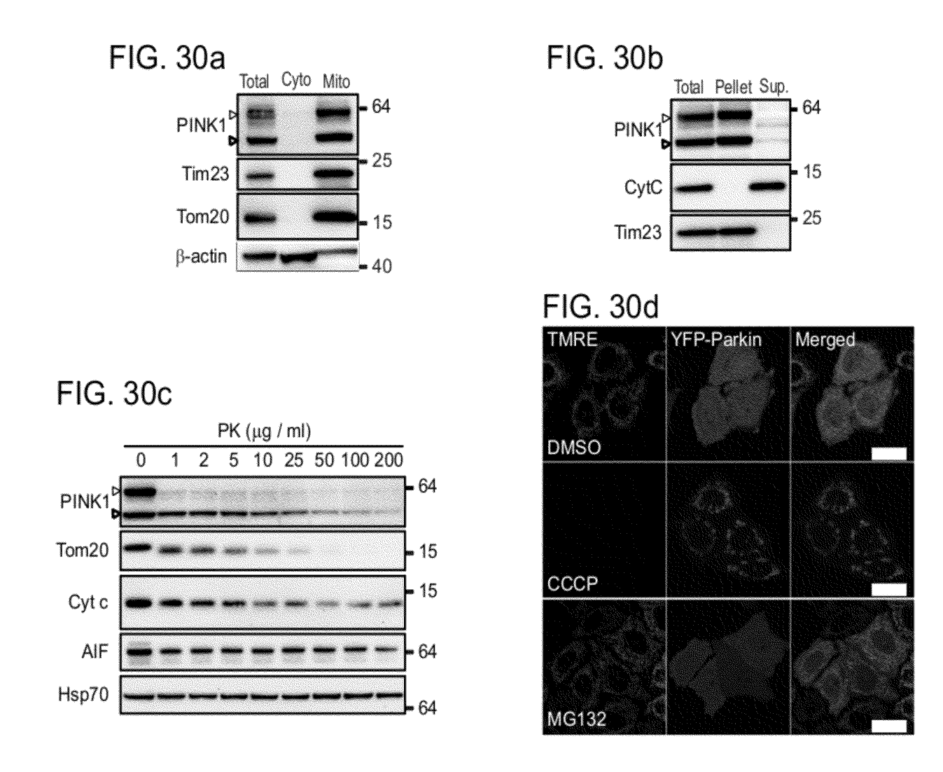

Although overexpressed PINK1 accumulates as a 52 kDa form in the cytosol following proteosome inhibition (Lin and Kang, 2008, J Neurochem. 106:464-74; Muqit et al., 2006, J Neurochem. 98:156-69; Takatori et al., 2008, Neurosci Lett. 430:13-7; Tang et al., 2006, Hum Mol Genet. 15:1816-25; Weihofen et al., 2008, Hum Mol Genet. 17:602-16), the location and topology of the endogenous 52 kDa PINK1 produced by PARL-mediated proteolysis has not been conclusively elucidated. To address this, cells were treated first with MG132 in order to accumulate the 52 kDa PINK1 followed by treatment with CCCP to accumulate FL 63 kDa PINK1 to compare the localization of the two proteins in the same samples. Endogenous FL PINK1 and 52 kDa PINK1 were detected in the mitochondrial fraction but not in the cytosolic fraction (FIG. 30A). Mitochondrial fractions subjected to alkaline (Na2CO3) extraction yielded FL and 52 kDa PINK1 in the pellet fraction, suggesting that both forms of PINK1 are integrated within mitochondrial membranes (FIG. 30B). In order to determine whether FL and 52 kDa PINK1 exist in the same sub-mitochondrial compartment a PK protection assay was conducted. While FL PINK1 was rapidly degraded by low concentrations of PK (1 μg/ml), the 52 kDa PINK1 was very stable and could be detected even after incubation with 100 μg/ml PK (FIG. 30C), indicating that endogenous FL PINK1 and 52 kDa PINK1 are in different compartments corroborating the in vitro import results in FIG. 1d. The activity of endogenous 52 kDa PINK1 to recruit Parkin was addressed. When HeLa cells stably expressing YFP-Parkin were treated with MG132 for 8 hrs to accumulate the 52 kDa PINK1 (FIG. 29C, see Narendra et al., 2010, PLoS Biol. 8:e1000298), no mitochondrial translocation of Parkin was detected (FIG. 30D) consistent with import and PARL-mediated cleavage of PINK1 in the inner mitochondrial membrane in the absence of uncoupling. Although the 52 kDa form of PINK1 was not functional for Parkin translocation due to its different submitochondrial localization compared to CCCP-stabilized FL PINK1, this fragment may have other functions within the mitochondria.

### Example 5

### Mutagenesis Screen of Amino Acid Residues Affecting PARL-Mediated Cleavage of PINK1

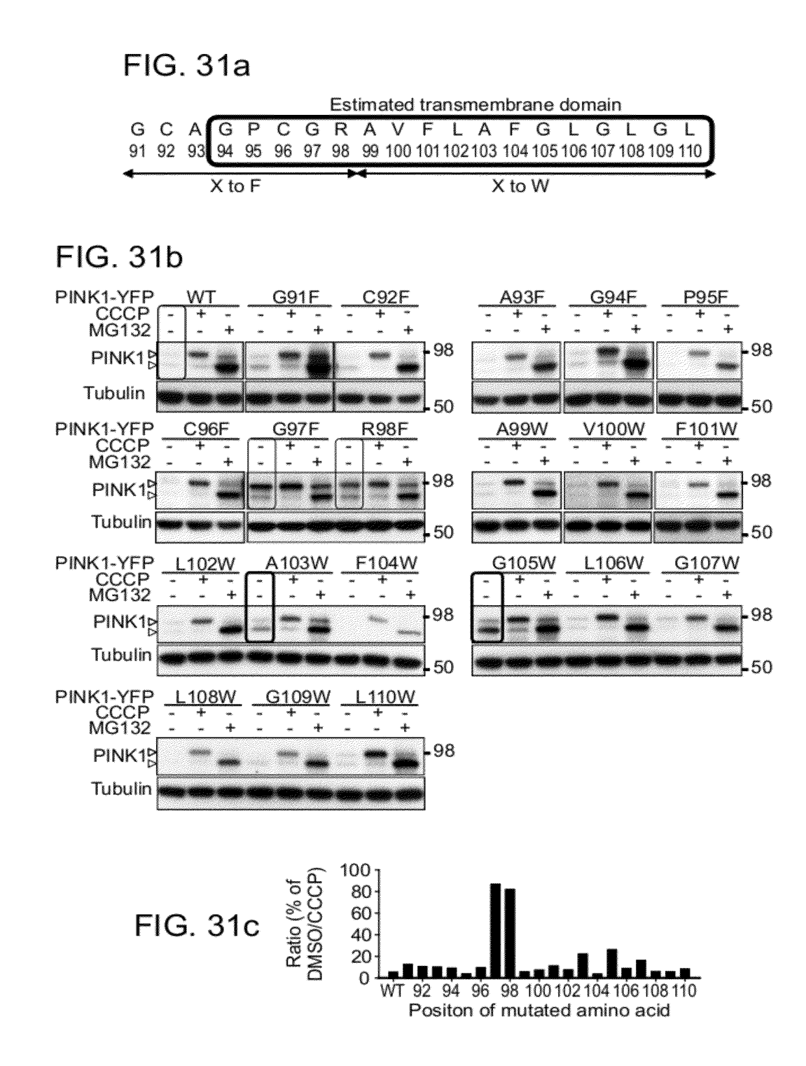

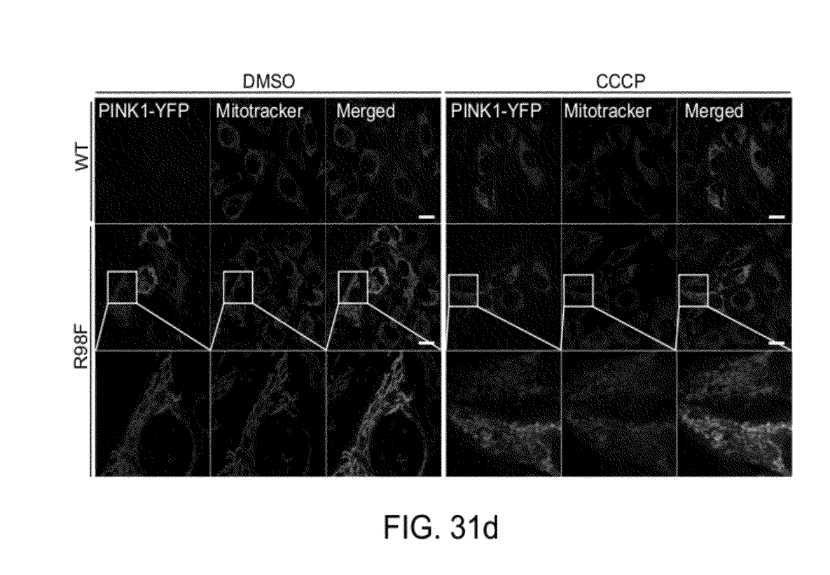

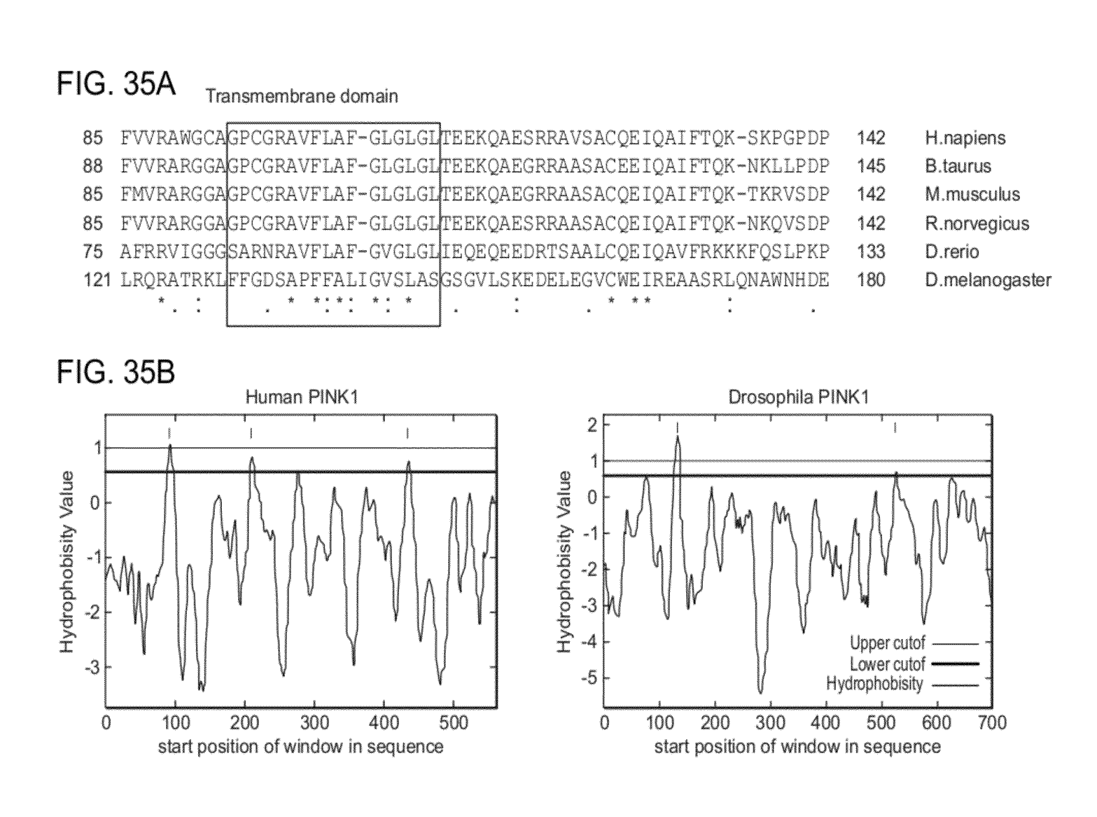

Rhomboid proteases prefer to cleave specific sequences near to transmembrane domains and within membrane spanning helices that are partially destabilized by helix-breaking amino acids such as glycine and proline (Urban and Freeman, 2003, Mol Cell. 11:1425-34; Strisovsky et al., 2009, Mol Cell. 36:1048-1059). Interestingly, the predicted membrane spanning region of PINK1, between Ala⁹³ and Ile¹¹¹, contains more than one third glycine and proline residues (FIG. 31A) consistent with the high susceptibility to PARL cleavage identified. Moreover, the PINK1 transmembrane domain is highly conserved from zebrafish to human (FIG. 35). Drosophila PINK1 has a predicted transmembrane domain that is less homologous to man and contains fewer helix breaking residues suggesting that it may have a different sensitivity to the fly PARL orthologue, Rhomboid 7 (FIG. 35). In order to explore PINK1 cleavage mediated by PARL, each amino acid in this domain was mutated to residues with a bulky side chain that was predicted to interfere with substrate recognition by PARL. N-terminal amino acids 91-98 of WT PINK1-YFP construct were mutated to phenylalanine, and amino acids 99-110 mutated to tryptophan (FIG. 31A).

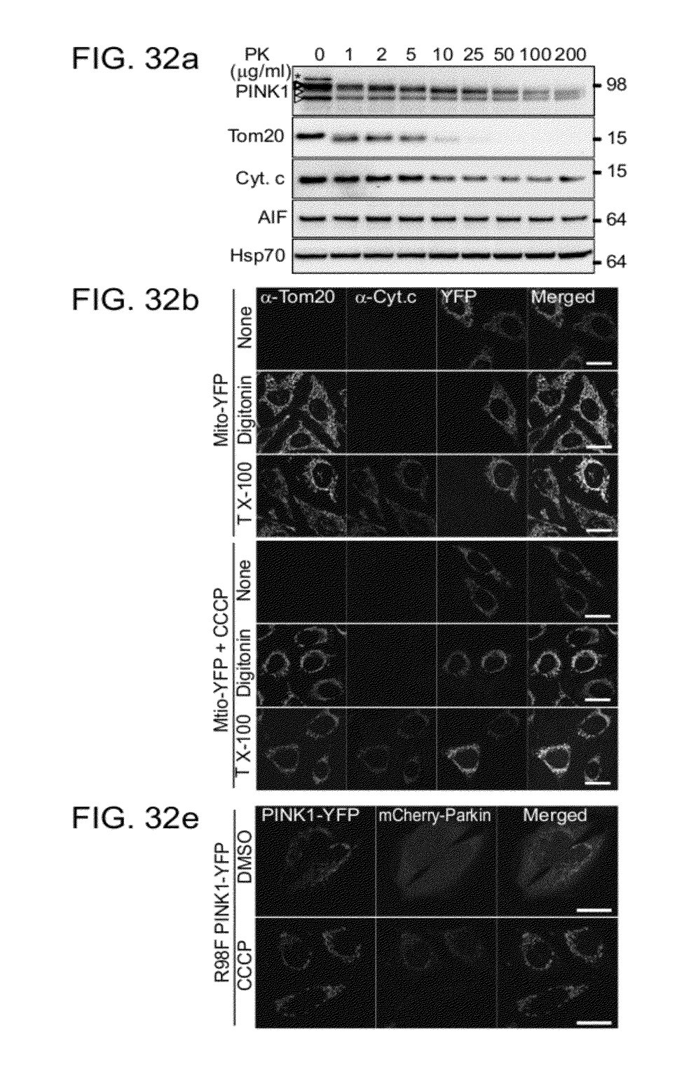

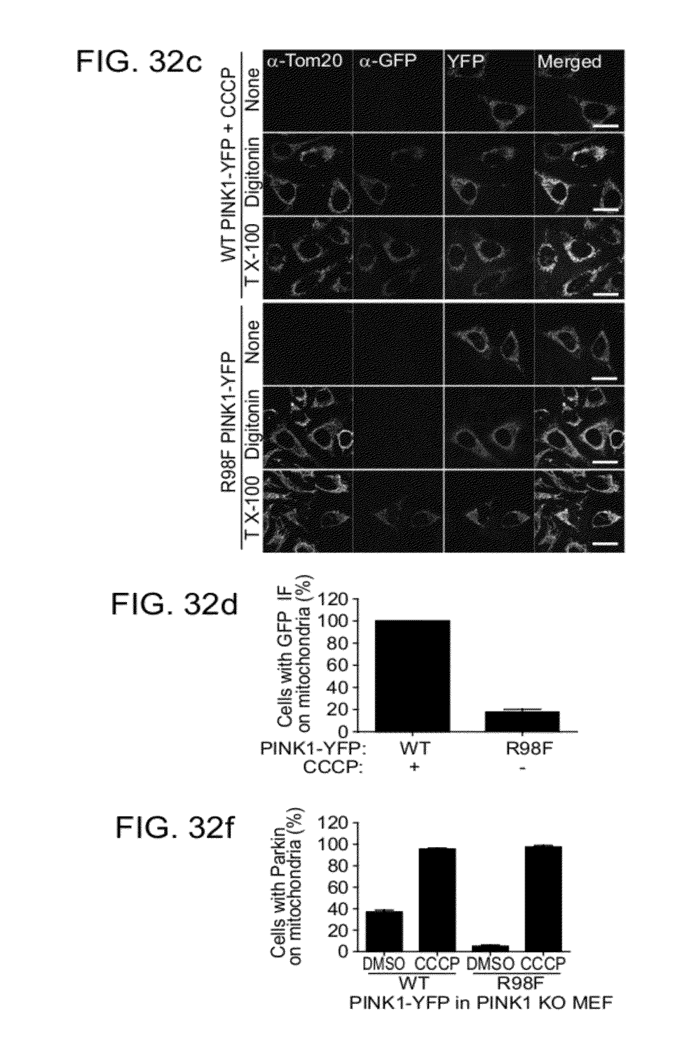

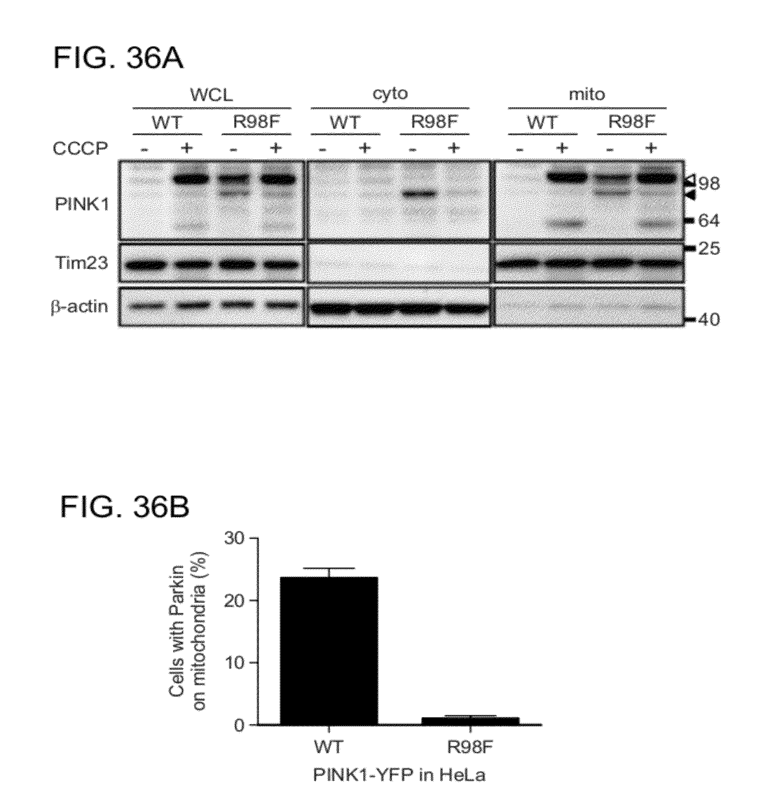

HeLa cells transfected with mutant PINK1-YFP constructs were either untreated, or treated with CCCP or MG132 and analyzed by immunoblotting (FIG. 31B). In the absence of CCCP or MG132, PINK1-YFP mutants G97F and R98F displayed dramatically increased levels of FL and/or ΔMTS-PINK1 (arrow; compare control yellow rectangle with red rectangles) (FIGS. 31B and 31C) in contrast to WT and all the other mutant forms of PINK1. However, in the presence of MG132, G97F and R98F PINK1 mutants also displayed partial cleavage to the 52 kDa form (red arrow), indicating that PARL-mediated PINK1 cleavage was not completely prevented by these mutations.

Whereas WT PINK1-YFP expression is below the level of detection, the PINK1-YFP R98F mutant is found localized to mitochondria in the absence of CCCP based on subcellular fractionation (FIG. 36A) and confocal imaging (FIG. 31D). In order to determine the sub-mitochondrial location of PINK1 R98F, a mitochondrial PK protection assay was performed. Mitochondria isolated from HeLa cells transfected with PINK1-YFP R98F were treated with increasing amounts of PK and the degradation pattern of PINK1 R98F was compared to the degradation patterns of mitochondrial proteins representing each compartment of mitochondria (FIG. 32A). PINK1-YFP R98F (green arrow) exhibited increased protease protection relative to WT PINK1 (see FIG. 30A), suggesting that the mutant is imported but incompletely processed by PARL-mediated proteolytic activity. To further investigate the sub-mitochondrial location of PINK1-YFP R98F, immunostaining was performed on fixed cells that were either untreated or permeabilized with 0.005% digitonin or 0.25% Triton X-100. Control experiments showed that this assay could distinguish between mitochondrial proteins localized inside (cytochrome c; Cyt. c) or outside (Tom20) the mitochondrial outer membrane (FIG. 32B). HeLa cells transfected with WT PINK1-YFP followed by treatment with CCCP for 3 hrs showed positive immunostaining in all cells expressing PINK1-YFP after permeabilization with 0.005% digitonin. However, anti-GFP immunoreactivity was absent in most HeLa cells transfected with R98F PINK1-YFP using the same permeabilization conditions, indicating that the C-terminus of PINK1-YFP R98F is protected by the mitochondrial outer membrane in the absence of CCCP treatment (FIGS. 32C and 32D).

Consistent with localization within mitochondria, expression of R98F PINK1-YFP did not induce mitochondrial translocation of mCherry-Parkin in HeLa cells (FIGS. 32E and 36B) or in PINK1 KO cells (FIG. 32F). CCCP treatment of PINK1 KO cells expressing R98F PINK1-YFP induced Parkin translocation, indicating that this mutant is functional for inducing Parkin translocation when located on the outer mitochondrial membrane.

Interestingly, in the screen for PINK1 mutants displaying incomplete PARL-mediated cleavage, the screen also identified A103W and G105W mutations that instead stabilize the 52 kDa fragment of PINK1 (FIG. 31B) in the absence of MG132 treatment. Using differential permeabilization conditions described in FIG. 32B, both A103W and G105W PINK1 mutants were protected by the mitochondrial outer membrane and did not recruit Parkin to mitochondria in HeLa cells or in PINK1 KO MEFs (data not shown).

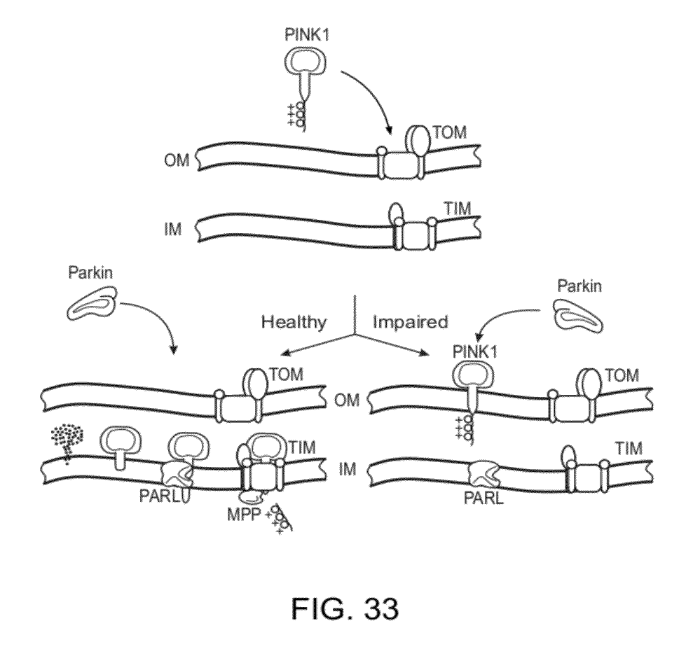

In coupled mitochondria PINK1 appears to be guided to mitochondria by the N-terminal targeting sequence after translation and imported into the inner mitochondrial membrane via the general mitochondrial import machinery, TOM and TIM23 complexes (FIG. 33). Here, it would encounter MPP which cleaves the MTS for most MTS-containing mitochondrial proteins to generate a 60 kDa ΔMTS-PINK1. Then, PINK1 appears to be cleaved to a 52 kDa form within the inner mitochondrial membrane by PARL-mediated proteolytic activity. The 52 kDa PINK1 is then degraded by an MG132-sensitive protease. Thus, a prominent function of PARL in the PINK1-Parkin pathway appears to be facilitating the rapid degradation of PINK1 by mediating the cleavage of PINK1 in the mitochondrial inner membrane. The PARL KO mouse displays defects in postnatal growth and lifespan (Cipolat et al., 2006, Cell. 126:163-175), that may in part be due to accumulation of ΔMTS-PINK1 in the mitochondrial inner membrane space.

These results indicate that a protein with an MTS can be differentially targeted to inner and outer mitochondrial membranes depending on the presence of mitochondrial membrane potential and may explain the discrepancies regarding PINK1 location in the literature. In yeast, a bipartite targeting sequence of NADH-cytochrome b5 reductase (Mcr1p) drives it to the mitochondrial inner membrane where it is cleaved and released into the intermembrane space. Interestingly, in the absence of membrane potential Mcr1p is found predominantly on the outer membrane reflecting the findings with PINK1 (Haucke et al., 1997, Mol Cell Biol. 17:4024-32). It seems plausible that additional proteins will be found that are regulated by differential mitochondrial membrane targeting. Interestingly, expression of full length Oma1 was recently indicated to increase on mitochondrial membrane potential collapse allowing it to accumulate and cleave the mitochondrial fusion protein, Opa1 (Head at el., 2009, J Cell Biol. 187:959-966). Opa1 degradation by Oma1 may prevent fusion of damaged mitochondria with healthy mitochondria and be coupled to Pink1-mediated recruitment of Parkin to facilitate mitophagy.

### Example 6

### PINK1 High Throughput Screening Assay

The PINK1 high throughput assay screen exogenous agents including, but not limited to, small molecules, cDNAs, siRNAs, and shRNAs. Methods for contacting the exogenous agent with a cell are well known in the art. The assay reports the PINK1 protein level, localization, and overall cellular health in the presence of the exogenous agent. By utilizing a combination of immunofluorescence and cell staining, the PINK1 assay provides high content data from cell cytometry and microscopy-based data collection. The PINK1 assay is run in 96, 384, or 1536-well microplates in addition to standard microscopy coverslips and chambers.

PINK1 localization and protein levels in each cell are detected by immunofluorescence from antibodies that recognize epiotpes on the PINK1 protein or epitopes that are expressed as fusions with the PINK1 protein. Mitochondrial morphology is illuminated in each cell by antibodies that specifically recognize abundant mitochondrial proteins (eg., Tomm20, Cytrochrome c, Porin, VDAC, and α-Ketoglutarate dehydrogenase). Immunofluorescent analysis of both mitochondrial morphology and PINK1 can utilize either fluorophore labeled primary antibodies or fluorophore labeled secondary antibodies that recognize the immunoglobulins of the primary antibodies. Additionally, cell nuclei are labeled with stains such as DAPI, DRAQ5, or Hoechst 33342 to achieve cell counts and nuclear segmentation for image analysis.

Immunofluorescent detection can be directed at endogenous PINK1 or stably expressed PINK1 fused to an epitope tag (eg., myc, HA, and FLAG).

One of skill in the art can readily determine the appropriate cell type for use in the PINK1 assay. For example, the PINK1 assay accommodates both immortalized cell lines (eg. HeLa, HCT116, SH-SY5Y, and BE(2)-M17), stem cells, induced pluripotent stem cells, patient-derived fibroblasts, or cultured primary cells.

Cells are seeded into the designated assay vessel (microplates, culture dishes, microscopy chambers, etc.) containing tissue culture medium and allowed to adhere to the optical surface. Test agents are added to the culture medium in conjunction or following the cell seeding. Such agents are queried for the induction of changes in PINK1 expression and/or localization. After an incubation period with the agent, cells are fixed with an aldehyde or alcohol-based solution. During or after the washes to remove the chemical fixative, the cell and mitochondrial membranes are permeablized with a detergent-based solution, and then a blocking buffer is applied to block and prevent the nonspecific binding of primary and secondary antibodies. Suitable blocking buffers are well known in the art, including, but not limited to, bovine serum albumin, reconstituted milk (fat-free), cold fish skin gelatin, or other reagent combinations that ameliorate nonspecific antibody binding. After incubation with the blocking solution, it is removed and primary antibodies are added to detect PINK1 and mitochondrial markers. Following incubation, cells are optionally washed a secondary antibody solution is added (if needed). After the optional secondary antibody incubation, cells are washed and stained (nuclei). Data is generated by quantifying cellular and sub-cellular imaging of PINK1, mitochondrial markers, and cell stains. Image cytometry or microscopy is used to generate the raw data needed to quantify PINK1 levels, localization, cytotoxicity, and mitotoxicity.

The experiments reported above in Example 1 were carried out with the following methods and materials.

### Cell Culture

HeLa cells stably expressing YFP-Parkin using the Flp-In system (Invitrogen) were creating according to the manufacturer's instructions and maintained in 300 μg/ml hygromycin (Sigma-Aldrich). Rat cortical neurons were isolated on embryonic day 18 and grown in neurobasal media supplemented with B-27, L-glutamine, and penicillin/streptomycin. All cell culture materials were obtained from Invitrogen and all chemicals were obtained from Sigma-Aldrich. Chemicals were prepared from DMSO stock solutions, except paraquat, N-acetyl-cysteine, and 3-methyladenine, which were added fresh to media. Mfn1−/−, Mfn2−/− and Mfn1−/−, Mfn2−/− double knockout MEFs were generously donated by D. C. Chan (California Institute of Technology, Pasadena, Calif.), ATG5−/− MEFs were donated by N. Mizushima (Tokyo Medical and Dental University, Tokyo, Japan), Flp-In HeLa cells were donated by V. V. Lobanenkov (National Institutes of Health, Rockville, Md.), and HeLa cells stably expressing GFP-LC3 were donated by A. Tolkovsky (Cambridge University, Cambridge, UK).

### Transfection/Immunocytochemistry

Cultured cells seeded in borosilicate chamber slides (Thermo Fisher Scientific) were transfected or cotransfected with YFP-Parkin, ECFP-Parkin, mCherry-Parkin, DsRed-Mito (Clontech Laboratories, Inc.), pcDNA3.1 (Invitrogen), vMIA, and/or Drp1K38A constructs using Fugene 6 (Roche). Parkin-myc was a gift from M. Cookson (National Institutes of Health, Bethesda, Md.). Cells were fixed 12-24 hours after transfection with 4% paraformaldehyde in PBS. Cells were stained with following primary antibodies: mouse monoclonal cytochrome c (BD), rabbit polyclonal Tom20 (Santa Cruz Biotechnology, Inc.), mouse monoclonal Parkin PRK8 (Santa Cruz Biotechnology, Inc.), rabbit polyclonal PMP70 (Invitrogen), and/or mouse monoclonal TRAP1 (Abcam); and with the following secondary antibodies: mouse and/or rabbit Alexa 488, 594, and 633 (Invitrogen). For assessment of mitochondrial membrane potential, cells were pulsed with 50 nM Mito-Tracker red (Invitrogen) for 15 min, washed, and incubated for an additional 10 minutes before fixation or imaging. For assessment of cell metabolic potential, cell nuclei were stained with Hoechst 33342 (Invitrogen).

### Confocal Microscopy

Fixed cells and live cells in the FLIP assay were imaged using an inverted microscope (LSM510 Meta; Carl Zeiss, Inc.) with a 63×/1.4 oil DIC Plan Apo objective at 25° C. and 37° C., respectively. For the FLIP assay, a bleach region of interest (ROI) occupying approximately one eighth of the cell was positioned over a relatively mitochondria-free portion of the cytosol. Cells were alternately bleached (488 nm using a 30-mW argon laser at 75% power and 100% transmission for 150 iterations) and imaged (488 nm at 75% power and 2% transmission) for 10 minutes (_{—}60 cycles over the length of the experiment). Two channel prebleach and postbleach images were obtained with 488 and 594 lasers to assess the position of mitochondria before and after bleaching. Circular ROIs with diameters of ˜1 and 5 μm, respectively, were placed over the mitochondria and cytosol of the target cell, and an ROI of 10 μm was placed over the control cell. Imaging of YFP-Parkin translocation in live HeLa cells was performed on a live cell imager system (UltraView LCI; PerkinElmer) at 35° C. with a 100×/1.45 α-Plan-Fluor objective.

### Image Analysis

Image contrast and brightness were adjusted in Photoshop (Adobe). Colocalization was assessed with line scans using MetaMorph (MDS Analytical Technologies). For analysis of mitochondrial membrane potential in cells, mitochondrial voxels in each image (the cytochrome c channel threshold was ≧400 au) were segregated into Parkin-positive (the YFP-Parkin channel threshold was ≧1,100 au) or Parkin-negative subsets, and MitoTracker intensity for each voxel was measured using Volocity software (Improvision). For each cell, the mean MitoTracker intensity per voxel was calculated for the Parkin-positive and Parkin-negative subsets. The difference in mean Mito-Tracker intensity between the Parkin-positive and Parkin-negative subsets was calculated using a paired t test. Western blotting HeLa cells stably expressing YFP-Parkin, HEK293 cells, and rat cortical neurons 2 days in vitro were harvested and fractionated as described previously (Karbowski et al., J. Cell Biol. 178: 71-84, 2007). Samples were run on SDS-PAGE and immunoblotted with the following antibodies: polyclonal rabbit anti-GFP (Invitrogen), mouse monoclonal anti-Parkin (PRK8), and mouse monoclonal anti-Porin 31HL (EMD).

### Electron Microscopy

HeLa cells transfected with YFP-Parkin for 18 hours were sorted for YFP using FACS. After sorting, 99.7% of cells contained a detectable YFP signal. After overnight culture, cells were treated with 10 μM CCCP for 48 h, fixed with 4% glutaraldehyde in 0.1 N sodium-cacodylate at room temperature for 1 hour, and processed for electron microscopy using a standard protocol. 22 cells expressing Parkin and 22 untransfected cells were randomly selected and imaged at 8,000× magnification by transmission electron microscope (200CX; JEOL Ltd.) and a digital camera system (XR-100; Advanced Microscopy Techniques, Corp.). The area of cytoplasm in each cell was calculated using National Institutes of Health ImageJ.

The experiments described in Example 2 were carried out using the following methods and materials.

### Cell Culture.

HeLa YFP-Parkin, PINK1+/+ SV40 transformed MEFs cells, PINK1−/− SV40 transformed MEF, M17 neuroblastoma control shRNA, M17 neuroblastoma PINK1, and Mfn1/2−/− MEF cell lines have been described previously (Narendra (2008) J Cell Biol 183: 795-803). PINK1+/+ and PINK1−/− primary MEFs were isolated from embryos using a standard protocol (Gautier (2008) Proc Natl Acad Sci USA 105: 11364-11369). Parkin+/+ and Parkin−/− transformed MEFs were created by isolation of primary cells from embryos of B6.129S4-Park2tm1Shn/J mice (Jackson Labs), using a standard protocol (Gautier (2008) Proc Natl Acad Sci USA 105: 11364-11369), followed by retroviral transduction of SV40 (Applied Biological Materials, Inc.). YFP-Parkin, YFPParkin mutants, mCherry-Parkin, PINK1-YFP, PINK1 KD-YFP, PINK1 Δ1-110-YFP, and Opa3-PINK1 Δ1-110-YFP are in C1 or N1 Clontech vectors. PINK1WT-V5, PINK1 KD-V5, and PINK1 Δ1-156-V5 are in pDest40 vector (Invitrogen). PINK1 patient mutations are in pLenti-V5 vector (Invitrogen). PINK1-myc is in a pCMBTNT vector (Promega).

### Confocal Microscopy.

Confocal microscopy of fixed samples, scoring of Parkin recruitment and Parkininduced mitophagy, and live cell imaging were performed as described previously (Narendra (2008) J Cell Biol 183: 795-803). Experiments in Mfn1/2 null cells were performed as described previously with minor modifications.

### Western Blot.

For PINK1 experiments, cells were fractionated using the Mitochondria Isolation Kit (Pierce), according to manufacturer's specifications with slight modifications. To isolate integral membrane proteins, membrane fractions obtained as above were carbonate extracted with 0.1M Na2CO3 fresh cold buffer and membranes were pelleted, as described in the supplemental methods. For Parkin experiments, cells were fractionated as described previously, with minor modifications described in the supplemental methods (Narendra (2008) J Cell Biol 183: 795-803). The following primary antibodies were used: anti-Parkin (PRK8) monoclonal (Santa Cruz), anti-Tom20 polyclonal (Santa Cruz), anti-cytochrome c monoclonal (BD Biosciences), anti-PINK1 polyclonal (Novus Biologicals), anti-VDAC monoclonal (Calbiochem), anti-GAPDH polyclonal (Sigma-Aldrich), anti-Tubulin monoclonal (Sigma-Aldrich), anti-V5 monoclonal (Invitrogen), anti-GFP polyclonal (Invitrogen).

### Cell Culture.

HeLa YFP-Parkin, E18 Rat cortical neurons, PINK1^{+/+} SV40 transformed MEFs cells, PINK1^{-/-} SV40 transformed MEF, M17 neuroblastoma control shRNA, M17 neuroblastoma PINK1, Mfn1/2^{-/-} MEF, and Parl^{-/-} MEF cell lines have been described previously (Narendra (2008) J Cell Biol 183: 795-803). PINK1^{+/+} and PINK1^{-/-} primary MEFs were isolated from embryos using a standard protocol (Gautier (2008) Proc Natl Acad Sci USA 105: 11364-11369). Parkin^{+/+} and Parkin^{-/-} transformed MEFs were created by isolation of primary cells from embryos of B6.129S4-Park2^{tm1Shn}/J mice (Jackson Labs), using a standard protocol (Gautier (2008) Proc Natl Acad Sci USA 105: 11364-11369), followed by retroviral transduction of SV40 (Applied Biological Materials, Inc.). YFP-Parkin, YFP-Parkin mutants, mCherry-Parkin, PINK1-YFP, PINK1 KD-YFP, PINK1 Δ1-110-YFP, and Opa3-PINK1 Δ1-110-YFP are in C1 or N1 Clontech vectors. PINK1WT-V5, PINK1 KD-V5, and PINK1 Δ1-156-V5 are in pDest40 vector (Invitrogen). PINK1 patient mutations are in the pLenti-V5 vector (Invitrogen). PINK1-myc is in a pCMBTNT vector (Promega). The PARL shRNA construct targeting (5′ CCAACTTGGAGCTTCTAGTAAGTTCTCTACTAGAAGCTCCAAGTTGG 3′) is in the pSuper-GFP vector. To make FRB-PINK1 (111-581)-YFP and Tom20 (1-33)-FKBP, PCR fragments containing PINK1 (111-581)-YFP and Tom20 (1-33) were cloned into the BamHI site of the pC₄-R_{H}E vector and the EcoRI and XbaI sites of pC₄M-F2E vectors, respectively (ARIAD Pharmaceuticals). The rapamycin analogue AP21967 was obtained from ARIAD Pharmaceuticals. The PARL shRNA construct targeting (5′ CCAACTTGGAGCTTCTAGTAAGTTCTCTACTAGAAGCTCCAAGTTGG 3′) is in the pSuper-GFP vector (OligoEngine).

### Confocal Microscopy.

Confocal microscopy of fixed samples, scoring of Parkin recruitment and Parkin-induced mitophagy, and live cell imageing were performed as described previously (Narendra (2008) J Cell Biol 183: 795-803). Experiments in Mfn1/2 null cells were performed as described previously with minor modifications described in the supplemental methods (Narendra (2008) J Cell Biol 183: 795-803).

### Immunoblotting and Immunocytochemistry.

For PINK1 experiments, cells were fractionated using the Mitochondria Isolation Kit (Pierce), according to manufacturer's specifications with slight modifications described in the supplemental methods. To isolate integral membrane proteins, membrane fractions obtained as above were carbonate extracted with 0.1M Na₂CO₃ fresh cold buffer and membranes were pelleted, as described in the supplemental methods. For Parkin experiments, cells were fractionated as described previously, with minor modifications detailed in the supplemental methods (Narendra (2008) J Cell Biol 183: 795-803). The protease protection assay was performed as described previously (Chen (2005) J Biol Chem 280: 26185-26192). Cells were fixed and immunostained as described previously (Narendra (2008) J Cell Biol 183: 795-803). The following primary antibodies were used: anti-Parkin (PRK8) monoclonal (Santa Cruz), anti-Tom20 polyclonal (Santa Cruz), anti-cytochrome c monoclonal (BD Biosciences), anti-PINK1 polyclonal (Novus Biologicals), anti-VDAC monoclonal (Calbiochem), anti-GAPDH polyclonal (Sigma-Aldrich), anti-Tubulin monoclonal (Sigma-Aldrich), anti-V5 monoclonal (Invitrogen), anti-GFP polyclonal (Invitrogen), anti-TIM23 monoclonal (BD Biosciences), and anti-Hsp60 monoclonal (Stressgen).

### Quantitative RT-PCR.

qRT-PCR of PINK1 mRNA levels was performed as described in detail previously (Hurley (2006) Biochem J 399: 361-372).

Materials and methods used in Example 3 are provided below.

### Cells and Culture

The Cytb 3.0 cybrid cell line was a gift from Dr. Carlos Moraes and the COXICA65 cell line has been described previously (Bruno et al. Am J Hum Genet. 65, 611-20 (1999). Cybrid cells were cultured in the DMEM containing high glucose (4.5 g/L), 2 mM sodium pyruvate, 1 mM L-glutamate and 50 μg/L of uridine (Sigma). 143B cell (ATCC) was cultured in the medium same as cybrid plus 50 μg/L of 5-bromo-2′-deoxyuridine (Sigma). HeLa cells used for expressing Twinkle (a gift from Dr. Hans Spelbrink) were cultured in DMEM medium containing high glucose (4.5 g/L), 1 mM sodium pyruvate, 2 mM L-glutamate, 10 mM HEPES and 1× non-essential amino acids. All cell culture supplies were obtained from GIBCO unless otherwise indicated.

### Fluorescent Activated Cell Sorting (FACS)

Cells in 10-cm diameter plate were transfected with 2 μg of plasmid DNA by Effectene (Invitrogen) following the manufacturer's instruction. Two days following transfection the cells were split into two 10-cm diameter plates and cultured for 3 days in culture medium containing 400 μg/ml G418 (Sigma). After G418 selection, cell were trypsinized, washed with medium once and resuspended in sorting medium (1×HBSS and 10% FBS). After sorting cells were cultured in medium containing 1× triple antibiotic (GIBCO) until next split. For membrane potential measurements, 4×10⁵ cells/well were seeded in a 6-well plate and cultured for 2 days. The cells were stained with 100 nM TMRE (Molecular Probe) in PBS (7.4) for 30 minutes at 37° C., trypsinized, and resuspended in sorting medium for FACS analysis.

### Fluorescence Microscopy

Cells for fluorescence microscopy were transfected with Fugene 6 (Roche). Cells were fixed with 4% paraformaldehyde (EMS) in PBS, treated with 0.15% of TritonX-100 and blocked with 10% BSA. Cells were incubated with rabbit anti-Tom20 (1:1000) (Santa Cruz), mouse anti-FLAG (Invitrogen) or mouse anti-COXI (Invitrogen) for 2 hs, washed with 10% BSA for 3 times, incubated with AlexaFluor Goat anti-rabbit or anti-mouse IgG (1:500)(Invitrogen) for 1 h and washed with PBS for 10 minutes 2 times. The samples were stored in PBS and imaged on a Zeiss LSM510 microscope (63×/1.4 Oil DIC Plan Apo objective, 40×/1.3 Oil Plan Neo-Fluar objective). For membrane potential, cells were incubated with 2.5 nM of TMRE (Invitrogen) culture medium for 1 h and imaged.

### PCR-RFLP

The PCR-RFLP method basically is follow the methods described in the previous paper (Bruno, et al. Am J Hum Genet 65, 611-20 (1999)). Total DNA was extracted from cells by DNeasy blood and tissue kit (QIAGEN). 450 ng of DNA was used as a template in PCR. The forward primer, 5′-ggcttcctagggtttatcgtgtgagcac-3′, and the reversed primer, 5′-ggccacctacggtgaaaagaaagatgaagc-3′, were used for COXICA65 cybrid. PCR amplification was performed as follows: the first step at 94° C. for 2 min; 30 cycles each of 94° C. for 20 sec, 58° C. for 30 seconds and 72° C. for 30 seconds; and final step at 72° C. for 7 mM PCR product was purified using a gel extraction kit (QIAGEN). 300 ng purified PCR product was digested with AluI (NEB) in 50 μl. 10 μl (60 ng) of digested product was loaded and separated in a 10% TBE polyacrylamide gel (Invitrogen). After electrophoresis, the gel was stained with 2 μg/ml ethidium bromide for 10 mM, destained with water for 5 minutes and imaged. For ³²P-labeling, 1 μl (20 μCi) of [α-³²P]-dCTP (Perkin Elmer) was added in each sample at the last cycle of PCR reaction. The procedures after PCR were as same as the described above. After electrophoresis, the radioactive signals were detected by phosphoimage system and scanned by STORM 860 (GE Healthcare). The intensity was quantified by the ImageQuant 5.1 program (GE Healthcare).

### Cytochrome c Oxidase Activity Assay

Cells grown on 10 cm diameter dishes were collected in extraction buffer [10 mM HEPE, 10 mM NaCl₂, 1.5 mM MgCl₂,4 mM NaF, 100 μM NaOVac, 1× protease cocktail (Roche)] and lysed by 20 passages through a 25 gauge needle. Samples were spun at 500×g for 5 minutes to pellet nuclei. The supernatant was then spun at 8000 rpm for 10 minutes to pellet the mitochondrial rich heavy membrane fraction. The pellet was washed with extraction buffer once and resuspended in 1× enzyme dilution buffer containing 1 mM n-dodecyl-p-maltoside (CYTOCX1 kit, Sigma). The COXI activity assay was performed according to the manufacturer's instructions. Fresh 10 μg of mitochondrial protein and 10 μM Ferrocytochrome c substrate were used in each assay. SpectraMax plus³⁸⁴ (Molecular Devices) was used to measure A₅₅₀ every 10 seconds for 1 mM The Vmax of each sample was calculated by SOFTmax PRO (Molecular Devices). The mean and standard deviation were calculated from three experiments

The experiments reported above in Examples 4 and 5 were carried out with the following methods and materials.

### Cell Culture and Chemicals

HeLa cells and MEFs were maintained in DMEM supplemented with 10% fetal calf serum, 20 mM L-glutamine, 1 mM sodium pyruvate, 1×MEM non-essential amino acids, and penicillin/streptomycin. HeLa cells stably expressing YFP-Parkin were grown under selection in 300 μg/ml hygromycin (Sigma-Aldrich). All chemicals for cell culture were purchased from Invitrogen. PARL KO and WT MEFs were gifted by L. Pellegrini (University of Cambridge, Cambridge, UK), and PINK1 KO MEFs by Z. Zhang (Burnham Institute for Medical Research, La Jolla, Calif.).

### Constructs and Mutagenesis

PARL siRNA (5′-AAATCCAGGGTCCAGAGTTAT-C′) were synthesized by Qiagen. In order to achieve efficient knockdown of PARL, cells were transfected twice over a period of 96 hrs. PINK1-V5/His was a gift from M. Cookson (National Institute of Health, Bethesda, Md.). For site-directed mutagenesis, primers were designed using Primer X, web-based mutagenic primer design program (http://www.bioinformatics.org/primerx/), and produced by Operon. 15 rounds of PCR reactions were performed using Phusion DNA polymerase (Finnzymes) and WT PINK1-YFP constructs as a template. Introduction of point mutations were confirmed by sequencing.

### Transfection

Cells were cultured in borosilicated chamber slides for imaging, 6-well plates for whole cell lysates, and 100 or 150 mm culture dish for subcellular fractionation. One day after seeding, cells were transfected with indicated constructs using Fugene HD (Roche) or Lipofectamine 2000 (Invitrogen) according to the manufacturer's guidelines.

### Western Blotting

For whole cell lysates, cells were washed twice with cold-PBS and then directly lysed with 1× sample buffer. For mitochondrial fraction, cells were fractionated as described previously (Narendra et al., 2008) and below, and then mitochondrial pellets were lysed with 1× sample buffer. 20 μg of proteins were separated on 4-12% Tris-glycine or Bis-Tris SDS-PAGE. The following antibodies were used: anti-PINK1 (Novus Biologicals), anti-PARL N-terminal (a gift from L. Pellegrini), anti-Tom20 (Santa Cruz Biotechnology, Inc.), anti-AIF (Sigma-Aldrich) and antiHtra2/Omi (R&D systems) polyclonal antibodies; anti-VDAC (Calbiochem), anti-Tubulin, anti-βactin (Sigma-Aldrich), anti-Hsp70 (Cell Signaling Technologies), anti-Tom20 (BD), anti-Tim23 and anti-cytochrome C (BD) monoclonal antibodies.

### Subcellular Fractionation, Proteinase K Treatment and Alkaline Extraction of Isolated Mitochondria

Prior to fractionation, HeLa cells were treated with 50 μM MG132 for 10 h followed by 10 μM CCCP for 3 hours in order to accumulate both FL and 52 kDa forms of PINK1. Harvested cells were homogenized using a teflon pestle (Thomas Scientific) in 20 mM Hepes-KOH (pH 7.6), 220 mM mannitol, 70 mM sucrose, 1 mM EDTA, 0.5 mM phenylmethylsulfonyl fluoride (PMSF), 2 mg/ml BSA and centrifuged at 800 g at 4 C for 10 min to obtain a postnuclear supernatant. Mitochondria were pelleted by centrifugation at 10 000 g at 4 C for 20 mM The supernatant fraction was centrifuged further for 30 mM at 100 000 g to obtain a cytosolic protein fraction. Cytosolic fractions were concentrated using trichloroacetic acid precipitation. Mitochondrial samples treated by alkaline extraction were resuspended in freshly prepared 0.1 M Na2CO3, pH 11.5 and incubated on ice for 30 min with occasional vortexing. Membranes were isolated by centrifugation at 100 000 g for 30 min at 4 C and solubilized in SDS-PAGE loading dye.

For PK protection assays, mitochondria freshly isolated from HeLa cells as described above were resuspended in 20 mM Hepes-KOH pH 7.4, 250 mM sucrose, 80 mM KOAc, 5 mM MgOAc and incubated with various concentrations of PK (Sigma) for 30 mM on ice. Digestion was stopped with 1 mM PMSF followed by trichloroacetic acid precipitation of samples, separation by SDSPAGE and western blotting.

In Vitro Import of Pink1 into Isolated Mitochondria

Generation of radiolabeled PINK1 precursor was performed by in vitro transcription, followed by translation using rabbit reticulocyte lysates (Promega) in the presence of ³⁵S-methionine/cysteine protein labeling mix (Perkin Elmer) as previously described (Lazarou et al., 2008, Mol Cell Biol. 27:4228-37). PINK1 translation products were incubated with freshly isolated mitochondria in import buffer (20 mM Hepes-KOH pH 7.4, 250 mM sucrose, 80 mM KOAc, 5 mM MgOAc, 5 mM methionine, 1 mM DTT, 5 mM ATP) at 24 C for various times as indicated in the figure legend. Dissipation of membrane potential was performed using 1 μM CCCP. Samples subjected to protease treatment were incubated on ice for 10 min in 5 μg/ml PK before protease inactivation with 1 mM PMSF. Mitochondrial pellets (50 μg) were precipitated using TCA and subjected to SDS-PAGE. Radiolabeled PINK1 was detected by digital autoradiography.

### Immunocytochemistry and Live Cell Imaging

For immunocytochemistry, cultured cells in borosilicated chamber slides were fixed with 4% paraformaldehyde in PBS (USB) and permeabilized with the indicated detergent. After 30 min blocking with 10% BSA in PBS, cells were stained with the following primary antibodies: antiTom20 polyclonal antibody (Santa Cruz Biotechnology, Inc.) and anti-cytochrome C monoclonal antibody (BD), or anti-Tom20 monoclonal antibody (BD) and anti-GFP polyclonal antibody (Invitrogen) and then with the following secondary antibodies: goat anti-mouse/rabbit IgG antibody conjugated with Alexa Fluor 594/647, respectively. In case of live cell imaging, cells were pulsed with 600 nM TMRE for 5 min to evaluate mitochondrial membrane potential or 10 nM Mitotracker Red for 30 min to see the mitochondrial morphology. Cells were imaged using an inverted microscope (LSM510 Meta; Carl Zeiss) with 63×/1.4 oil DIC Plan Apo objective. Image contrast and brightness were adjusted in LSM image browser (Carl Zeiss).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of reducing the number of defective mitochondria in a cell, the method comprising
(a) identifying a cell as having an increased number of defective mitochondria;
(b) contacting the cell with an agent that increases Pink1 or Parkin expression or biological activity in the cell, thereby reducing the number of defective mitochondria in the cell.

2. (canceled)

3. The method of claim 1, wherein the cell is an ocular cell, neuron, muscle cell, or oocyte.

4-5. (canceled)

6. The method of claim 1, wherein the agent is an expression vector encoding a Pink1 or Parkin polynucleotide.

7. (canceled)

8. The method of claim 1, wherein the defective mitochondria has a dysfunction selected from the group consisting of a reduction in the activity of a mitochondrial enzyme, reduced electron transport chain (ETC) activity, diminished membrane potential, increased reactive oxygen species production, mitochondrial fragmentation, calcium dysregulation, and a mutation in mitochondrial DNA (mtDNA).

9. (canceled)

10. The method of claim 1, wherein the agent is an uncoupling agent, carbonylcyanide-3-chlorophenylhydrazone (CCCP), or dinitrophenol.

11-13. (canceled)

14. A method of treating or preventing a mitochondrial disease in a subject, the method comprising identifying the subject as having an increased number of defective mitochondria and administering to the subject an effective amount of an agent that increases Pink1 or Parkin expression or biological activity in a cell, thereby treating the disease.

15. The method of claim 14, wherein the agent is a mammalian expression vector encoding a Parkin or PINK1 polypeptide or fragment thereof.

16. The method of claim 14, wherein the disease is associated with a mitochondrial dysfunction selected from the group consisting of a reduction in the activity of a mitochondrial enzyme, reduced electron transport chain (ETC) activity, diminished membrane potential, increased reactive oxygen species production, mitochondrial fragmentation, calcium dysregulation, and a mutation in mitochondrial DNA (mtDNA).

17. The method of claim 14, wherein the disease is cancer; diabetes mellitus; a hereditary mitochondrial disease selected from the group consisting of Neurogenic muscular weakness-Ataxia-Retinitis pigmentosa (NARP), Multiple Sclerosis-like Syndrome (MSS); Maternally Inherited CardioMyopathy (MCIM); Progressive External Ophthalmoplegia (PEO); Myoclonic Epilepsy with Ragged-Red Fibers (MERRF); Myoneurogastrointestinal disorder and encephalopathy (MNGIE), Pearson Marrow syndrome, Kearns-Sayre-CPEO, Leber hereditary optic neuropathy (LHON), Aminoglycoside-associated deafness, Diabetes with deafness, Luft disease, Leigh syndrome (Complex I, COX, PDH), Alpers Disease, MCAD, SCAD, SCHAD, VLCAD, LCHAD, Glutaric aciduria II, and Lethal infantile cardiomyopathy; or a mitochondrial disease that is not Parkinson's disease.

18. (canceled)

19. The method of claim 1 or 14, wherein the method increases autophagy of small defective mitochondria that lack membrane potential.

20. The method of claim 1 or 14, wherein the method increases biogenesis of new mitochondria.

21-35. (canceled)

36. A method for identifying a compound useful for the treatment of a mitochondrial disease, the method comprising:
(a) contacting a cell with a compound and an agent that disrupts mitochondrial function; and
(b) identifying a reduction in the number of defective mitochondria in the cell relative to a control cell not contacted with the candidate compound, wherein a compound that reduces the number of defective mitochondria in the cell is identified as useful for the treatment of a mitochondrial disease; or
a method for identifying a compound useful for the treatment of a subject having a mitochondrial disease, the method comprising:
(a) contacting a cell derived from the subject with a compound and an agent that disrupts mitochondrial function; and
(b) identifying a reduction in the number of defective mitochondria in the cell relative to a control cell not contacted with the candidate compound, wherein a compound that reduces the number of defective mitochondria in the cell is identified as useful for the treatment of the subject having mitochondrial disease; or
a method for identifying a compound useful for the treatment of a mitochondrial disease, the method comprising:
(a) contacting a cell with a compound and an agent that disrupts mitochondrial function; and
(b) identifying an increase of PINK1 or Parkin associated with mitochondria in the cell relative to a control cell not contacted with the candidate compound, wherein a compound that increases PINK1 or Parkin association with mitochondria in the cell is identified as useful for the treatment of a mitochondrial disease; or
a method for identifying a compound useful for the treatment of a mitochondrial disease, the method comprising:
(a) contacting a cell comprising a mutation in mitochondrial DNA with a compound; and
(b) identifying a reduction in the number of defective mitochondria in the cell relative to a control cell not contacted with the candidate compound, wherein a compound that reduces the number of defective mitochondria in the cell is identified as useful for the treatment of a mitochondrial disease; or
a method for identifying a compound useful for the treatment of a mitochondrial disease, the method comprising:
(a) contacting a cell with a compound; and
(b) identifying an increase of PINK1 or Parkin associated with mitochondria in the cell relative to a control cell not contacted with the candidate compound, wherein a compound that increases PINK1 or Parkin association with mitochondria in the cell is identified as useful for the treatment of a mitochondrial disease; or
a method for identifying a compound useful for the treatment of a subject having a mitochondrial disease, the method comprising:
(a) contacting a cell derived from the subject with a compound; and
(b) identifying a reduction in the number of defective mitochondria in the cell relative to a control cell not contacted with the candidate compound, wherein a compound that reduces the number of defective mitochondria in the cell is identified as useful for the treatment of said mitochondrial disease in the subject; or
a method for identifying a compound useful for the treatment of Parkinson's disease, the method comprising:
(a) contacting a dopaminergic cell with a candidate compound and an agent that disrupts mitochondrial function; and
(b) identifying a reduction in the number of defective mitochondria in the cell relative to a control cell not contacted with the candidate compound, wherein a compound that reduces the number of defective mitochondria in the cell is identified as useful for the treatment of a Parkinson's disease; or
a method for identifying a compound useful for the treatment of Parkinson's disease, the method comprising:
(a) contacting a cell comprising a mutation in Pink1 or Parkin with a candidate compound; and
(b) identifying a reduction in the number of defective mitochondria in the cell relative to a control cell not contacted with the candidate compound, wherein a compound that reduces the number of defective mitochondria in the cell is identified as useful for the treatment of a Parkinson's disease.

37-45. (canceled)

46. The method of claim 36, wherein the compound is a polypeptide, polynucleotide, small chemical compound, or microRNA.

47-50. (canceled)

51. A method for ameliorating Parkinson's disease in a subject, the method comprising administering to the subject an agent that reduces the biological activity or expression of PARL.

52. The method of claim 51, wherein the agent is i) an inhibitory nucleic acid molecule that reduces the expression of PARL polynucleotide or polypeptide; or ii) a protease inhibitor that reduces PARL proteolytic activity.

53. The method of claim 52, wherein the inhibitory nucleic acid molecule is an siRNA, shRNA, or antisense polynucleotide.

54. (canceled)

55. A pharmaceutical composition formulated for use in the method of claim 14, the composition comprising an effective amount of Parkin or Pink1 in a pharmaceutically acceptable excipient.

56. A kit for treating a mitochondrial disease comprising the pharmaceutical composition of claim 55, wherein the kit further comprises instructions for identifying a subject in need of such treatment and directions for administering the pharmaceutical composition to a subject.
